SEQUENCE LISTING (42 sequences)

SEQ ID NO: 1 - DNA, 1710 bp, Artificial Sequence
  DNA encoding Fc-SED1.320 bait expression cassette (ScSED1.320 Anchor)
atgagatttccttcaatttttactgctgttttattcgcagcatcctccgcattagctgacaagacacatacttgtccaccatgtccagctccagaattgttgggtggtccatccgttttcttgttcccaccaaagccaaaggacactttgatgatctccagaactccagaggttacatgtgttgttgttgacgtttctcacgaggacccagaggttaagttcaactggtacgttgacggtgttgaagttcacaacgctaagactaagccaagagaagagcagtacgcttccacttacagagttgtttccgttttgactgttttgcaccaggactggttgaacggtaaagaatacaagtgtaaggtttccaacaaggctttgccagctccaatcgaaaagactatctccaaggctaagggtcaaccaagagagccacaggtttacactttgccaccatccagagaagagatgactaagaaccaggtttccttgacttgtttggttaaaggattctacccatccgacattgctgttgagtgggaatctaacggtcaaccagagaacaactacaagactactccaccagttttggattctgatggttccttcttcttgtactccaagttgactgttgacaagtccagatggcaacagggtaacgttttctcctgttccgttatgcatgaggctttgcacaaccactacactcaaaagtccttgtctttgtcccctggtggtggtggtgtcgaccaattttctaattctacatcagcatcttcaacagacgtaacttccagttcttcaatatcaacttccagtggttccgtcactatcacatcttcagaagctccagaaagtgataacggtacttctactgcagcccctacagaaacctcaactgaagccccaaccactgctattcctactaatggtacatctaccgaagcaccaacaaccgccatacctacaaacggtacttctacagaagcaccaactgatactacaaccgaagctccaactacagcattgcctacaaatggtacttctactgaagccccaactgacaccactacagaagctccaaccactggtttgcctacaaacggtacaacctcagcttttccacctactacatccttaccacctagtaataccactacaaccccaccttataacccatctactgattatactacagactacacagttgtaactgaatataccacttactgtccagaacctacaaccttcactacaaatggtaaaacatacaccgttactgaaccaaccactttaacaataaccgattgtccatgcacaatcgaaaagcctacaaccacttctacaaccgaatacacagtcgttactgaatacactacatactgtccagaacctaccactttcacaaccaatggtaaaacttacacagttaccgaaccaactacattgactattacagactgtccttgcactatagaaaagtcagaagctccagaatccagtgtacctgtcacagaatccaaaggtactactacaaaggaaactggtgttaccactaaacaaacaaccgcaaatccatctttaacagtctcaactgtagtccctgtttcttcatccgccagttctcattcagttgtaattaattccaacggtgctaatgttgtcgttccaggtgctttgggtttggcaggtgttgctatgttgtttttg

SEQ ID NO: 2 - DNA, 318 bp, Artificial Sequence
  TetO7 promoter DNA sequence
ctcgatcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactcctcagtgactatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtaccctatg

SEQ ID NO: 3 - DNA, 235 bp, Artificial Sequence
  CYC-TT DNA sequence
catgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaag

SEQ ID NO: 4 - DNA, 1615 bp, Artificial Sequence
  ScMET15 gene
atcctcatgaaaactgtgtaacataataaccgaagtgtcgaaaaggtggcaccttgtccaattgaacacgctcgatgaaaaaaataagatatatataaggttaagtaaagcgtctgttagaaaggaagtttttcctttttcttgctctcttgtcttttcatctactatttccttcgtgtaatacagggtcgtcagatacatagatacaattctattacccccatccatacaatgccatctcatttcgatactgttcaactacacgccggccaagagaaccctggtgacaatgctcacagatccagagctgtaccaatttacgccaccacttcttatgttttcgaaaactctaagcatggttcgcaattgtttggtctagaagttccaggttacgtctattcccgtttccaaaacccaaccagtaatgttttggaagaaagaattgctgctttagaaggtggtgctgctgctttggctgtttcctccggtcaagccgctcaaacccttgccatccaaggtttggcacacactggtgacaacatcgtttccacttcttacttatacggtggtacttataaccagttcaaaatctcgttcaaaagatttggtatcgaggctagatttgttgaaggtgacaatccagaagaattcgaaaaggtctttgatgaaagaaccaaggctgtttatttggaaaccattggtaatccaaagtacaatgttccggattttgaaaaaattgttgcaattgctcacaaacacggtattccagttgtcgttgacaacacatttggtgccggtggttacttctgtcagccaattaaatacggtgctgatattgtaacacattctgctaccaaatggattggtggtcatggtactactatcggtggtattattgttgactctggtaagttcccatggaaggactacccagaaaagttccctcaattctctcaacctgccgaaggatatcacggtactatctacaatgaagcctacggtaacttggcatacatcgttcatgttagaactgaactattaagagatttgggtccattgatgaacccatttgcctctttcttgctactacaaggtgttgaaacattatctttgagagctgaaagacacggtgaaaatgcattgaagttagccaaatggttagaacaatccccatacgtatcttgggtttcataccctggtttagcatctcattctcatcatgaaaatgctaagaagtatctatctaacggtttcggtggtgtcttatctttcggtgtaaaagacttaccaaatgccgacaaggaaactgacccattcaaactttctggtgctcaagttgttgacaatttaaagcttgcctctaacttggccaatgttggtgatgccaagaccttagtcattgctccatacttcactacccacaaacaattaaatgacaaagaaaagttggcatctggtgttaccaaggacttaattcgtgtctctgttggtatcgaatttattgatgacattattgcagacttccagcaatcttttgaaactgttttcgctggccaaaaaccatgagtgtgcgtaatgagttgtaaaattatgtataaacctactttctctcaca

SEQ ID NO: 5 - DNA, 1482 bp, Artificial Sequence
  ScURA3 gene
caatacagacgatgataacaaaccgaagttatctgatgtagaaaaggattaaagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttctataatgacgaaaaaaaaaaaattggaaagaaaaagcttcatggcctttataaaaaggaactatccaatacctcgccagaaccaagtaacagtattttacggggcacaaatcaagaacaataagacaggactgtaaagatggacgcattgaactccaa

SEQ ID NO: 6 - DNA, 1404 bp, Artificial Sequence
  DNA encoding Anti-HER2 IgG1-G1m(f), N297A expression cassette
atgagattcccatccatcttcactgctgttttgttcgctgcttcttctgctttggctgaggttcagttggttgaatctggaggaggattggttcaacctggtggttctttgagattgtcctgtgctgcttccggtttcaacatcaaggacacttacatccactgggttagacaagctccaggaaagggattggagtgggttgctagaatctacccaactaacggttacacaagatacgctgactccgttaagggaagattcactatctctgctgacacttccaagaacactgcttacttgcagatgaactccttgagagctgaggatactgctgtttactactgttccagatggggtggtgatggtttctacgctatggactactggggtcaaggaactttggttactgtctcgagtgcttcaactaagggaccatctgtcttcccattggctccatcttcaaagtctacttcaggtggtactgctgctttgggttgcttggtcaaagactacttcccagagccagtcacagtttcttggaactctggtgctttgacttctggtgtccacactttcccagcagtcttacaatcttctggtttgtattcattgtcttctgttgttacagttccatcttcttctttgggtactcaaacttatatttgtaatgttaaccataaaccatctaatactaaagttgataagaaagttgaaccaaaatcttgtgataagactcacacttgtccaccttgcccagctccagaattgttaggtggtccttctgtcttcttgttcccaccaaagccaaaagatacattgatgatttctagaactccagaggttacatgtgtcgtcgtcgatgtctctcacgaggatcctgaggtcaagttcaactggtacgtcgacggtgttgaggtccacaacgctaagactaagccaagagaagaacaatacgcttctacatatagagttgtctctgttttgactgttttgcatcaggattggttaaatggtaaagaatataagtgtaaagtttctaataaggctttaccagcaccaattgaaaaaactatttctaaggctaagggtcaaccaagggaaccacaggtctacacattgccaccatcaagagaagaaatgactaaaaatcaagtttctttaacttgcttggttaagggtttctatccatcagatattgctgtcgagtgggaatctaatggtcagccagaaaataattataaaacaactccaccagttttggattctgatggttctttctttttatattctaaattgacagtcgataagtctaggtggcagcaaggtaacgttttctcatgctctgtcatgcacgaggctttgcacaaccactacacacaaaaatctttgtcattatctccaggt

SEQ ID NO: 7 - DNA, 456 bp, Artificial Sequence
  Gal1 promoter DNA sequence
tgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaac

SEQ ID NO: 8 - DNA, 1103 bp, Artificial Sequence
  ScLEU2 gene
atgtctgcccctatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgtcccacttccagatgaggcgctggaagcctccaagaaggttgatgccgttttgttaggtgctgtgggtggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaatttcctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggtcaaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgc

SEQ ID NO: 9 - DNA, 699 bp, Artificial Sequence
  DNA encoding MF-Pre-anti-HER2-Lc Kappa CL expression cassette
atgagattcccatccatcttcaccgccgttttgttcgctgcttcttccgctttggctgatattcaaatgactcaatctccatcttctttgtctgcttctgtcggtgatcgtgtcactattacttgtcgtgcttctcaagatgtcaacactgctgtcgcttggtatcaacagaagcccggtaaggctccaaagttgttgatttattctgcttcttttttgtactctggtgtcccatctaggttctctggttctaggtctggtactgattttactttgactatttcttctttgcaaccagaagattttgctacttattattgtcaacaacattatactacaccaccaacttttggtcaaggtaccaaggttgagatcaagagaactgttgctgctccatccgttttcattttcccaccatccgacgaacagttgaagtctggtacagcttccgttgtttgtttgttgaacaacttctacccaagagaggctaaggttcagtggaaggttgacaacgctttgcaatccggtaactcccaagaatccgttactgagcaagactctaaggactccacttactccttgtcctccactttgactttgtccaaggctgattacgagaagcacaaggtttacgcttgtgaggttacacatcagggtttgtcctccccagttactaagtccttcaacagaggagagtgt

SEQ ID NO: 10 - DNA, 672 bp, Artificial Sequence
  TRP1 gene
atgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaa

SEQ ID NO: 11 - DNA, 2175 bp, Artificial Sequence
  DNA encoding AGA1
atgactttgtctttcgcacatttcacttatttatttactattttgttgggattgactaatattgctttggcttctgatccagaaactattttggttactattactaaaactaacgatgctaacggagtcgtcacaactactgtctcaccagctttggtctctacttctactattgtccaagctggtacaactactttgtatactacttggtgtccattaactgtttctacttcatcagcagcagaaatttctccatctatttcatacgctacaacattgtcaagattttcaactttaacattgtcaactgaggtttgctcacatgaggcttgcccttcttcatctactttacctactacaacattgtcagttacttcaaagttcacttcttacatttgcccaacatgtcatactacagctatttcttcattgtctgaagtcggtactacaactgttgtttcatcttctgctatagaaccatcttcagcttctattatatctccagttacatctactttgtcttcaacaacttcttctaaccctacaactacttctttgtcatcaacatctacttctccatcttcaacttctacttctccatcatctacatctacttcatcttcttctacttctacttcatcttcttctacttcaacttcatcttcttctacttctacttctccatcatctacatcaacatcatcatctttgacatctacatcttcttcttcaacatctacttcacagtcttctacttctacatcttcttcatcaacttcaacatctccttcttcaacatctacatcttcttcttcaacatcaacatcaccatcatctaaatcaacttcagcttcatctacttctacatcttcttattctacttctacttctccatcattgacttcttcttcaccaacattggcttctacatcaccatcttctacttctatatcttctacatttactgattcaacttcttctttgggttcatcaatagcttcttcttcaacatctgtttctttgtactctccatctactcccgtttactcagtcccttctacatcatctaacgttgctactccttctatgacatcttctactgtcgagactactgtttcatctcaatcatcttctgaatatattactaaatcttcaatttctactactattccatctttttctatgtcaacatatttcactacagtttctggtgttacaactatgtacactacttggtgcccatactcttcagaatctgaaacttcaacattgacttctatgcatgaaacagtcacaactgacgctactgtttgcacacacgagtcttgcatgccatctcagactacttcattaattacttcttctattaaaatgtctactaaaaatgttgctacttctgtttcaacatctacagttgaatcttcttacgcttgttctacttgcgctgaaacttcacattcatattcttcagttcaaactgcttcttcttcttctgttactcagcaaacaacttcaacaaaatcatgggtctcatctatgacaacatctgatgaagactttaacaaacacgctactggtaaatatcatgttacttcttctggaacttctacaatttctacatctgtttctgaagctacatctacttcttctatagattctgagtctcaagaacaatcttctcacttattatcaacatctgtcttgtcttcttcttctttatctgctacattgtcatctgactcaactattttgttattctcttcagtctcttctttgtcagttgaacaatctccagtcactactttgcaaatatcatcaacttctgagattttacaaccaacatcttctacagctatagctactatttctgcttctacttcttctttatctgctacttctatatctacaccatctacatctgttgaatctactattgagtcttcttctttgacacctactgtttcttctattttcttatcttcttcttctgctccatcttctttgcaaacatctgttacaactactgaagtttctactacatctatttcaattcaatatcagacttcttctatggtcacaatttctcaatacatgggttctggttctcaaactagattgccattgggtaaattggtttttgctattatggcagtcgcttgtaatgttattttctct

SEQ ID NO: 12 - DNA, 993 bp, Artificial Sequence
  DNA encoding Fc-AGA2 bait expression cassette
atgagatttccttcaatttttactgctgttttattcgcagcatcctccgcattagctgacaagacacatacttgtccaccatgtccagctccagaattgttgggtggtccatccgttttcttgttcccaccaaagccaaaggacactttgatgatctccagaactccagaggttacatgtgttgttgttgacgtttctcacgaggacccagaggttaagttcaactggtacgttgacggtgttgaagttcacaacgctaagactaagccaagagaagagcagtacgcttccacttacagagttgtttccgttttgactgttttgcaccaggactggttgaacggtaaagaatacaagtgtaaggtttccaacaaggctttgccagctccaatcgaaaagactatctccaaggctaagggtcaaccaagagagccacaggtttacactttgccaccatccagagaagagatgactaagaaccaggtttccttgacttgtttggttaaaggattctacccatccgacattgctgttgagtgggaatctaacggtcaaccagagaacaactacaagactactccaccagttttggattctgatggttccttcttcttgtactccaagttgactgttgacaagtccagatggcaacagggtaacgttttctcctgttccgttatgcatgaggctttgcacaaccactacactcaaaagtccttgtctttgtcccctaaagctagcggtggtggtggttctgaagctgcagcaaaagaggcagctgctaagcaagagttgactactatttgtgaacaaattccatctccaactttggaatctactccatattctttgtctactactactattttggctaatggtaaagctatgcaaggtgtttttgaatattataaatctgttactttcgtctctaactgcggttctcacccatctactacttctaagggttctccaattaatactcaatatgttttt

SEQ ID NO: 13 - protein, 338 aa, Artificial Sequence
  ScSED1.320 (1-18 SED1 signal sequence)
MKLSTVLLSAGLASTTLAQFSNSTSASSTDVTSSSSISTSSGSVTITSSEAPESDNGTSTAAPTETSTEAPTTAIPTNGTSTEAPTTAIPTNGTSTEAPTDTTTEAPTTALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 14 - protein, 419 aa, Artificial Sequence
  ScSED1.401 (1-18 SED1 signal sequence)
MKLSTVLLSAGLASTTLAQFSNSTSASSTDVTSSSSISTSSGSVTITSSEAPESDNGTSTAAPTETSTEAPTTAIPTNGTSTEAPTDTTTEAPTTALPTNGTSTEAPTDTTTEAPTTAIPTNGTSTEAPTDTTTEAPTTALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 15 - protein, 448 aa, Artificial Sequence
  ScSED1.430 (1-18 SED1 signal sequence)
MKLSTVLLSAGLASTTLAQFSNSTSASSTDVTSSSSISTSSGSVTITSSEAPESDNGTSTAAPTETSTEAPTTAIPTNGTSTEAPTDTTSEAPTTALPTNGTSTEAPTDTTTEAPTTALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 16 - protein, 499 aa, Artificial Sequence
  ScSED1.481 (1-18 SED1 signal sequence)
MKLSTVLLSAGLASTTLAQFSNSTSASSTDATSSSSISTSSGSVTITSSEAPESDNGTSTAAPTETSTEAPTTAIPTNGTSTEAPTDTTTEAPTTALPTNGTSTEAPTDTTTEAPTTALPTNGTSTEAPTDTTTEAPTTALPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 17 - protein, 320 aa, Artificial Sequence
  ScSED1.320 (without SED1 signal sequence)
QFSNSTSASSTDVTSSSSISTSSGSVTITSSEAPESDNGTSTAAPTETSTEAPTTAIPTNGTSTEAPTTAIPTNGTSTEAPTDTTTEAPTTALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 18 - protein, 401 aa, Artificial Sequence
  ScSED1.401 (without SED1 signal sequence)
QFSNSTSASSTDVTSSSSISTSSGSVTITSSEAPESDNGTSTAAPTETSTEAPTTAIPTNGTSTEAPTDTTTEAPTTALPTNGTSTEAPTDTTTEAPTTAIPTNGTSTEAPTDTTTEAPTTALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 19 - protein, 430 aa, Artificial Sequence
  ScSED1.430 (without SED1 signal sequence)
QFSNSTSASSTDVTSSSSISTSSGSVTITSSEAPESDNGTSTAAPTETSTEAPTTAIPTNGTSTEAPTDTTSEAPTTALPTNGTSTEAPTDTTTEAPTTALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 20 - protein, 481 aa, Artificial Sequence
  ScSED1.481 (without SED1 signal sequence)
QFSNSTSASSTDATSSSSISTSSGSVTITSSEAPESDNGTSTAAPTETSTEAPTTAIPTNGTSTEAPTDTTTEAPTTALPTNGTSTEAPTDTTTEAPTTALPTNGTSTEAPTDTTTEAPTTALPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 21 - protein, 735 aa, Artificial Sequence
  DNA encoding human IgG1 Fc N297A mutein with S. cerevisiae pre-pro alpha-mating factor signal sequence at the 5′ end
ATGAGATTTCCTTCAATTTTTACTGCTGTTTTATTCGCAGCATCCTCCGCATTAGCTGACAAGACACATACTTGTCCACCATGTCCAGCTCCAGAATTGTTGGGTGGTCCATCCGTTTTCTTGTTCCCACCAAAGCCAAAGGACACTTTGATGATCTCCAGAACTCCAGAGGTTACATGTGTTGTTGTTGACGTTTCTCACGAGGACCCAGAGGTTAAGTTCAACTGGTACGTTGACGGTGTTGAAGTTCACAACGCTAAGACTAAGCCAAGAGAAGAGCAGTACGCTTCCACTTACAGAGTTGTTTCCGTTTTGACTGTTTTGCACCAGGACTGGTTGAACGGTAAAGAATACAAGTGTAAGGTTTCCAACAAGGCTTTGCCAGCTCCAATCGAAAAGACTATCTCCAAGGCTAAGGGTCAACCAAGAGAGCCACAGGTTTACACTTTGCCACCATCCAGAGAAGAGATGACTAAGAACCAGGTTTCCTTGACTTGTTTGGTTAAAGGATTCTACCCATCCGACATTGCTGTTGAGTGGGAATCTAACGGTCAACCAGAGAACAACTACAAGACTACTCCACCAGTTTTGGATTCTGATGGTTCCTTCTTCTTGTACTCCAAGTTGACTGTTGACAAGTCCAGATGGCAACAGGGTAACGTTTTCTCCTGTTCCGTTATGCATGAGGCTTTGCACAACCACTACACTCAAAAGTCCTTGTCTTTGTCCCCTGGT

SEQ ID NO: 22 - protein, 245 aa, Artificial Sequence
  human IgG1 Fc N297A mutein (C-terminal K-) with 1-19 S. cerevisiae pre-pro alpha-mating factor signal sequence at the 5′ end
MRFPSIFTAVLFAASSALADKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 23 - protein, 5 aa, Artificial Sequence
  Linker
GGGVD

SEQ ID NO: 24 - protein, 250 aa, Artificial Sequence
  human IgG1 Fc N297A mutein (C-terminal K-) with GGGVD linker and 1-19 S. cerevisiae pre-pro alpha- mating factor signal sequence
MRFPSIFTAVLFAASSALADKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGVD

SEQ ID NO: 25 - protein, 19 aa, Artificial Sequence
  S. cerevisiae pre-pro alpha- mating factor signal sequence
MRFPSIFTAVLFAASSALA

SEQ ID NO: 26 - protein, 226 aa, Artificial Sequence
  human IgG1 Fc N297A mutein (C-terminal K-) without signal sequence
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 27 - protein, 551 aa, Artificial Sequence
  ScSED1.320 IgG1 Fc N297A mutein (without signal sequence) (ScSED1.320 Anchor)
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGVDQFSNSTSASSTDVTSSSSISTSSGSVTITSSEAPESDNGTSTAAPTETSTEAPTTAIPTNGTSTEAPTTAIPTNGTSTEAPTDTTTEAPTTALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 28 - protein, 632 aa, Artificial Sequence
  ScSED1.401 IgG1 Fc N297A mutein (without signal sequence) (ScSED1.401 Anchor)
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGVDQFSNSTSASSTDVTSSSSISTSSGSVTITSSEAPESDNGTSTAAPTETSTEAPTTAIPTNGTSTEAPTDTTTEAPTTALPTNGTSTEAPTDTTTEAPTTAIPTNGTSTEAPTDTTTEAPTTALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 29 - protein, 661 aa, Artificial Sequence
  ScSED1.430 IgG1 Fc N297A mutein (without signal sequence) (ScSED1.430 Anchor)
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGVDQFSNSTSASSTDVTSSSSISTSSGSVTITSSEAPESDNGTSTAAPTETSTEAPTTAIPTNGTSTEAPTDTTSEAPTTALPTNGTSTEAPTDTTTEAPTTALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 30 - protein, 712 aa, Artificial Sequence
  ScSED1.481 IgG1 Fc N297A mutein (without signal sequence) (ScSED1.481 Anchor)
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGVDQFSNSTSASSTDATSSSSISTSSGSVTITSSEAPESDNGTSTAAPTETSTEAPTTAIPTNGTSTEAPTDTTTEAPTTALPTNGTSTEAPTDTTTEAPTTALPTNGTSTEAPTDTTTEAPTTALPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 31 - DNA, 2229 bp, Artificial Sequence
  DNA encoding ScSED1.481 IgG1 Fc N297A mutein (with 1-19 S. cerevisiae pre-pro alpha-mating factor signal sequence) (ScSED1.481 Anchor)
atgagatttccttcaatttttactgctgttttattcgcagcatcctccgcattagctgacaagacacatacttgtccaccatgtccagctccagaattgttgggtggtccatccgttttcttgttcccaccaaagccaaaggacactttgatgatctccagaactccagaggttacatgtgttgttgttgacgtttctcacgaggacccagaggttaagttcaactggtacgttgacggtgttgaagttcacaacgctaagactaagccaagagaagagcagtacgcttccacttacagagttgtttccgttttgactgttttgcaccaggactggttgaacggtaaagaatacaagtgtaaggtttccaacaaggctttgccagctccaatcgaaaagactatctccaaggctaagggtcaaccaagagagccacaggtttacactttgccaccatccagagaagagatgactaagaaccaggtttccttgacttgtttggttaaaggattctacccatccgacattgctgttgagtgggaatctaacggtcaaccagagaacaactacaagactactccaccagttttggattctgatggttccttcttcttgtactccaagttgactgttgacaagtccagatggcaacagggtaacgttttctcctgttccgttatgcatgaggctttgcacaaccactacactcaaaagtccttgtctttgtcccctaaagctagcggtggtggtggttctgaagcagctgcaaaagaagctgctgctaagcaattttctaattctacttctgcatcatcaacagacgctacttcttcttcttctatttctacttcttctggttctgttactattacttcttctgaggcaccagagtctgacaacggtacatctactgctgctccaacagagacatccaccgaagcaccaactactgctatacccactaatggtacttctactgaagctccaactgatacaactactgaggctcctaccaccgccttgccaacaaacggtacttcaactgaggctccaacagacacaaccactgaagcacctactacagctttgccaactaacggtacctctacagaggccccaactgacactacaactgaggcacccactactgccttaccaactaatggaactacttctgctttcccaccaactacttctttgccaccatctaacactactacaacaccaccatataacccatccactgattacactactgactacactgttgtcactgaatacacaacctactgtcccgaaccaactacttttactaccaatggtaaaacatacacagttaccgaaccaacaacattaaccattactgactgtccatgcactattgagaagccaaccactacctccacaaccgaatacactgttgttactgaatacactacttactgccccgaacctacaacatttactacaaacggcaaaacttatacagtcacagagccaactactttaaccattacagactgtccttgtactatcgaaaagcctacaacaacttctacaaccgaatataccgtcgtcaccgaatatacaacttattgcccagaacccaccactttcacaaccaatggtaagacttataccgttactgagcctaccactttgactattactgattgcccatgtacaattgaaaaacctactactacttctactactgagtatacagtcgtcaccgagtatactacttattgtcccgaacctactactttcaccactaatggcaaaacctatactgttaccgagcccacaactttaactataactgattgcccttgcactattgaaaaaccaacaactacttctaccactgagtatactgtcgttactgagtacaccacatattgtcccgaacctacaacctttactactaacggcaagacatacactgttacagagcccactactttaactatcaccgattgtccatgcaccatcgaaaaatctgaagctccagagtcttctgtcccagttacagaatctaaaggtactactacaaaggaaactggtgttactactaaacaaactactgctaatccttctttgacagtttctacagttgttccagtctcttcttctgcttcttctcattctgttgttattaattctaacggtgcaaatgtcgttgttcccggtgctttgggtttggctggagttgctatgttgtttttg

SEQ ID NO: 32 - DNA, 1404 bp, Artificial Sequence
  DNA encoding adalimumab VH and an IgG1 Fc comprising the N297A mutation (with 1-19 S. cerevisiae pre-pro alpha-mating factor signal sequence)
agattcccatccatcttcactgctgttttgttcgctgcttcttctgctttggctgaagttcaattggttgaatctggtggtggtttggttcagcccggtaggtctttgaggttgtcttgcgctgcatctggtttcactttcgacgactacgctatgcattgggtcagacaagctcccggtaagggtttggaatgggtttctgctattacttggaactctggtcatattgattatgctgattctgttgaaggtagatttactatttctagagataatgctaaaaactctttgtacttgcagatgaactctttgagggctgaggacactgctgtctactactgtgctaaggtttcttatttgtctactgcttcttctttggactactggggtcaaggtactttggttactgtctcgagtgcttcaactaagggaccatctgtcttcccattggctccatcttcaaagtctacttcaggtggtactgctgctttgggttgcttggtcaaagactacttcccagagccagtcacagtttcttggaactctggtgctttgacttctggtgtccacactttcccagcagtcttacaatcttctggtttgtattcattgtcttctgttgttacagttccatcttcttctttgggtactcaaacttatatttgtaatgttaaccataaaccatctaatactaaagttgataagaaagttgaaccaaaatcttgtgataagactcacacttgtccaccttgcccagctccagaattgttaggtggtccttctgtcttcttgttcccaccaaagccaaaagatacattgatgatttctagaactccagaggttacatgtgtcgtcgtcgatgtctctcacgaggatcctgaggtcaagttcaactggtacgtcgacggtgttgaggtccacaacgctaagactaagccaagagaagaacaatacgcttctacatatagagttgtctctgttttgactgttttgcatcaggattggttaaatggtaaagaatataagtgtaaagtttctaataaggctttaccagcaccaattgaaaaaactatttctaaggctaagggtcaaccaagggaaccacaggtctacacattgccaccatcaagagaagaaatgactaaaaatcaagtttctttaacttgcttggttaagggtttctatccatcagatattgctgtcgagtgggaatctaatggtcagccagaaaataattataaaacaactccaccagttttggattctgatggttctttctttttatattctaaattgacagtcgataagtctaggtggcagcaaggtaacgttttctcatgctctgtcatgcacgaggctttgcacaaccactacacacaaaaatctttgtcattatctccaggt

SEQ ID NO: 33 - DNA, 699 bp, Artificial Sequence
  DNA encoding adalimumab VL - Kappa (with 1-19 S. cerevisiae pre-pro alpha-mating factor signal sequence)
atgagattcccatccatcttcaccgccgttttgttcgctgcttcttccgctttggctgatattcaaatgactcaatctccatcttctttgtctgcttctgtcggtgatcgtgtcactattacttgtcgtgcttctcaaggtattagaaactacttggcttggtatcaacagaagcccggtaaggctccaaagttgttgatttacgctgcttctactttgcagtctggtgtcccatctaggttctctggttctggttctggtactgattttactttgactatttcttctttgcaaccagaagatgttgctacttattattgtcaaagatataatagagctccatatacttttggtcaaggtaccaaggttgagatcaagagaactgttgctgctccatccgttttcattttcccaccatccgacgaacagttgaagtctggtacagcttccgttgtttgtttgttgaacaacttctacccaagagaggctaaggttcagtggaaggttgacaacgctttgcaatccggtaactcccaagaatccgttactgagcaagactctaaggactccacttactccttgtcctccactttgactttgtccaaggctgattacgagaagcacaaggtttacgcttgtgaggttacacatcagggtttgtcctccccagttactaagtccttcaacagaggagagtgt

SEQ ID NO: 34 - protein, 680 aa, Artificial Sequence
  SED1-FLO5-680 without signal sequence
QFSNSTSASSTDVTSSSSISTSSGSVTITSSEAPESDNGTSTAAPTETSTEAPTTAIPTNGTSTEAPTTAIPTNGTSTEAPTDTTTEAPTTALPTNGTSTEAPTDTTTEAPTTGLPTNGTTTEPWTGTFTSTSTEMTTITDTNGQLTDETVIVIRTPTTASTITTTTEPWTGTFTSTSTEMTTVTGTNGQPTDETVIVIRTPTSEGLITTTTEPWTGTFTSTSTEMTTVTGTNGQPTDETVIVIRTPTSEGLITTTTEPWTGTFTSTSTEVTTITGTNGQPTDETVIVIRTPTSEGLITTTTEPWTGTFTSTSTEMTTVTGTNGQPTDETVIVIRTPTSEGLISTTTEPWTGTFTSTSTEVTTITGTNGQPTDETVIVIRTPTSEGLITTTTEPWTGTFTSTSTEMTTVTGTNGQPTDETVIVIRTPTSEGLITRTTEPWTGTFTSTSTEVTTITGTNGQPTDETVIVIRTPTTAISSSTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 35 - protein, 660 aa, Artificial Sequence
  SED1-FLO1-660 without signal sequence
QFSNSTSASSTDVTSSSSISTSSGSVTITSSEAPESDNGTSTAAPTETSTEAPTTAIPTNGTSTEAPTTAIPTNGTSTEAPTDTTTEAPTTALPTNGTSTEAPTDTTTEAPTTGLPTNGTTTEPWTGTFTSTSTEMTTITGTNGVPTDETVIVIRTPTSEGLISTTTEPWTGTFTSTSTEMTTITGTNGQPTDETVIVIRTPTSEGLISTTTEPWTGTFTSTSTEMTHVTGTNGVPTDETVIVIRTPTSEGLISTTTEPWTGTFTSTSTEVTTITGTNGQPTDETVIVIRTPTSEGLISTTTEPWTGTFTSTSTEMTTVTGTNGQPTDETVIVIRTPTSEGLVTTTTEPWTGTFTSTSTEMSTVTGTNGLPTDETVIVVKTPTTAISSSLSSSSSGQITSSITSSRPIITPFYPSNGTSVISSSVISSSVTSSLFTSSPVISSSVISSSTTTSTSIFSETTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 36 - DNA, 2826 bp, Artificial Sequence
  DNA encoding SED1-FLO5-680 fusion anchor (IgG1 (N297A) with 1-19 S. cerevisiae pre-pro alpha-mating factor signal sequence and linker)
atgagatttccttcaatttttactgctgttttattcgcagcatcctccgcattagctgacaagacacatacttgtccaccatgtccagctccagaattgttgggtggtccatccgttttcttgttcccaccaaagccaaaggacactttgatgatctccagaactccagaggttacatgtgttgttgttgacgtttctcacgaggacccagaggttaagttcaactggtacgttgacggtgttgaagttcacaacgctaagactaagccaagagaagagcagtacgcttccacttacagagttgtttccgttttgactgttttgcaccaggactggttgaacggtaaagaatacaagtgtaaggtttccaacaaggctttgccagctccaatcgaaaagactatctccaaggctaagggtcaaccaagagagccacaggtttacactttgccaccatccagagaagagatgactaagaaccaggtttccttgacttgtttggttaaaggattctacccatccgacattgctgttgagtgggaatctaacggtcaaccagagaacaactacaagactactccaccagttttggattctgatggttccttcttcttgtactccaagttgactgttgacaagtccagatggcaacagggtaacgttttctcctgttccgttatgcatgaggctttgcacaaccactacactcaaaagtccttgtctttgtcccctaaagctagcggaggtggaggttctgaagccgcggcgaaagaggcagctgccaaacagtttagcaatagtacgagcgcctcctcaaccgatgtgacgtcatcaagctccattagtacaagtagtggcagcgtcacaataactagtagcgaggcacccgagtcagacaatggcaccagtacagcagctcctaccgagactagtacagaggccccgaccacagctatcccaaccaatggcacatccaccgaagcccccactacggcgattcccacgaacggtacgtccacggaagctcccacagacaccactacagaggcgcctacgaccgccctacctactaatgggaccagtacggaagccccaacggatactaccacagaggcacccacgacgggactaccaacaaacggcactaccacggagccgtggaccggtacctttacgagcacgtctaccgaaatgactactatcacggacaccaatggtcaacttacggatgagacggtgattgtgataagaactccaactaccgcaagcactatcaccacaaccacggaaccttggactggaactttcacatctacctccaccgagatgactaccgtaacgggcacgaatggacaaccgactgacgaaactgtaatagtcatccgtacgcccacgtcagagggcttaattacaactacaaccgagccgtggacgggaacattcaccagtacaagcacagagatgaccacggtaaccggcacaaacgggcagcctacagatgagacagtaatcgttatacgtacacccacctccgaaggattgataactactacaaccgaaccctggacgggaaccttcacctctacgagtactgaagtaacgaccataactgggacaaatggccaacctactgatgaaacagtcatcgtaatcaggaccccgaccagcgagggtttgattacgaccaccacggagccatggacaggaacttttactagcacaagtaccgaaatgactactgtcactggaactaacggccagccgactgacgagacggtaattgtaattaggacccccactagtgagggtctaataagtacaactactgaaccttggacggggacgttcacatccacgagcacagaggtcaccacaattacagggaccaacggacagccaactgatgagactgtgatcgttattaggaccccaacatctgaagggttaatcaccacgaccaccgagccgtggacaggtacatttacctctaccagcacagagatgacaactgtaacaggtactaatggacaaccgactgacgagacagtcatcgtcatccgaacccccacgagtgaaggcttgattacgcgaacaacggaaccctggaccggcaccttcacaagtacgtcaacagaggtcactacgattacgggaactaatgggcagccaactgatgagacagtcatcgtaattcggacgccgaccacggccatcagcagcagtacgacctctgcattcccccctactaccagcttgccaccgagcaatactactactactcccccgtataaccctagtacagattataccacagattacactgtagttaccgagtacaccacatactgtcccgaaccaactacgtttaccaccaatgggaaaacgtacaccgtgaccgaaccgacaactctcactattactgactgcccatgtactattgagaagcccaccacgacttcaaccactgaatataccgtagtgactgaatatacaacctactgccctgaacctacaacattcaccactaatggcaagacttacaccgtgacggaacctacaacattgacaattaccgactgtccttgcactatcgagaaatctgaggccccagaaagttctgtcccagttactgaatccaaggggacgacaactaaggagactggggtaacgaccaagcagacaactgccaacccaagtctcaccgtaagcactgtggtaccggtaagtagttcagcgagtagtcactctgttgtaatcaactccaatggcgccaacgttgtagtaccgggggcacttggtcttgctggggttgcgatgctgttcttg

SEQ ID NO: 37 - protein, 942 aa, Artificial Sequence
  SED1-FLO5-680 fusion anchor (IgG1 (N297A) with 1-19 S. cerevisiae pre-pro alpha-mating factor signal sequence and linker)
MRFPSIFTAVLFAASSALADKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPKASGGGGSEAAAKEAAAKQFSNSTSASSTDVTSSSSISTSSGSVTITSSEAPESDNGTSTAAPTETSTEAPTTAIPTNGTSTEAPTTAIPTNGTSTEAPTDTTTEAPTTALPTNGTSTEAPTDTTTEAPTTGLPTNGTTTEPWTGTFTSTSTEMTTITDTNGQLTDETVIVIRTPTTASTITTTTEPWTGTFTSTSTEMTTVTGTNGQPTDETVIVIRTPTSEGLITTTTEPWTGTFTSTSTEMTTVTGTNGQPTDETVIVIRTPTSEGLITTTTEPWTGTFTSTSTEVTTITGTNGQPTDETVIVIRTPTSEGLITTTTEPWTGTFTSTSTEMTTVTGTNGQPTDETVIVIRTPTSEGLISTTTEPWTGTFTSTSTEVTTITGTNGQPTDETVIVIRTPTSEGLITTTTEPWTGTFTSTSTEMTTVTGTNGQPTDETVIVIRTPTSEGLITRTTEPWTGTFTSTSTEVTTITGTNGQPTDETVIVIRTPTTAISSSTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 38 - protein, 923 aa, Artificial Sequence
  SED1-FLO5-680 fusion anchor (IgG1 (N297A)
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPKASGGGGSEAAAKEAAAKQFSNSTSASSTDVTSSSSISTSSGSVTITSSEAPESDNGTSTAAPTETSTEAPTTAIPTNGTSTEAPTTAIPTNGTSTEAPTDTTTEAPTTALPTNGTSTEAPTDTTTEAPTTGLPTNGTTTEPWTGTFTSTSTEMTTITDTNGQLTDETVIVIRTPTTASTITTTTEPWTGTFTSTSTEMTTVTGTNGQPTDETVIVIRTPTSEGLITTTTEPWTGTFTSTSTEMTTVTGTNGQPTDETVIVIRTPTSEGLITTTTEPWTGTFTSTSTEVTTITGTNGQPTDETVIVIRTPTSEGLITTTTEPWTGTFTSTSTEMTTVTGTNGQPTDETVIVIRTPTSEGLISTTTEPWTGTFTSTSTEVTTITGTNGQPTDETVIVIRTPTSEGLITTTTEPWTGTFTSTSTEMTTVTGTNGQPTDETVIVIRTPTSEGLITRTTEPWTGTFTSTSTEVTTITGTNGQPTDETVIVIRTPTTAISSSTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 39 - DNA, 2766 bp, Artificial Sequence
  DNA encoding SED1-FLO1-660 fusion anchor (with 1-19 S. cerevisiae pre-pro alpha-mating factor signal sequence)
atgagatttccttcaatttttactgctgttttattcgcagcatcctccgcattagctgacaagacacatacttgtccaccatgtccagctccagaattgttgggtggtccatccgttttcttgttcccaccaaagccaaaggacactttgatgatctccagaactccagaggttacatgtgttgttgttgacgtttctcacgaggacccagaggttaagttcaactggtacgttgacggtgttgaagttcacaacgctaagactaagccaagagaagagcagtacgcttccacttacagagttgtttccgttttgactgttttgcaccaggactggttgaacggtaaagaatacaagtgtaaggtttccaacaaggctttgccagctccaatcgaaaagactatctccaaggctaagggtcaaccaagagagccacaggtttacactttgccaccatccagagaagagatgactaagaaccaggtttccttgacttgtttggttaaaggattctacccatccgacattgctgttgagtgggaatctaacggtcaaccagagaacaactacaagactactccaccagttttggattctgatggttccttcttcttgtactccaagttgactgttgacaagtccagatggcaacagggtaacgttttctcctgttccgttatgcatgaggctttgcacaaccactacactcaaaagtccttgtctttgtcccctaaagctagcggaggaggtggaagcgaagctgccgctaaggaggcagccgccaagcagttttctaattccacgagcgcgtctagtaccgacgtgacatctagttcatccatcagtactagcagcggctccgtcactataactagctctgaggcacccgagagcgacaatggtacatcaacagctgcccctacagaaacttctacggaggcccccacgaccgccatacctaccaatggcaccagtactgaggcgcctactaccgcaatcccgacaaacggaaccagcacagaagctccaactgatacaacaacagaagccccaacaacagcactgccgacgaacggcacgtctaccgaagctcccacggacacgacaactgaggcgccgactacaggattacccacaaatgggactaccaccgagccttggacagggaccttcacctctacgtctaccgaaatgacaacaataaccggtacgaacggagtaccaactgacgagactgtcattgttattcgtacaccaacaagcgaaggtcttataagcacgacgactgagccatggactgggacttttacgagtacgagtacggagatgactactataacaggcaccaatggccagcccactgacgagaccgtcatagttataaggactcctacctcagaaggattaatctctacaacaacagagccatggacgggcaccttcacttcaacgtcaactgagatgacacatgtgacaggtacgaatggtgtaccaaccgacgagacggtcatcgtgattaggactcccacaagcgaggggctgatatcaacaactactgaaccctggacgggtacgttcactagtacatccaccgaagttacgactataaccggaacgaacgggcaacccactgatgaaaccgttattgtgatcaggacgcctacttccgaaggtctgatatctaccacgactgagccctggacgggcacgttcacttcaacttccactgaaatgacgacggtgactggaaccaacggtcaaccgaccgacgaaaccgtcatagtgattaggacacctaccagtgagggactagttaccactaccacggaaccatggactggtacatttacatcaacatccaccgagatgtctacagttacgggcacaaatggcttgcctacagatgaaaccgtgatagttgttaaaacgcccaccactgcaatttcaagcagcttgtctagctctagctccggacaaattaccagttccataacatctagccgtccaattataacaccattttatccaagcaacggcactagtgtgatttcctccagtgttatatcatctagtgttacgtcatcactattcacatcatcccctgtcatctcttcatccgttatctcctcttctacgaccacgtcaacgtcaatttttagtgaaacaacctctgcatttcccccgacgacgtctctacctccaagtaatacgactactactcccccttataatccgagcacagactacacaactgactataccgtcgttacggagtacacgacgtattgccctgaacccacaacctttactactaatgggaaaacttacactgtcactgaaccgaccacgctaacgattacagactgcccctgcacgatagaaaaacctaccaccacttcaaccacggagtacactgtggtgactgaatacacgacatactgtccagaacctacaaccttcacaaccaacggaaaaacatacacggtgacggaacctacaacgctgacgatcactgattgcccatgtacaatcgagaaatctgaagctccagaatcatctgtcccggttactgaatcaaagggtactacaaccaaggaaactggggtcactacaaaacaaaccacggcaaatccgagtttgacagtcagtacggtagtgcctgtgtctagtagcgccagttcacacagcgtagtgataaattctaatggggcgaacgtggttgtacctggcgcgttaggcttggccggggtagccatgctttttttg

SEQ ID NO: 40 - protein, 922 aa, Artificial Sequence
  SED1-FLO1-660 fusion anchor (with 1-19 S. cerevisiae pre-pro alpha-mating factor signal sequence)
MRFPSIFTAVLFAASSALADKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPKASGGGGSEAAAKEAAAKQFSNSTSASSTDVTSSSSISTSSGSVTITSSEAPESDNGTSTAAPTETSTEAPTTAIPTNGTSTEAPTTAIPTNGTSTEAPTDTTTEAPTTALPTNGTSTEAPTDTTTEAPTTGLPTNGTTTEPWTGTFTSTSTEMTTITGTNGVPTDETVIVIRTPTSEGLISTTTEPWTGTFTSTSTEMTTITGTNGQPTDETVIVIRTPTSEGLISTTTEPWTGTFTSTSTEMTHVTGTNGVPTDETVIVIRTPTSEGLISTTTEPWTGTFTSTSTEVTTITGTNGQPTDETVIVIRTPTSEGLISTTTEPWTGTFTSTSTEMTTVTGTNGQPTDETVIVIRTPTSEGLVTTTTEPWTGTFTSTSTEMSTVTGTNGLPTDETVIVVKTPTTAISSSLSSSSSGQITSSITSSRPIITPFYPSNGTSVISSSVISSSVTSSLFTSSPVISSSVISSSTTTSTSIFSETTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 41 - protein, 903 aa, Artificial Sequence
  SED1-FLO1-660 fusion anchor
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPKASGGGGSEAAAKEAAAKQFSNSTSASSTDVTSSSSISTSSGSVTITSSEAPESDNGTSTAAPTETSTEAPTTAIPTNGTSTEAPTTAIPTNGTSTEAPTDTTTEAPTTALPTNGTSTEAPTDTTTEAPTTGLPTNGTTTEPWTGTFTSTSTEMTTITGTNGVPTDETVIVIRTPTSEGLISTTTEPWTGTFTSTSTEMTTITGTNGQPTDETVIVIRTPTSEGLISTTTEPWTGTFTSTSTEMTHVTGTNGVPTDETVIVIRTPTSEGLISTTTEPWTGTFTSTSTEVTTITGTNGQPTDETVIVIRTPTSEGLISTTTEPWTGTFTSTSTEMTTVTGTNGQPTDETVIVIRTPTSEGLVTTTTEPWTGTFTSTSTEMSTVTGTNGLPTDETVIVVKTPTTAISSSLSSSSSGQITSSITSSRPIITPFYPSNGTSVISSSVISSSVTSSLFTSSPVISSSVISSSTTTSTSIFSETTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 42 - protein, 522 aa, Artificial Sequence
  S. cerevisiae PDI1 (amino acids 1-28 signal sequence)
MKFSAGAVLSWSSLLLASSVFAQQEAVAPEDSAVVKLATDSFNEYIQSHDLVLAEFFAPWCGHCKNMAPEYVKAAETLVEKNITLAQIDCTENQDLCMEHNIPGFPSLKIFKNSDVNNSIDYEGPRTAEAIVQFMIKQSQPAVAVVADLPAYLANETFVTPVIVQSGKIDADFNATFYSMANKHFNDYDFVSAENADDDFKLSIYLPSAMDEPVVYNGKKADIADADVFEKWLQVEALPYFGEIDGSVFAQYVESGLPLGYLFYNDEEELEEYKPLFTELAKKNRGLMNFVSIDARKFGRHAGNLNMKEQFPLFAIHDMTEDLKYGLPQLSEEAFDELSDKIVLESKAIESLVKDFLKGDASPIVKSQEIFENQDSSVFQLVGKNHDEIVNDPKKDVLVLYYAPWCGHCKRLAPTYQELADTYANATSDVLIAKLDHTENDVRGVVIEGYPTIVLYPGGKKSESVVYQGSRSLDSLFDFIKENGHFDVDGKALYEEAQEKAAEEADADAELADEEDAIHDEL

(19) United States
(12) Patent Application Publication
(10) Pub. No.: US 2024/0011016 A1
(43) Pub. Date: Jan. 11, 2024

(54) FULL-LENGTH HUMAN IMMUNOGLOBULIN G ANTIBODY LIBRARIES FOR SURFACE DISPLAY AND SECRETION IN SACCHAROMYCES CEREVISIAE

(71) Applicant: MERCK SHARP & DOHME LLC, Rahway, NJ (US)

(72) Inventors: Michelle Castor, Jamaica Plain, MA (US); Ming-Tang Chen, Dover, MA (US); Chung-Ming Hsieh, Newton, MA (US)

(73) Assignee: MERCK SHARP & DOHME LLC, Rahway, NJ (US)

(21) Appl. No.: 18/252,271

(22) PCT Filed: Nov. 18, 2021
(86) PCT No.: PCT/US2021/059814

§ 371 Date: May 9, 2023

Related U.S. Application Data
(60) Provisional application No. 63/117,289, filed on Nov. 23, 2020.

(51) Int. Cl. C12N 15/10; C07K 16/30; C07K 16/24

(52) U.S. Cl. CPC C12N 15/1037; C12N 15/1055; C07K 16/3015; C07K 16/241; C07K 2317/522; C07K 2317/35

(57) ABSTRACT

A Saccharomyces cerevisiae antibody display system that simultaneously secrets and displays antibody fragments from a very large size synthetic nave human antibody library for therapeutic antibody lead discovery and engineering is described. A bait anchor complexed with a monovalent antibody fragment is tethered on the surface of the S. cerevisiae host cell, wherein the fragment can be assayed for antigen binding, while the full bivalent antibody is simultaneously secreted from the host cell. Methods of using the system for identifying antibodies from the library that bind specifically to an antigen of interest are also provided. Polypeptides, polynucleotides and host cells used for making the antibody display system are also provided along with methods of use thereof.

### BACKGROUND OF THE INVENTION

### (1) Field of the Invention

The field of the invention relates to Saccharomyces cerevisiae recombinant protein and antibody surface display systems, recombinant antibody libraries, and methods of use for identifying recombinant antibodies that bind specifically to an antigen.

### (2) Description of Related Art

The discovery of monoclonal antibodies has evolved from hybridoma technology for producing the antibodies to direct selection of antibodies from human cDNA or synthetic DNA libraries. This has been driven in part by the desire to engineer improvements in binding affinity and specificity of the antibodies to improve efficacy of the antibodies. Thus, combinatorial library screening and selection methods have become a common tool for altering the recognition properties of proteins (Ellman et al., Proc. Natl. Acad. Sci. USA 94: 2779-2782 (1997): Phizicky & Fields. Microbiol. Rev, 59: 94-123 (1995)). The ability to construct and screen antibody libraries in vitro promises improved control over the strength and specificity of antibody-antigen interactions.

The most widespread technique for constructing and screening antibody libraries is phage display, whereby the protein of interest is expressed as a polypeptide fusion to a bacteriophage coat protein and subsequently screened by binding to immobilized or soluble biotinylated ligand. Fusions are made most commonly to a minor coat protein, called the gene III protein (pill), which is present in three to five copies at the tip of the phage. A phage constructed in this way can be considered a compact genetic “unit”, possessing both the phenotype (binding activity of the displayed antibody) and genotype (the gene coding for that antibody) in one package. Phage display has been successfully applied to antibodies, DNA binding proteins, protease inhibitors, short peptides, and enzymes (Choo & Klug, Curr. Opin. Biotechnol. 6: 431-436 (1995); Hoogenboom, Trends Biotechnol. 15: 62-70 (1997); Ladner, Trends Biotechnol. 13: 426-430 (1995); Lowman et al., Biochemistry 30: 10832-10838 (1991); Markland et al., Methods Enzymol. 267: 28-51 (1996); Matthews & Wells, Science 260: 1113-1117 (1993); Wang et al., Methods Enzymol. 267: 52-68 (1996)).

Antibodies possessing desirable binding properties are selected by binding to immobilized antigen in a process called “panning”. Phage bearing nonspecific antibodies are removed by washing, and then the bound phage are eluted and amplified by infection of E. coli. This approach has been applied to generate antibodies against many antigens.

Nevertheless, phage display possesses several shortcomings. Although panning of antibody phage display libraries is a powerful technology, it possesses several intrinsic difficulties that limit its wide-spread successful application. For example, some eukaryotic secreted proteins and cell surface proteins require post-translational modifications such as glycosylation or extensive disulfide isomerization, which are unavailable in bacterial cells. Furthermore, the nature of phage display precludes quantitative and direct discrimination of ligand binding parameters. For example, very high affinity antibodies (Kd≤1 nM) are difficult to isolate by panning, since the elution conditions required to break a very strong antibody-antigen interaction are generally harsh enough (e.g., low pH, high salt) to denature the phage particle sufficiently to render it non-infective.

Additionally, the requirement for physical immobilization of an antigen to a solid surface produces many artifactual difficulties. For example, high antigen surface density introduces avidity effects which mask true affinity. Also, physical tethering reduces the translational and rotational entropy of the antigen, resulting in a smaller ΔS upon antibody binding and a resultant overestimate of binding affinity relative to that for soluble antigen and large effects from variability in mixing and washing procedures lead to difficulties with reproducibility. Furthermore, the presence of only one to a few antibodies per phage particle introduces substantial stochastic variation, and discrimination between antibodies of similar affinity becomes impossible. For example, affinity differences of six-fold or greater are often required for efficient discrimination (Riechmann & Weill, Biochem. 32, 8848-55 (1993)). Finally, populations can be overtaken by more rapidly growing wild-type phage. In particular, since pIII is involved directly in the phage life cycle, the presence of some antibodies or bound antigens will prevent or retard amplification of the associated phage.

Additional bacterial cell surface display methods have been developed (Francisco, et al., Proc. Natl. Acad. Sci. USA 90: 10444-10448 (1993); Georgiou et al., Nat. Biotechnol. 15: 29-34 (1997)). However, use of a prokaryotic expression system occasionally introduces unpredictable expression biases (Knappik & Pluckthun, Prot. Eng. 8: 81-89 (1995); Ulrich et al., Proc. Natl. Acad. Sci. USA 92: 11907-11911 (1995); Walker & Gilbert, J. Biol. Chem 269: 28487-28493 (1994)) and bacterial capsular polysaccharide layers present a diffusion barrier that restricts such systems to small molecule ligands (Roberts. Annu. Rev. Microbiol. 50: 285-315 (1996)). E. coli possesses a lipopolysaccharide layer or capsule that may interfere sterically with macromolecular binding reactions. In fact, a presumed physiological function of the bacterial capsule is restriction of macromolecular diffusion to the cell membrane, in order to shield the cell from the immune system (DiRienzo et al., Ann. Rev. Biochem. 47: 481-532, (1978)). Since the periplasm of E. coli has not evolved as a compartment for the folding and assembly of antibody fragments, expression of antibodies in E. coli has typically been very clone dependent, with some clones expressing well and others not at all. Such variability introduces concerns about equivalent representation of all possible sequences in an antibody library expressed on the surface of E. coli. Moreover, phage display does not allow some important posttranslational modifications such as glycosylation that can affect specificity or affinity of the antibody. About a third of circulating monoclonal antibodies contain one or more N-linked glycans in the variable regions. In some cases it is believed that these N-glycans in the variable region may play a significant role in antibody function.

The efficient production of monoclonal antibody therapeutics would be facilitated by the development of alternative test systems that utilize lower eukaryotic cells, such as yeast cells. The structural similarities between B-cells displaying antibodies and yeast cells displaying antibodies provide a closer analogy to in vivo affinity maturation than is available with filamentous phage. In particular, because lower eukaryotic cells are able to produce glycosylated proteins, whereas filamentous phage cannot, monoclonal antibodies produced in lower eukaryotic host cells are more likely to exhibit similar activity in humans and other mammals as they do in test systems which utilize lower eukaryotic host cells.

Moreover, the ease of growth culture and facility of genetic manipulation available with yeast will enable large populations to be mutagenized and screened rapidly. By contrast with conditions in the mammalian body, the physicochemical conditions of binding and selection can be altered for a yeast culture within a broad range of pH, temperature, and ionic strength to provide additional degrees of freedom in antibody engineering experiments. The development of yeast surface display system for screening combinatorial protein libraries has been described.

U.S. Pat. Nos. 6,300,065 and 6,699,658 describe the development of a yeast surface display system for screening combinatorial antibody libraries and a screen based on antibody-antigen dissociation kinetics. The system relies on transfecting yeast with vectors that express an antibody or antibody fragment fused to a yeast cell wall protein, using mutagenesis to produce a variegated population of mutants of the antibody or antibody fragment and then screening and selecting those cells that produce the antibody or antibody fragment with the desired enhanced phenotypic properties. U.S. Pat. No. 7,132,273 discloses various yeast cell wall anchor proteins and a surface expression system that uses them to immobilize foreign enzymes or polypeptides on the cell wall.

Of interest are Tanino et al, Biotechnol. Prog. 22: 989-993 (2006), which discloses construction of a Pichia pastoris cell surface display system using Flo1p anchor system; Ren et al., Molec. Biotechnol. 35:103-108 (2007), which discloses the display of adenoregulin in a Pichia pastoris cell surface display system using the Flo1p anchor system; Mergler et al., Appl. Microbiol. Biotechnol. 63:418-421 (2004), which discloses display of K. lactis yellow enzyme fused to the C-terminus half of S. cerevisiae α-agglutinin; Jacobs et al., Abstract T23, Pichia Protein expression Conference, San Diego, CA (Oct. 8-11, 2006), which discloses display of proteins on the surface of Pichia pastoris using α-agglutinin: Ryckaert et al., Abstracts BVBMB Meeting, Vrije Universiteit Brussel, Belgium (Dec. 2, 2005), which discloses using a yeast display system to identify proteins that bind particular lectins; U.S. Pat. No. 7,166,423, which discloses a method for identifying cells based on the product secreted by the cells by coupling to the cell surface a capture moiety that binds the secreted product, which can then be identified using a detection means: U.S. Published Application No. 2004/0219611, which discloses a biotin-avidin system for attaching protein A or G to the surface of a cell for identifying cells that express particular antibodies; U.S. Pat. No. 6,919,183, which discloses a method for identifying cells that express a particular protein by expressing in the cell a surface capture moiety and the protein wherein the capture moiety and the protein form a complex which is displayed on the surface of the cell; U.S. Pat. No. 6,114,147, which discloses a method for immobilizing proteins on the surface of a yeast or fungal using a fusion protein consisting of a binding protein fused to a cell wall protein which is expressed in the cell.

U.S. Pat. Nos. 8,067,339 and 9,260,712 disclose an improvement to yeast surface display system in which the capture and display of whole antibodies suitable in yeast, particularly. Pichia pastoris, is achieved by sequential expression of the capture moiety fused to a cell surface anchor protein followed by inhibition of expression of the capture moiety and subsequent expression of the antibody heavy and light chains. A further improvement is disclosed in U.S. Pat. Nos. 9,365,846 and 10,106,598, which disclose a yeast surface display system that uses an antibody Fc tethered to the cell surface by a cell surface anchor protein to serve as bait for capturing and displaying on the cell surface an antibody heavy chain/light chain pair paired with the antibody Fc. Another improvement is disclosed in U.S. Pat. Nos. 9,890,378 and 10,577,600, which disclose a yeast surface display system that uses an antibody light chain tethered to the cell surface by a cell surface anchor protein to serve as bait for capturing and displaying on the cell surface an antibody in which one of the two light chains is provided by the bait.

The potential applications of engineering antibodies for the diagnosis and treatment of all manner of human disease such as cancer therapy, tumor imaging, sepsis are far-reaching. For these applications, antibodies with high affinity (i.e., Kd≤10 nM) and high specificity are highly desirable. Anecdotal evidence, as well as the a priori considerations discussed previously, suggests that phage display or bacterial display systems are unlikely to consistently produce antibodies of sub-nanomolar affinity. Also, antibodies identified using phage display or bacterial display systems may not be susceptible to commercial scale production in eukaryotic cells. Therefore, development of further protein expression systems based on improved vectors and host cell lines in which effective protein display facilitates development of genetically enhanced cells for recombinant production of immunoglobulins is a desirable objective.

### BRIEF SUMMARY OF THE INVENTION

The present invention provides an improved yeast antibody display system using Saccharomyces cerevisiae as the host that has increased diversity and efficiency over currently available systems using Saccharomyces cerevisiae. The system of the present invention provides a Saccharomyces cerevisiae host cell antibody library wherein each host cell displays on the cell surface either long, naturally occurring SED1 variants (for example, SED1.499) or long engineered semi-synthetic surface anchor proteins exemplified by SED1-FLO1-660, SED1-FLO1-678, and SED1-FLO5-680, either fused to an antibody Fc domain. The displayed Fc domain is capable of binding to functionally active “half” IgGs (monovalent antibodies) produced by the host cell, which are displayed on the cell wall of the host cell. Host cells that display a “half” antibody on the cell surface can be identified by screening cells in the host library with a labeled antigen recognized by the “half” IgG and isolating said cells by conventional cell sorting methods.

The present invention further provides an efficient yeast transformation protocol that enables up to 10×10⁹ yeast transformant cells to be routinely achieved in one day, e.g., constructions of greater than 10×10⁹ IgG heavy chain yeast libraries and greater than 10×10⁹ light chain yeast libraries. By mating greater than 10×10⁹ heavy chain yeast library cells with greater than 10×10⁹ light chain yeast library cells, very large combinational heavy×light antibody display libraries may be achieved.

The present invention provides an antibody display system comprising (a) an isolated yeast host cell; (b) a polynucleotide encoding a bait comprising (i) an immunoglobulin heavy chain Fc domain fused to (ii) a cell surface anchor polypeptide comprising more than 320 amino acids, operably linked to a regulatable promotor; (c) one or more polynucleotides encoding an immunoglobulin light chain variable domain (VL); and (d) one or more polynucleotides encoding an immunoglobulin heavy chain variable domain (VH).

In a further embodiment of the antibody display system, the antibody display system further comprises (i) a non-tethered or secreted unbound full-length bivalent antibody tetramer comprising two immunoglobulin heavy chains (HC), each HC comprising said VH, and two immunoglobulin light chains (LC), each LC comprising said VL; and/or (ii) a monovalent antibody fragment comprising one HC and one LC complexed with or tethered to the Fc moiety of the bait.

In a further embodiment of the antibody display system, said one or more polynucleotides encoding a VL is from a diverse population of VLs; and/or, wherein said one or more polynucleotides encoding a VH is from a diverse population of VHs. In particular embodiments, the diverse population of VHs comprises at least 10⁹ VH sequences and the diverse population of VLs comprises at least 10⁹ VL sequences.

In a further embodiment of the antibody display system, the VL is fused to an immunoglobulin light chain constant domain and the VH is fused to an immunoglobulin heavy chain constant domain having an Fc domain or immunoglobulin heavy chain CH1 domain and lacking an Fc domain.

In a further embodiment of the antibody display system, the immunoglobulin heavy chain constant domain is an IgG1, IgG2, IgG3, or IgG4 immunoglobulin constant domain or the Fc immunoglobulin domain is an IgG1, IgG2, IgG3 or IgG4 Fc immunoglobulin domain. In particular embodiments, the Fc domain comprises an N297A amino acid substitution, wherein the numbering is in accordance with the Eu numbering scheme.

In a further embodiment of the antibody display system, the surface anchor polypeptide comprises between 400 to 700 amino acids.

In a further embodiment of the antibody display system, the regulatable promoter is a TetO7 promoter.

In a further embodiment of the antibody display system, the surface anchor polypeptide comprises a Saccharomyces cerevisiae SED1 protein. In particular embodiments, the surface anchor polypeptide comprises a Saccharomyces cerevisiae SED1 protein comprising about 401, 430, or 481 amino acids. In further still embodiments, the cell surface anchor polypeptide comprises a Saccharomyces cerevisiae SED1 protein comprising the amino acid sequence set forth in SEQ ID NO: 18, SEQ ID NO: 19, or SEQ ID NO: 20.

In further embodiments, the cell surface anchor polypeptide is a chimeric surface anchor polypeptide comprising a Saccharomyces cerevisiae SED1 protein and a heterologous protein. In a further embodiment, the cell surface anchor polypeptide is a chimeric surface anchor polypeptide comprising a heterologous protein amino acid sequence linked at its N-terminus to the N-terminal portion of a Saccharomyces cerevisiae SED1 protein and at its C-terminus to the C-terminal portion of a Saccharomyces cerevisiae SED1 protein. In a further embodiment, the heterologous protein amino acid sequence is a minisatellite-like repeat sequence from a yeast cell wall protein. In particular embodiments, the yeast cell wall protein is selected from FLO1, FLO2, and FLO11. In exemplary embodiments, the chimeric surface anchor polypeptide has an amino acid sequence selected from SEQ ID NO: 34 or SEQ ID NO: 35.

In particular embodiments of the antibody display system, the bait comprises an amino acid sequence set forth in SEQ ID NO: 28, SEQ ID NO: 29, SEQ ID NO: 30, SEQ ID NO: 38, or SEQ ID NO: 41.

In particular embodiments, the one or more polynucleotides encoding the VL and VH are each operably linked to a second regulatable promoter. In particular embodiments, the one or more polynucleotides encoding the LC and HC are each operably linked to a second regulatable promoter. In a further embodiment, the second regulatable promoter is a GAL1 promoter.

In further embodiments of the antibody display system, the isolated yeast is Saccharomyces cerevisiae. In particular embodiments, the isolated yeast host cell is diploid.

In further embodiments of the antibody display system, the VL and VH are human or humanized.

The present invention provides a method for the selection of a yeast diploid cell that secretes an antibody tetramer that selectively binds a molecule of interest, the method comprising: (a) transforming a multiplicity of yeast haploid cells with (i) a first polynucleotide, said first polynucleotide encoding an Fc bait polypeptide comprising the Fc domain of an antibody heavy chain constant domain fused to a cell surface anchor polypeptide, which said cell surface anchor polypeptide comprises more than 320 amino acids, operably linked to a first regulatable promoter, and (ii) a plurality of second polynucleotides, each second polynucleotide independently encoding an antibody heavy chain variable domain (VH), operably linked to a second regulatable promoter, to provide a plurality of first yeast haploid cells; (b) transforming a multiplicity yeast haploid cells with a plurality of third polynucleotides, each third polynucleotide independently encoding a light chain variable domain (VL), operably linked to the second regulatable promoter, to provide a plurality of second yeast haploid cells; (c) generating a plurality of yeast diploid cells from said first and second yeast haploid cells; (d) culturing said plurality of yeast diploid cells under a first condition wherein the Fc bait polypeptide and the antibody VH and VL are expressed and displayed on the surface of the diploid yeast cells in a complex comprising the Fc bait complexed to a monovalent antibody fragment comprising a heavy chain variable domain and a light chain variable domain; (e) selecting those yeast diploid cells in the plurality of yeast diploid cells in which the monovalent antibody fragment selectively binds the molecule of interest to provide selected yeast diploid cells; and (f) culturing at least one selected yeast diploid cell under a second condition wherein full-length bivalent antibody tetramers comprising two immunoglobulin heavy chains and two immunoglobulin light chains that specifically bind the molecule of interest are expressed and secreted from the selected yeast diploid cell and the Fc bait is not expressed.

In further embodiments of the method, the VL is fused to an immunoglobulin light chain constant domain and the VH is fused to an immunoglobulin heavy chain constant domain having an Fc domain or immunoglobulin heavy chain CH1 domain and lacking an Fc domain.

In a further embodiment of the method, the immunoglobulin heavy chain constant domain is an IgG1, IgG2, IgG3, or IgG4 immunoglobulin constant domain or the Fc immunoglobulin domain is an IgG1, IgG2, IgG3 or IgG4 Fc immunoglobulin domain. In particular embodiments, the Fc domain comprises an N297A amino acid substitution, wherein the numbering is in accordance with the Eu numbering scheme.

In a further embodiment of the method, the surface anchor polypeptide comprises between 400 to 7(0) amino acids.

In a further embodiment of the method, the regulatable promoter is a TetO7 promoter.

In a further embodiment of the method, the surface anchor polypeptide comprises a Saccharomyces cerevisiae SED1 protein. In particular embodiments, the surface anchor polypeptide comprises a Saccharomyces cerevisiae SED1 protein comprising about 401, 430, or 481 amino acids. In further still embodiments, the cell surface anchor polypeptide comprises a Saccharomyces cerevisiae SED1 protein comprising the amino acid sequence set forth in SEQ ID NO: 18, SEQ ID NO: 19, or SEQ ID NO: 20.

In further embodiments of the method, the cell surface anchor polypeptide is a chimeric surface anchor polypeptide comprising a Saccharomyces cerevisiae SED1 protein and a heterologous protein. In a further embodiment, the cell surface anchor polypeptide is a chimeric surface anchor polypeptide comprising a heterologous protein amino acid sequence linked at its N-terminus to the N-terminal portion of a Saccharomyces cerevisiae SED1 protein and at its C-terminus to the C-terminal portion of a Saccharomyces cerevisiae SED1 protein. In a further embodiment, the heterologous protein amino acid sequence is a minisatellite-like repeat sequence from a yeast cell wall protein. In particular embodiments, the yeast cell wall protein is selected from FLO1, FLO2, and FLO11. In exemplary embodiments, the chimeric surface anchor polypeptide has an amino acid sequence selected from SEQ ID NO: 34 or SEQ ID NO: 35.

In particular embodiments of the method, the bait comprises an amino acid sequence set forth in SEQ ID NO: 28, SEQ ID NO: 29, SEQ ID NO: 30, SEQ ID NO: 38, or SEQ ID NO: 41.

In particular embodiments, the one or more polynucleotides encoding the VL and VH are each operably linked to a second regulatable promoter. In a further embodiment, the second regulatable promoter is a GAL1 promoter.

In further embodiments of the method, the isolated yeast is Saccharomyces cerevisiae, which in particular embodiments, the isolated yeast host cell is diploid.

In further embodiments of the method, the VL and VH are human or humanized.

In further embodiments of the method, said plurality of polynucleotides encoding a VL represents a diverse population of VLs; and/or, wherein said plurality of polynucleotides encoding a VH represents a diverse population of VHs.

The present invention provides a diploid yeast host cell comprising (a) a polynucleotide encoding a bait comprising an immunoglobulin Fc domain fused to a cell surface anchor polypeptide, which said cell surface anchor polypeptide comprises more than 320 amino acids, operably linked to a regulatable promotor; (b) a polynucleotide encoding an immunoglobulin light chain variable domain (VL); and (c) a polynucleotide encoding an immunoglobulin heavy chain variable domain (VH).

In a further embodiment of the diploid yeast host cell, the host cell expresses (i) a non-tethered full-length bivalent antibody tetramer comprising two immunoglobulin heavy chains (HC), each HC comprising said VH, and two immunoglobulin light chains (LC), each LC comprising said VL: and/or (ii) a monovalent antibody fragment comprising one HC and one LC complexed with the Fc moiety of the bait.

In a further embodiment of the diploid yeast host cell, the VL is fused to an immunoglobulin light chain constant domain and the VH is fused to an immunoglobulin heavy chain constant domain having an Fc domain or immunoglobulin heavy chain CH1 domain and lacking an Fc domain.

In a further embodiment of the diploid yeast host cell, the immunoglobulin heavy chain constant domain is an IgG1, IgG2, IgG3, or IgG4 immunoglobulin constant domain.

In a further embodiment of the diploid yeast host cell, the Fc immunoglobulin domain is an IgG1, IgG2, IgG3 or IgG4 Fc immunoglobulin domain. In particular embodiments, the heavy chain Fc immunoglobulin domain comprises an N297A amino acid substitution, wherein the numbering is in accordance with the Eu numbering scheme. In a further embodiment of the diploid yeast host cell, the cell surface anchor polypeptide comprises between 400 to 700 amino acids.

In a further embodiment of the diploid yeast host cell, the regulatable promoter is a TetO7 promoter.

In a further embodiment of the diploid yeast host cell, the cell surface anchor polypeptide comprises a Saccharomyces cerevisiae SED1 protein. In particular embodiments, the cell surface anchor polypeptide comprises a Saccharomyces cerevisiae SED1 protein comprising about 401, 430, or 481 amino acids. In exemplary embodiments, the cell surface anchor polypeptide comprises a Saccharomyces cerevisiae SED1 protein comprising the amino acid sequence set forth in SEQ ID NO: 18, SEQ ID NO: 19, or SEQ ID NO: 20.

In a further embodiment of the diploid yeast host cell, the cell surface anchor polypeptide is a chimeric surface anchor polypeptide comprising a Saccharomyces cerevisiae SED1 protein and a heterologous protein.

In a further embodiment of the diploid yeast host cell, the cell surface anchor polypeptide is a chimeric surface anchor polypeptide comprising a heterologous protein amino acid sequence linked at its N-terminus to the N-terminal portion of a Saccharomyces cerevisiae SED1 protein and at its C-terminus to the C-terminal portion of a Saccharomyces cerevisiae SED1 protein. In particular embodiments, the heterologous protein amino acid sequence is a minisatellite-like repeat sequence from a yeast cell wall protein. In further embodiments, the yeast cell wall protein is selected from FLO1, FLO2, and FLO11. In exemplary embodiments, the chimeric surface anchor polypeptide has an amino acid sequence selected from SEQ ID NO: 34 or SEQ ID NO: 35.

In a further embodiment of the diploid yeast host cell, the bait comprises an amino acid sequence set forth in SEQ ID NO: 28, SEQ ID NO; 29, SEQ ID NO: 30, SEQ ID NO: 38, or SEQ ID NO: 41.

In a further embodiment of the diploid yeast host cell, the one or more polynucleotides encoding the VL and VH are each operably linked to a second regulatable promoter.

In a further embodiment of the diploid yeast host cell, the second regulatable promoter is a GAL1 promoter.

In a further embodiment of the diploid yeast host cell, the yeast is Saccharomyces cerevisiae.

In a further embodiment of the diploid yeast host cell, the immunoglobulin heavy chain and light chain variable domains are human or humanized.

The present invention provides a method for producing an antibody that binds specifically to a molecule of interest comprising (a) providing a library of yeast host cells, each yeast host cell comprising (i) a first polynucleotide encoding a bait comprising a heavy chain Fc immunoglobulin domain fused to a surface anchor polypeptide, which said cell surface anchor polypeptide comprises more than 320 amino acids, operably linked to a regulatable promotor: (ii) a second polynucleotide encoding an immunoglobulin light chain (LC) having a variable domain (VL); and (iii) a third polynucleotide encoding an immunoglobulin heavy chain (HC) having a variable domain (VH), wherein the second and third polynucleotides are each operably linked to a repressible second regulatable promoter: (b) cultivating the library of host cells in a first medium without inducing expression of the bait, the LC, and the HC for a time sufficient to produce a first culture of the library of host cells; (c) cultivating the first culture of the library of host cells in a medium comprising an inducer of the first regulatable promoter to induce expression of the bait, a derepresser to derepress the second regulatable promoter, and an inducer of the second regulatable promoter to induce expression of the heavy and light chains to provide an expression culture wherein the Fc of the bait displayed on the host cell surface is complexed with the heavy chain constant domain of a monovalent antibody fragment comprising one HC and one LC (H+L): (e) contacting the expression culture of the library with the molecule of interest conjugated to a detectable moiety to identify those yeast host cells in the library that display on the host cell surface a monovalent antibody fragment comprising one HC and one LC (H+L) that specifically bind the molecule of interest; and (f) isolating a yeast host cell from those yeast host cells in the library that display on the host cell surface a monovalent antibody fragment comprising one HC and one LC (H+L) that specifically bind the molecule of interest and cultivating the isolated yeast host cell under conditions that induce expression of the HC and LC and does not induce expression of the Fc bait, wherein the host cell secretes the antibody that binds specifically the molecule of interest.

In a further embodiment of the method, the VL is fused to an immunoglobulin light chain constant domain and the VH is fused to an immunoglobulin heavy chain constant domain having an Fc domain or immunoglobulin heavy chain CH1 domain and lacking an Fc domain.

In a further embodiment of the method, the immunoglobulin heavy chain constant domain is an IgG1, IgG2, IgG3, or IgG4 immunoglobulin constant domain or the Fc immunoglobulin domain is an IgG1, IgG2, IgG3 or IgG4 Fc immunoglobulin domain. In particular embodiments, the Fc domain comprises an N297A amino acid substitution, wherein the numbering is in accordance with the Eu numbering scheme.

In a further embodiment of the method, the surface anchor polypeptide comprises between 400 to 700 amino acids.

In a further embodiment of the method, the regulatable promoter is a TetO7 promoter.

In a further embodiment of the method, the surface anchor polypeptide comprises a Saccharomyces cerevisiae SED1 protein. In particular embodiments, the surface anchor polypeptide comprises a Saccharomyces cerevisiae SED1 protein comprising about 401, 430, or 481 amino acids. In further still embodiments, the cell surface anchor polypeptide comprises a Saccharomyces cerevisiae SED1 protein comprising the amino acid sequence set forth in SEQ ID NO: 18, SEQ ID NO: 19, or SEQ ID NO: 20.

In further embodiments of the method, the cell surface anchor polypeptide is a chimeric surface anchor polypeptide comprising a Saccharomyces cerevisiae SED1 protein and a heterologous protein. In a further embodiment, the cell surface anchor polypeptide is a chimeric surface anchor polypeptide comprising a heterologous protein amino acid sequence linked at its N-terminus to the N-terminal portion of a Saccharomyces cerevisiae SED1 protein and at its C-terminus to the C-terminal portion of a Saccharomyces cerevisiae SED1 protein. In a further embodiment, the heterologous protein amino acid sequence is a minisatellite-like repeat sequence from a yeast cell wall protein. In particular embodiments, the yeast cell wall protein is selected from FLO1, FLO2, and FLO11. In exemplary embodiments, the chimeric surface anchor polypeptide has an amino acid sequence selected from SEQ ID NO: 34 or SEQ ID NO: 35.

In particular embodiments of the method, the bait comprises an amino acid sequence set forth in SEQ ID NO: 28, SEQ ID NO: 29, SEQ ID NO: 30, SEQ ID NO: 38, or SEQ ID NO: 41.

In particular embodiments, the one or more polynucleotides encoding the VL and VH are each operably linked to a second regulatable promoter. In a further embodiment, the second regulatable promoter is a GAL1 promoter.

In further embodiments of the method, the isolated yeast is Saccharomyces cerevisiae, which in particular embodiments, the isolated yeast host cell is diploid.

In further embodiments of the method, the VL and VH are human or humanized.

In further embodiments of the method, said plurality of polynucleotides encoding a VL represents a diverse population of VLs: and/or, wherein said plurality of polynucleotides encoding a VH represents a diverse population of VHs.

### BRIEF DESCRIPTION OF THE DRAWINGS

FIG. 1A shows the surface display and co-secretion system of the present invention. Polynucleotides encoding an antibody and bait anchor are co-expressed in Saccharomyces cerevisiae. The S. cerevisiae cell expresses the bait on the cell surface, some of such are bound by a monovalent antibody fragment (comprising one heavy and one light chain) of the antibody that is also expressed. Some expressed antibody escapes bait binding and is, thus, soluble. Expression of the antibody on the cell surface can be confirmed by FACS analysis and a titer of the cellular antibody expression level can also be determined.

FIG. 1B shows the design and construction of fully human, heavy chain and light chain, antibody yeast display mating library. The HC (heavy chain) libraries consist of 10 selected human VH frameworks, whereas the HCDR1 and HCDR2 (CDR, complementarity-determining regions) regions of the variable domains are diversified using degenerated oligonucleotides to mimic the human germline antibody repertoires. A synthetic HCDR3 library of 6-18 residues is synthesized using TRIM (trinucleotide-directed mutagenesis) technology for the assembly of highly diversified HC CDR3 regions. The VH gene and HCDR3 regions are stitched together by PCR assembly. The variable region is further stitched together with the IgG constant region with N297A point mutation to form the human heavy chain library. The N297A mutation removes the N-glycosylation site at the CH2 region. The PCR assembled heavy chain library was transformed to a haploid S. cerevisiae mating type α yeast strain. The light chain (LC) libraries consist of nine selected human V kappa genes, whereas all three LC CDR regions of the variable domain were diversified using degenerated oligonucleotides to mimic the human germline antibody repertoires. The VL gene is stitched together with the human kappa constant region by PCR to form the human light chain library. The light chain library is transformed to a haploid S. cerevisiae mating type α yeast strain. The haploid yeast HC and LC libraries are mated together to form combinatorial, high diversity human antibody diploid libraries.

FIG. 2 shows an amino acid sequence comparison of the most commonly known ScSed1 (referred as SED1.320) with the newly identified long Sed1 variants (SED1.481) described herein. SED1.320 gene is curated in the Saccharomyces Genome Database. The amino acid sequence of ScSED1 (or SED1.320; SEQ ID NO: 13) is curated in SGD:S000002484, UniProtKB/Swiss-Prot: Q01589.1, SED1.320 sequence is obtained from the laboratory S. cerevisiae strain ATCC 204508/S288C. The SED1.320 gene encodes 338 amino acids in which position 1-18 is the signal peptide. The mature SED1.320 protein has 320 amino acid long. The amino acid sequence of SED1.481 is obtained from GenBank: AJV18036.1 (SEQ ID NO: 16). It was found by deep sequencing the wild-type yeast strain YJM1549. The mature SED1.481 protein is 481 amino acids long (excluding the signal peptide).

FIG. 3 shows the genealogy of yeast display and co-secretion human antibody mating beginning from parental S. cerevisiae strains BJ5464 and BJ5465.

FIG. 4 shows the genetic implementation of the described antibody display and co-secretion system. Three main components central to the described antibody display and co-secretion system are: (I) antibody heavy chain, (II) antibody light chain, and (III) surface anchor bait expression. The bait expression can be under the control of a different inducible promoter than the heavy and light chain expression. The bait expression can thus be turned on and off independent with antibody expression. When the bait expression is turned off, the resulting cell expresses the polynucleotide encoding the antibody heavy and light chains and produces full soluble antibody to the supernatant. Cellular expression levels of the antibody can then be confirmed, and a determination of the antibody affinity can also be performed.

FIG. 5 shows general strain construction procedure for testing different yeast Fc-bait surface display systems and confirming their efficiency, beginning from parental S. cerevisiae strain BJ5464 and BJ5465.

FIG. 6 shows a map of plasmid pGLY16289. A nucleic acid molecule encoding a human Fc N297A mutein fused to the S. cerevisiae SED1.320 protein surface anchor (320 amino acid long) is operably linked to a doxycycline inducible TetO7 promoter.

FIG. 7 shows a map of plasmid pGLY15562. A nucleic acid molecule encoding the anti-HER2 heavy chain is operably linked to the S. cerevisiae galactose inducible GAL1 Promoter. The anti-HER2 heavy chain comprises the trastuzumab heavy chain variable region fused to the human IgG1 constant region with an N297A mutation.

FIG. 8 shows a map of plasmid pGLY16304. A nucleic acid molecule encoding the anti-HER2 light chain is operably linked to the S. cerevisiae galactose inducible GAL1 Promoter. The anti-HER2 light chain comprises the trastuzumab light chain variable region fused to the human kappa constant region.

FIG. 9 shows doxycycline controllable Fc-SED1.320 anchor displayed on cell surface. Doxycycline concentrations ranging from 1 μg/mL to 10 μg/mL were used to induce synthetic TetO7 promoter for Fc-SED1 displayed on cell surface. Human Fc signal was detected using APC conjugated goat polyclonal F(ab′)₂ anti-human IgG Fcγ fragment specific detection reagent.

FIG. 10 shows flow cytometric analysis of Fc-SED1.320 bait mediated anti-HER2 displaying yeast binding to biotinylated human HER2 ectodomain antigen. The cells were dually labeled with goat anti-human kappa Alexa 647 (Y-axis), and biotinylated human HER2 ectodomain protein, and then reacted with streptavidin-R-phycoerythrin (R-PE) conjugated (X-axis). Cells were grown in galactose induction media (left panel) or dextrose repressing media (right-panel).

FIG. 11 shows a map of plasmid pGLY16315. A nucleic acid molecule encoding the S. cerevisiae AGA1 protein is operably linked to the galactose inducible GAL1 promoter. The AGA1 nucleic acid molecule encodes the anchorage subunit of α-agglutinin of a mating type yeast cell.

FIG. 12 shows a map of plasmid pGLY16327. A nucleic acid molecule encoding the human IgG1 Fc N297A mutein fused to the N-terminus of S. cerevisiae AGA2 protein to make the Fc-AGA2 protein operably linked to a doxycycline inducible TetO7 promoter.

FIG. 13 shows flow cytometric analysis of AGA1/AGA2 Fc bait mediated anti-HER2 displaying yeast binding to biotinylated human HER2 Ectodomain antigen. The cells were dually labeled with goat anti-human kappa Alexa 647 (Y-axis) and biotinylated human HER2 ectodomain protein, and then reacted with streptavidin-R-phycoerythrin (R-PE) conjugated (X-axis). Cells were grown in galactose induction media.

FIG. 14A and FIG. 14B show amino acid sequence alignment of the most commonly known ScSed1 (referred as SED1.320) with the naturally occurring longer Sed1 variants isolated from wild-type S. cerevisiae strains. The SED1.320 amino acid sequence was obtained from S. cerevisiae strain ATCC 204508/S288C (SEQ ID NO: 13). The SED1.320 gene encodes 338 amino acid in which amino acids 1-18 comprise a signal peptide. The mature SED1.320 protein is 320 amino acids long. Sequence of SED1.401 was obtained from GenBank: AF510225.1 (SEQ ID NO: 14). The matured SED1.401 protein is 401 amino acids long (excluding the first 18 amino acid signal peptide). Sequence of SED1.430 was obtained from GenBank: AJU57922 (SEQ ID NO: 15). It was found by deep sequencing wild-type yeast strain YJM189. The mature SED1.430 protein is 430 amino acid long (excluding the signal peptide). The amino acid sequence of SED1.481 was obtained from GenBank: AJV18036.1 (SEQ ID NO: 16). It was found by deep sequencing wild-type yeast strain YJM1549. The mature SED1.481 protein has 481 amino acid long (excluding the signal peptide).

FIG. 15 shows a map of plasmid pGLY16356. A nucleic acid molecule encoding the human Fc N297A mutein fused to the S. cerevisiae long SED1.481 protein anchor (481 amino acid) is operably linked to a doxycycline inducible TetO7 promoter.

FIG. 16 shows a map of plasmid pGLY16309. A nucleic acid molecule encoding the anti-TNFα heavy chain expression is operably linked to the S. cerevisiae galactose inducible GAL1 Promoter. The anti-TNFα heavy chain comprises the adalimumab heavy chain variable region fused to the human IgG1 constant region having an N297A mutation.

FIG. 17 shows a map of plasmid pGLY16304. A nucleic acid molecule encoding the anti-TNFα light chain operably linked to the S. cerevisiae galactose inducible GAL1 Promoter. The anti-TNFα light chain comprises the adalimumab light chain variable region fused to the human kappa constant region.

FIG. 18A shows a flow cytometric analysis of Fc-SED1.481 bait mediated anti-HER2 monovalent antibody fragment (H+L) display. The cells were dually labeled with goat anti-human LC kappa Alexa 647 (Y-axis) and biotinylated human HER2 ectodomain protein, and then reacted with streptavidin-R-Phycoerythrin (R-PE) conjugated (X-axis).

FIG. 18B shows a flow cytometric analysis of Fc-SED1.481 bait mediated anti-TNFα antibody display. The cells were dually labeled with goat anti-human LC kappa Alexa 647 (Y-axis) and biotinylated human TNFα homotrimer, and then reacted with streptavidin-R-phycoerythrin (R-PE) conjugated (X-axis).

FIG. 19 shows the amino acid sequence composition of the artificial SED1-FLO5-680aa fusion protein anchor (SEQ ID NO:34). The SED1-FLO5-680aa protein anchor is 680 amino acid long and is comprised of, in the order of N-terminus to C-terminus: (1) N-terminal SED1.320 region (SED1.320 amino acid 1-119), (2) yeast FLO5 protein mid-region (FLO5 amino acid 278-637), and (3) C-terminal SED1.320 region (SED1.320 amino acid 120-320). The mid-region of FLO5 protein encodes 8× approximate 45 amino acid threonine-rich tandem repeats. The C-terminal SED1.320 portion contains the GPI anchor.

FIG. 20 shows the amino acid sequence composition of the artificial SED1-FLO1-660aa fusion protein anchor (SEQ ID NO: 35). The SED1-FLO1-660aa protein anchor is 660 amino acid long and is comprised of, in the order of N-terminus to C-terminus: (1) N-terminal SED1.320 region (SED1.320 amino acid 1-119), (2) yeast FLO1 protein mid-region (FLO1 amino acid 818-1167), and (3) C-terminal SED1.320 region (SED1.320 amino acid 120-320). The mid-region of FLO1 protein encodes some threonine and serine-rich tandem repeat sequences. The C-terminal SED1.320 portion contains the GPI anchor.

FIG. 21 shows a map of plasmid pGLY16350. A nucleic acid molecule encoding the human Fc N297A mutein fused to the artificial S. cerevisiae SED1-FLO5-680 (680 aa) protein anchor operably linked to the doxycycline inducible TetO7 promoter.

FIG. 22 shows a map of plasmid pGLY16348. A nucleic acid molecule encoding the human Fc N297A mutein fused to the artificial S. cerevisiae SED1-FLO1-660 (660 aa) protein anchor operably linked to the doxycycline inducible TetO7 promoter.

FIG. 23A shows flow cytometric analysis of Fc-SED1-FLO5-680 bait mediated anti-HER2 monovalent antibody fragment (H+L) display. The cells were dually labeled with goat anti-human kappa Alexa 647 (Y-axis) and biotinylated human HER2 ectodomain protein, and then reacted with streptavidin-R-phycoerythrin (R-PE) conjugated (X-axis). Cells were grown in galactose induction media.

FIG. 23B shows flow cytometric analysis of Fc-SED1-FLO1-660 bait mediated anti-HER2 monovalent antibody fragment (H+L) display. The cells were dually labeled with goat anti-human kappa Alexa 647 (Y-axis) and biotinylated human HER2 ectodomain protein, and then reacted with streptavidin-R-phycoerythrin (R-PE) conjugated (X-axis). Cells were grown in galactose induction media.

FIG. 24 shows a linear DNA fragment construct that targets S. cerevisiae TRP1 locus and contains in tandem 11 nucleic acid regions encoding (1) TRP1-5′ region, (2) Lox66, a mutant LoxP, (3) A. gossypii TEF promoter, (4) A G418 resistant aphA1 gene, (5) A. gossypii TEF terminator, (6) reversed GAL1 promotor, (7) GAL10 promoter, (8), a Cre-recombinase expression cassette encoded by the Cre ORF of P1 Bacteriophage, (9) P. pastoris AOX1 transcription terminator sequences, (10) Lox71, a mutant LoxP, and (11) TRP1-3′ region.

FIG. 25 shows a map of plasmid pGLY16336. A nucleic acid molecule encoding the S. cerevisiae PDI1 is operably linked to the constitutive GAPDH promoter. PDI1 encodes yeast protein disulfide isomerase.

FIG. 26 shows a map of plasmid pLIB-HC. Plasmid pLIB-HC is used to build the GAL1-promoter driven antibody heavy chain library. The heavy chain library comprises a nucleic acid molecule encoding the S. cerevisiae alpha mating factor pre-signal peptide and a nucleic acid molecule encoding the human IgG1 constant region with N297A mutation flanking a region for inserting by homologous recombination nucleic acid molecules of a variable heavy chain region (VH) library synthesized as separate linear nucleic acid molecules to produce a plurality of plasmids, each plasmid containing a particular nucleic acid molecule from the V_{H} library. The plasmid also contains the pUC19 DNA sequence for maintenance in E. coli, and a DNA sequence encoding the S. cerevisiae LEU2 open reading frame as a selection marker.

FIG. 27 shows a map of plasmid pLIB-LC. Plasmid pLIB-LC is used to build the GAL1-promoter driven antibody light chain library. The light chain library comprises a nucleic acid molecule encoding the S. cerevisiae alpha mating factor pre signal peptide and a nucleic acid molecule encoding the human kappa constant region flanking a region for inserting by homologous recombination nucleic acid molecules of a variable light chain region (V L) library synthesized as separate linear nucleic acid molecules to produce a plurality of plasmids, each plasmid containing a particular nucleic acid molecule from the VL library. The plasmid also contains the pUC19 DNA sequence for maintenance in E. coli, and a DNA sequence encoding the S. cerevisiae TRP1 open reading frame as a selection marker.

FIG. 28 shows a table of nine LC libraries that were individually transformed into yeast strain BDB360 (MATα). The size of every LC library is greater than 10⁹.

FIG. 29 shows a table of 20 HC libraries that were individually transformed into yeast strain BDB535 (MATa). The size of every HC library is greater than 10⁹.

FIG. 30 shows two HC libraries from the same V gene(s) with different HCDR3 length distribution (6-10 and 11-18 amino acids) that were pooled together in a 1:1 ratio to create a combined V gene heavy chain library.

FIG. 31 shows Construction of a synthetic fully human yeast display mating library (see legend of FIG. 1B for details).

FIG. 32 shows a table of the media used for growth, de-repression, and induction of yeast display library.

FIG. 33A-1 and FIG. 33-A2 show flow cytometric expressional analysis of V_{H}1-18 heavy chain library pairing with nine individual V_{L} libraries. The cells were dually labeled with goat anti-human LC kappa Alexa 647 (Y-axis) and biotinylated llama V_{HH} anti-human C_{H}1, then and reacted with NeutrAvidin-R-phycoerythrin (R-PE) conjugated (X-axis). Cells were grown in galactose induction media.

FIG. 33B shows flow cytometric expressional analysis of V_{H}1-46 heavy chain library pairing with nine individual V_{L} libraries. The cells were dually labeled with goat anti-human LC kappa Alexa 647 (Y-axis) and biotinylated llama V_{HH} anti-human C_{H}1, and then reacted with NeutrAvidin-R-phycoerythrin (R-PE) conjugated (X-axis). Cell were grown in galactose induction media.

FIG. 33C shows flow cytometric expressional analysis of V_{H}1-69 heavy chain library pairing with nine individual V_{L} libraries. The cells were dually labeled with goat anti-human LC kappa Alexa 647 (Y-axis) and biotinylated llama V_{HH} anti-human C_{H}1, then and reacted with NeutrAvidin-R-phycoerythrin (R-PE) conjugated (X-axis). Cell were grown in galactose induction media.

FIG. 33D shows flow cytometric expressional analysis of V_{H}3-7 heavy chain library pairing with nine individual V_{L} libraries. The cells were dually labeled with goat anti-human LC kappa Alexa 647 (Y-axis) and biotinylated llama V_{HH} anti-human C_{H}1, then and reacted with NeutrAvidin-R-phycoerythrin (R-PE) conjugated (X-axis). Cell were grown in galactose induction media.

FIG. 33E shows flow cytometric expressional analysis of V_{H}3-23 heavy chain library pairing with nine individual V_{L} libraries. The cells were dually labeled with goat anti-human LC kappa Alexa 647 (Y-axis) and biotinylated llama V_{HH} anti-human C_{H}1, then and reacted with NeutrAvidin-R-phycoerythrin (R-PE) conjugated (X-axis). Cells were grown in galactose induction media.

FIG. 33F shows flow cytometric expressional analysis of V_{H}3-66 heavy chain library pairing with nine individual V_{L} libraries. The cells were dually labeled with goat anti-human LC kappa Alexa 647 (Y-axis) and biotinylated llama V_{HH} anti-human C_{H}1, then and reacted with NeutrAvidin-R-phycoerythrin (R-PE) conjugated (X-axis). Cells were grown in galactose induction media.

FIG. 33G shows flow cytometric expressional analysis of V_{H}3-72 heavy chain library pairing with nine individual V_{L} libraries. The cells were dually labeled with goat anti-human LC kappa Alexa 647 (Y-axis) and biotinylated llama V_{HH} anti-human C_{H}1, then and reacted with NeutrAvidin-R-phycoerythrin (R-PE) conjugated (X-axis). Cells were grown in galactose induction media.

FIG. 33H shows flow cytometric expressional analysis of V_{H}4-4 heavy chain library pairing with nine individual V_{L} libraries. The cells were dually labeled with goat anti-human LC kappa Alexa 647 (Y-axis) and biotinylated llama V_{HH} anti-human C_{H}1, then and reacted with NeutrAvidin-R-phycoerythrin (R-PE) conjugated (X-axis). Cells were grown in galactose induction media.

FIG. 33I shows flow cytometric expressional analysis of V_{H}4-59 heavy chain library pairing with nine individual V_{L} libraries. The cells were dually labeled with goat anti-human LC kappa Alexa 647 (Y-axis) and biotinylated llama V_{HH} anti-human C_{H}1, then and reacted with NeutrAvidin-R-phycoerythrin (R-PE) conjugated (X-axis). Cells were grown in galactose induction media FIG. 33J shows flow cytometric expressional analysis of V_{H}5-51 heavy chain library pairing with nine individual V_{L} libraries. The cells were dually labeled with goat anti-human LC kappa Alexa 647 (Y-axis) and biotinylated llama V_{HH} anti-human C_{H}1, then and reacted with NeutrAvidin-R-phycoerythrin (R-PE) conjugated (X-axis). Cells were grown in galactose induction media FIG. 34 shows a table of the composition of final six mixed V_{H}×V_{L} libraries.

FIGS. 35A and 35B shows flow cytometric analysis of pooled V_{H}×V_{L} antibody library pre-sorted and after-sorted using Kappa and C_{H}1 expression profiles. FIG. 35A is a cartoon showing where the anti-CH1-PE and anti-kappa 647 antibodies bind to a captured antibody. FIG. 35B left panel shows data relating to unsorted pooled V_{H}×V_{L} library, and the right panel shows data relating to cells that were sorted using goat anti-human kappa Alexa 647 (Y-axis) and biotinylated llama V_{H}H anti-human CH1, and then reacted with NeutrAvidin-R-Phycoerythrin (R-PE) conjugated (X-axis). P1 quadrilateral indicates the sorting gate.

FIG. 36A shows octet measuring of the antibody secretion titer from the randomly selected yeast clones sorted in FIG. 35B.

FIG. 36B shows purified antibodies generated by the strains in A1, B1, C1, D1 run on non-reducing SDS-PAGE to confirm quality and assembly.

### DETAILED DESCRIPTION OF THE INVENTION

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

The term “Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (KD). Affinity can be measured by common methods known in the art, including kinetic exclusion assay also known by the registered trademark KinExA and surface plasmon resonance (SPR) also known by the registered trademark Biacore. Specific illustrative and exemplary embodiments for measuring binding affinity are described in the following.

The term “administration” and “treatment,” as it applies to an animal, human, experimental subject, cell, tissue, organ, or biological fluid, refers to contact of an exogenous pharmaceutical, therapeutic, diagnostic agent, or composition comprising an antibody to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell. The term “subject” includes any organism, preferably an animal, more preferably a mammal (e.g., human, rat, mouse, dog, cat, rabbit). In a preferred embodiment, the term “subject” refers to a human.

The term “amino acid” refers to a simple organic compound containing both a carboxyl (—COOH) and an amino (—NH₂) group. Amino acids are the building blocks for proteins, polypeptides, and peptides. Amino acids occur in L-form and D-form, with the L-form in naturally occurring proteins, polypeptides, and peptides. Amino acids and their code names are set forth in the following chart.

| Amino acid | Three letter code | One letter code |
|---|---|---|
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

As used herein, the term “antibody” or “immunoglobulin” as used herein refers to a glycoprotein comprising either (a) at least two heavy chains (HCs) and two light chains (LCs) inter-connected by disulfide bonds, or (b) in the case of a species of camelid antibody, at least two heavy chains (HCs) inter-connected by disulfide bonds. Each HC is comprised of a heavy chain variable region or domain (VH) and a heavy chain constant region or domain. Each light chain is comprised of an LC variable region or domain (VL) and a LC constant domain. In certain naturally occurring IgG, IgD and IgA antibodies, the heavy chain constant region is comprised of three domains, CH1, CH2 and CH3. In general, the basic antibody structural unit for antibodies is a Y-shaped tetramer comprising two HC/LC pairs (2H+2L), except for the species of camelid antibodies comprising only two HCs (2H), in which case the structural unit is a homodimer. Each tetramer includes two identical pairs of polypeptide chains, each pair having one LC (about 25 kDa) and HC chain (about 50-70 kDa) (H+L). Each HC:LC pair comprises one VH: one VL pair that binds to the antigen. The VH: VL pair of the antibody, which comprises the CH1 domain of the HC and the light chain constant domain further may be referred to by the term “Fab”. Thus, each antibody tetramer comprises two Fabs, one per each arm of the Y-shaped antibody above the hinge region. When not associated with the Fc domain, the Fab is referred to as Fab fragment.

The LC constant domain is comprised of one domain, CL. The human VH includes seven family members: VH1, VH2, VH3, VH4, VH5, VH6, and VH7; and the human VL includes 16 family members: Vκ1, Vκ2, Vκ3, Vκ4, Vκ5, Vκ6, Vλ1, Vλ2, Vλ3, Vλ4, Vλ5, Vλ6, Vλ7, Vλ8, Vλ9, and Vλ10. Each of these family members can be further divided into particular subtypes. The VH and VL can be further subdivided into regions of hypervariability, termed complementarity determining region (CDR) areas, interspersed with regions that are more conserved, termed framework regions (FR). Each VH and VL is composed of three CDR regions and four FR regions, arranged from amino-terminus to carboxy-terminus in the following order: FR1, CDR1, FR2, CDR2, FR3, CDR3, FR4. Numbering of the amino acids in a VH or VHH may be determined using the Kabat numbering scheme. See Béranger, et al., Ed. Ginetoux. Correspondence between the IMGT unique numbering for C-DOMAIN, the IMGT exon numbering, the Eu and Kabat numberings: Human IGHG. Created: 17/05/2001. Version: 08/06/2016, which is accessible at www.imgt.org/IMGTScientificChart/Numbering/Hu_IGHGnber.html).

The constant regions of the antibodies may mediate the binding of the immunoglobulin to host tissues or factors, including various cells of the immune system (e.g., effector cells) and the first component (C1q) of the classical complement system. Typically, the numbering of the amino acids in the heavy chain constant domain begins with number 118, which is in accordance with the Eu numbering scheme. The Eu numbering scheme is based upon the amino acid sequence of human IgG1 (Eu), which has a constant domain that begins at amino acid position 118 of the amino acid sequence of the IgG1 described in Edelman et al., Proc. Natl. Acad. Sci. USA. 63: 78-85 (1969), and is shown for the IgG1, IgG2, IgG3, and IgG4 constant domains in Béranger, et al., op. cit.

The variable regions of the heavy and light chains contain a binding domain comprising the CDRs that interacts with an antigen. A number of methods are available in the art for defining CDR sequences of antibody variable domains (see Dondelinger et al., Frontiers in Immunol. 9: Article 2278 (2018)). The common numbering schemes include the following.
Kabat numbering scheme is based on sequence variability and is the most commonly used (See Kabat et al. Sequences of Proteins of Immunological Interest, 5th Ed. Public Health Service, National Institutes of Health, Bethesda, Md. (1991) (defining the CDR regions of an antibody by sequence);
Chothia numbering scheme is based on the location of the structural loop region (See Chothia & Lesk J. Mol. Biol. 196: 901-917 (1987): Al-Lazikani et al., J. Mol. Biol. 273: 927-948 (1997));
AbM numbering scheme is a compromise between the two used by Oxford Molecular's AbM antibody modelling software (see Karu et al, ILAR Journal 37, 132-141 (1995),
Contact numbering scheme is based on an analysis of the available complex crystal structures (See www.bioinf.org.uk: Prof. Andrew C. R Martin's Group; Abhinandan & Martin, Mol. Immunol. 45:3832-3839 (2008).
IMGT (ImMunoGeneTics) numbering scheme is a standardized numbering system for all the protein sequences of the immunoglobulin superfamily, including variable domains from antibody light and heavy chains as well as T cell receptor chains from different species and counts residues continuously from 1 to 128 based on the germ-line V sequence alignment (see Giudicelli et al., Nucleic Acids Res. 25:206-11 (1997): Lefranc, Immunol Today 18:509(1997); Lefranc et al., Dev Comp Immunol. 27:55-77 (2003)).

The following general rules disclosed in www.bioinforg.uk: Prof Andrew C. R. Martin's Group and reproduced in Table 1 below may be used to define the CDRs in an antibody sequence that includes those amino acids that specifically interact with the amino acids comprising the epitope in the antigen to which the antibody binds. There are rare examples where these generally constant features do not occur; however, the Cys residues are the most conserved feature.

TABLE 1

| Loop | Kabat | AbM | Chothia¹ | Contact² | IMGT |
|---|---|---|---|---|---|
| L1 | L24-L34 | L24-L34 | L24-L34 | L30-L36 | L27-L32 |
| L2 | L50-L56 | L50-L56 | L50-L56 | L46-L55 | L50-L51 |
| L3 | L89-L97 | L89-L97 | L89-L97 | L89-L96 | L89-L97 |
| H1 | H31-H35B | H26-H35B | H26-H32 . . . 34 | H30-H35B | H26-H35B |
| | (Kabat Numbering)³ | | | | |
| H1 | H31-H35 | H26-H35 | H26-H32 | H30-H35 | H26-H33 |
| | (Chothia Numbering) | | | | |
| H2 | H50-H65 | H50-H58 | H52-H56 | H47-H58 | H51-H56 |
| H3 | H95-H102 | H95-H102 | H95-H102 | H93-H101 | H93-H102 |

¹Some of these numbering schemes (particularly for Chothia loops) vary depending on the individual publication examined.
²Any of the numbering schemes can be used for these CDR definitions, except the Contact numbering scheme uses the Chothia or Martin (Enhanced Chothia) definition.
³The end of the Chothia CDR-H1 loop when numbered using the Kabat numbering convention varies between H32 and H34 depending on the length of the loop. (This is because the Kabat numbering scheme places the insertions at H35A and H35B.)
If neither H35A nor H35B is present, the loop ends at H32
If only H35A is present, the loop ends at H33
If both H35A and H35B are present, the loop ends at H34

The entire nucleotide sequence of the heavy chain and light chain variable regions are commonly numbered according to Kabat while the three CDRs within the variable region may be defined according to any one of the aforementioned numbering schemes.

In general, the state of the art recognizes that in many cases, the CDR3 region of the heavy chain is the primary determinant of antibody specificity, and examples of specific antibody generation based on CDR3 of the heavy chain alone are known in the art (e.g., Beiboer et al., J. Mol. Biol. 296: 833-849 (2000); Klimka et al., British J. Cancer 83: 252-260 (2000). Rader et al., Proc. Natl. Acad. Sci. USA 95: 8910-8915 (1998); Xu et al., Immunity 13: 37-45 (2000).

As used herein, the term “monovalent antibody fragment” comprises one half of an antibody, i.e., the antibody heavy chain (VH-CH1-CH2-CH3) bound to the antibody light chain (VL-CL) comprising three paired CDRs, e.g., wherein CH1 and CL are bound by a disulfide bridge, which monovalent antibody fragment is capable of detectably binding an antigen.

As used herein, the term “divalent antibody fragment” comprises both monovalent antibody fragments bound by disulfide bridges between the heavy chain constant domains to form a 2H+2L tetramer.

As used herein, the term “Fc domain”, or “Fc” as used herein is the crystallizable fragment domain or region obtained from an antibody that comprises the CH2 and CH3 domains of an antibody. In an antibody, the two Fc domains are held together by two or more disulfide bonds and by hydrophobic interactions of the CH3 domains. The Fc domain may be obtained by digesting an antibody with the protease papain. Typically, amino acids in the Fc domain are numbered according to the Eu numbering convention (See Edelmann et al., Biochem. 63: 78-85 (1969)).

The term “antigen” as used herein refers to any foreign substance which induces an immune response in the body.

The terms “cell.” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny will have precisely identical DNA content, due to deliberate or inadvertent mutations. Mutant progeny that have the same function or biological activity as screened for in the originally transformed cell are included. Where distinct designations are intended, it will be clear from the context.

The term “control sequences” or “regulatory sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to use promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “expression” as used herein is defined as the transcription and/or translation of a particular nucleotide sequence.

The term “gene” is used broadly to refer to any segment of nucleic acid associated with a biological function. Thus, genes include coding sequences and/or the regulatory sequences required for their expression. For example, “gene” refers to a nucleic acid fragment that expresses mRNA, functional RNA, or specific protein, including regulatory sequences. “Genes” also include nonexpressed DNA segments that, for example, form recognition sequences for other proteins. “Genes” can be obtained from a variety of sources, including cloning from a source of interest or synthesizing from known or predicted sequence information, and may include sequences designed to have desired parameters.

The term “germline” or “germline sequence” refers to a sequence of unrearranged immunoglobulin DNA sequences. Any suitable source of unrearranged immunoglobulin sequences may be used. Human germline sequences may be obtained, for example, from JOINSOLVER® germline databases on the website for the National Institute of Arthritis and Musculoskeletal and Skin Diseases of the United States National Institutes of Health. Mouse germline sequences may be obtained, for example, as described in Giudicelli et al. (2005) Nucleic Acids Res. 33:D256-D261.

The term “library” as used herein is, typically, a collection of related but diverse polynucleotides that are, in general, in a common vector backbone. For example, a light chain or heavy chain immunoglobulin library may contain polynucleotides, in a common vector backbone, that encode light and/or heavy chain immunoglobulins, which are diverse but related in their nucleotide sequence; for example, which immunoglobulins are functionally diverse in their abilities to form complexes with other immunoglobulins, e.g., in an antibody display system of the present invention, and bind a particular antigen.

The terms “diverse population of VHs” and “diverse population of VLs” refers to a library of VH or VL wherein there are a large number of VH or VL variants therein. A diverse population of VH or VL will usually have a complexity of about 10⁶ to 10⁹ or more VH or VL variants therein. The library may be obtained from natural sources, for example, mouse, rat, rabbit, camelid, or the like, which have or have not been inoculated with an immunogen. Alternatively, the library may be a synthetic library based on computational in silico design and gene synthesis and the CDR design and composition is precisely defined and controlled. Semi-synthetic libraries comprise both CDRs from natural sources as well as in silico design of defined parts.

The term “polynucleotides” discussed herein form part of the present invention. A “polynucleotide”, “nucleic acid” or “nucleic acid molecule” include DNA and RNA, single- or double-stranded. Polynucleotides e.g., encoding an immunoglobulin chain or component of the antibody display system of the present invention (e.g., a bait), may, in an embodiment of the invention, be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like.

Polynucleotides e.g., encoding an immunoglobulin chain or component of the antibody display system of the present invention, may be operably associated with a promoter. A “promoter” or “promoter sequence” is, in an embodiment of the invention, a DNA regulatory region capable of binding an RNA polymerase in a cell (e.g., directly or through other promoter-bound proteins or substances) and initiating transcription of a coding sequence. A promoter sequence is, in general, bounded at its 3′ terminus by the transcription initiation site and extends upstream (5′ direction) to include the minimum number of bases or elements necessary to initiate transcription at any level. Within the promoter sequence may be found a transcription initiation site (conveniently defined, for example, by mapping with nuclease Si), as well as protein binding domains (consensus sequences) responsible for the binding of RNA polymerase. The promoter may be operably associated with other expression control sequences, including enhancer and repressor sequences or with a nucleic acid of the invention. Promoters which may be used to control gene expression include, but are not limited to, cytomegalovirus (CMV) promoter (U.S. Pat. Nos. 5,385,839 and 5,168,062), the SV40 early promoter region (Benoist, et al., (1981) Nature 290:304-310), the promoter contained in the 3′ long terminal repeat of Rous sarcoma virus (Yamamoto, et al., (1980) Cell 22:787-797), the herpes thymidine kinase promoter (Wagner, et al., (1981) Proc. Natl. Acad. Sci. USA 78:1441-1445), the regulatory sequences of the metallothionein gene (Brinster, et al., (1982) Nature 296:3942); prokaryotic expression vectors such as the β-lactamase promoter (Villa-Komaroff, et al., (1978) Proc. Natl. Acad. Sci. USA 75:3727-3731), or the tac promoter (DeBoer, et al., (1983) Proc. Natl. Acad. Sci. USA 80:21-25); see also “Useful proteins from recombinant bacteria” in Scientific American (1980) 242:74-94; and promoter elements from yeast or other fungi such as the Gal 4 promoter, the ADC (alcohol dehydrogenase) promoter, PGK (phosphoglycerol kinase) promoter or the alkaline phosphatase promoter.

The terms “vector”, “cloning vector” and “expression vector” include a vehicle (e.g., a plasmid) by which a DNA or RNA sequence can be introduced into a host cell, so as to transform the host and, optionally, promote expression and/or replication of the introduced sequence. Polynucleotides encoding an immunoglobulin chain or component of the antibody display system of the present invention (e.g., a bait) may, in an embodiment of the invention, be in a vector.

The terms “N-glycan” and “glycoform” are used interchangeably and refer to an N-linked oligosaccharide, e.g., one that is attached by an asparagine-N-acetylglucosamine linkage to an asparagine residue of a polypeptide. N-linked glycoproteins contain an N-acetylglucosamine residue linked to the amide nitrogen of an asparagine residue in the protein. Predominant sugars found on glycoproteins are glucose, galactose, mannose, fucose, N-acetylgalactosamine (GalNAc), N-acetylglucosamine (GlcNAc) and sialic acid (e.g., N-acetyl-neuraminic acid (NANA)).

N-glycans have a common pentasaccharide core of Man₃GlcNAc₂ (“Man” refers to mannose: “Glc” refers to glucose: and “NAc” refers to N-acetyl; GlcNAc refers to N-acetylglucosamine). N-glycans differ with respect to the number of branches (antennae) comprising peripheral sugars (e.g., GlcNAc, galactose, fucose and sialic acid) that are added to the Man3GlcNAc2 (“Man3”) core structure which is also referred to as the “triammnose core”, the “pentasaccharide core” or the “paucimannose core”. N-glycans are classified according to their branched constituents (e.g., high mannose, complex or hybrid). A “high mannose” type N-glycan has five or more mannose residues. A “complex” type N-glycan typically has at least one GlcNAc attached to the 1,3 mannose arm and at least one GlcNAc attached to the 1,6 mannose arm of a “trimannose” core. Complex N-glycans may also have galactose (“Gal”) or N-acetylgalactosamine (“GalNAc”) residues that are optionally modified with sialic acid or derivatives (e.g., “NANA” or “NeuAc”, where “Neu” refers to neuraminic acid and “Ac” refers to acetyl). Complex N-glycans may also have intrachain substitutions comprising “bisecting” GlcNAc and core fucose (“Fuc”). Complex N-glycans may also have multiple antennae on the “trimannose core,” often referred to as “multiple antennary glycans.” A “hybrid” N-glycan has at least one GlcNAc on the terminal of the 1.3 mannose arm of the trimannose core and zero or more mannoses on the 1,6 mannose arm of the trimannose core. The various N-glycans are also referred to as “glycoforms.” “PNGase”, or “glycanase” or “glucosidase” refer to peptide N-glycosidase F (EC 3.2.2.18).

The term “acceptable affinity” refers to antibody or antigen-binding fragment affinity for the antigen which is at least 10⁻³ M or a greater affinity (lower number), e.g., 10⁻³ M, 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M or 10⁻¹² M.

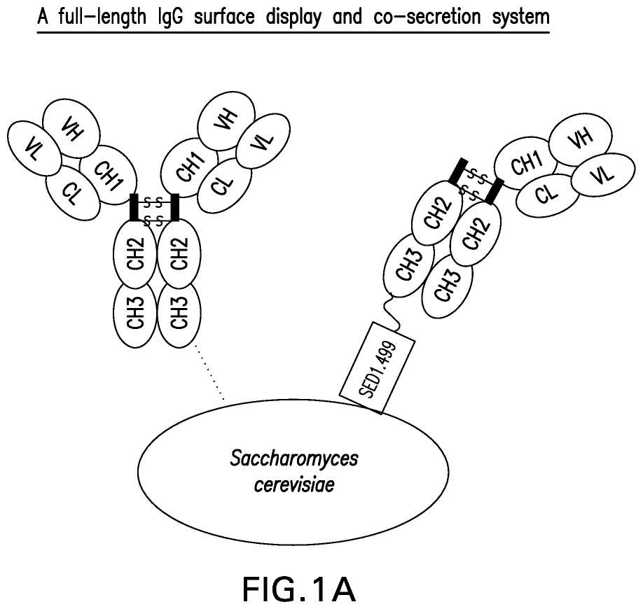

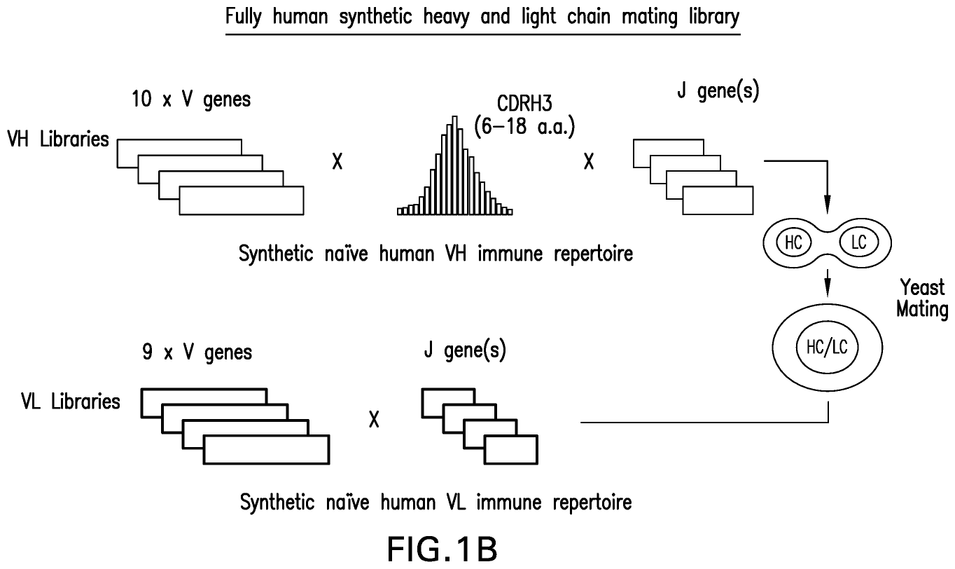

The term “tethered” refers to a monovalent antibody bound or associated to the Fc polypeptide comprising the bait anchored to the cell surface of a host cell expressing the monovalent antibody. For example. FIG. 1A on the right side shows a monovalent antibody expressed by the host cell tethered to a bait on the cell surface wherein the bait comprises an Fc domain fused to cell surface anchoring protein SED1.499.

The term “non-tethered” refers to a bivalent antibody tetramer that is not bound or associated to the Fc polypeptide comprising the bait anchored to the cell surface and which is instead secreted into the cell culture medium. For example, FIG. 1A on the left side shows a bivalent antibody tetramer being secreted from the host.

### Yeast Antibody Display System

The present invention provides a method for the display, secretion, and construction of very large size full-length IgG libraries in Saccharomyces cerevisiae for antibody discovery and selection. The method relies on the homo-dimerization of the Fc portion of one half of an IgG tetramer to a surface-anchored “bait” Fc, which results in tethering of functional “half” IgGs to the outer cell wall of S. cerevisiae for identifying host cells that express an IgG that binds an antigen of interest without interfering with secretion of full-length IgG tetramers from the same host cell.

U.S. Pat. No. 9,365,846 describes the display and secretion of full-length IgG in the yeast Pichia pastoris using a Saccharomyces cerevisiae SED1 cell surface anchor protein (ScSED1) for displaying antibody on the cell surface. However, the present invention provides a number of improvements or advantages over the display technology disclosed in the patent:

For example, when we tried to use the ScSED1 yeast surface membrane anchor protein, as described in the U.S. Pat. No. 9,365,846B2 to display antibodies in Pichia pastoris, to display full-length antibody in Saccharomyces cerevisiae, we found that ScSED1 does not display IgG1 molecules efficiently. We also found that the commonly used Saccharomyces AGA1-AGA2 display anchor is not compatible with the Fc-mediated IgG1 capture/display as described herein.

However, we discovered that by using a long, naturally occurring SED1 variant (called SED1.499) or long engineered semi-synthetic surface anchor proteins exemplified by SED1-FLO1-660, SED1-FLO1-678, and SED1-FLO5-680, functionally active “half” IgGs can be efficiently displayed on the cell wall of Saccharomyces cerevisiae via Fc mediated mechanism (See FIG. 1).

We further developed an efficient yeast transformation protocol that enables up to 10×10⁹ yeast transformant cells to be routinely achieved in one day, e.g., construction of greater than 10×10⁹ human IgG heavy chain yeast libraries and greater than 10×10⁹ light chain yeast libraries. By establishing a high efficiency large-scale yeast mating protocol, mating greater than 10×10⁹ heavy chain yeast library cells with greater than 10×10⁹ light chain yeast library cells allowed for very large combinational heavy×light antibody display libraries to be constructed.

The present invention provides an antibody display system, composition or kit comprising (1) a Saccharomyces cerevisiae host cell and (2) a bait comprising an Fc (e.g., a human Fc, e.g., an Fc comprising a CH2-CH3 polypeptide or CH2-CH3 polypeptide comprising a hinge region) fused, at the N- or C-terminus, (optionally, by a peptide linker such as GGG) to a surface anchor polypeptide having an amino acid sequence of greater than 320 amino acids, which bait is optionally linked to a signal sequence (e.g., an alpha mating factor signal sequence, e.g., from Saccharomyces cerevisiae); which system may be used, for example, in the identification of antibodies. Thus, in an embodiment of the invention, the host cell in the system expresses one or more immunoglobulin chains (e.g., light and heavy chains, e.g., wherein one or more of the chains are from a library source) of an antibody and/or of an Fc/antigen-binding fragment thereof. In an embodiment of the invention, the immunoglobulin chains of an antibody and/or of an Fc/antigen-binding fragment thereof comprises an identical or different CH2-CH3 polypeptide from that of the bait. In particular embodiments, the host cell is constructed by yeast mating.

An Fc/antigen-binding fragment of an antibody (1) complexes with the Fc moiety of the bait and (2) binds to an antigen when complexed with the bait on the surface of the host cell. An example of an Fc/antigen-binding fragment is a monovalent fragment of a full antibody (i.e., a monovalent antibody fragment).

In an embodiment of the invention, the bait comprises a CH2-CH3 polypeptide or functional fragment thereof that differs at one or more residues from the CH2-CH3 of the Fc/antigen-binding fragment of an antibody. In such an embodiment of the invention, when the bait and the Fc/antigen-binding fragment of an antibody bind, a heterodimeric Fc domain is formed.

The “bait” comprises an Fc domain (e.g., human, rat, rabbit, goat or mouse Fc, e.g., any part of the heavy chain (e.g., human, rat, rabbit, goat or mouse) such as, for example, a CH2-CH3 polypeptide optionally further comprising a hinge region) fused, e.g., at the amino-terminus or carboxy-terminus, to a surface anchor comprising more than 320 amino acids, which bait possesses functional properties described herein (e.g., as set forth below) that enable the bait to function in the antibody display system of the present invention. The Fc domain can, in an embodiment of the invention, be mutated so as to improve its ability to function in the antibody display system of the present invention, for example, cysteines or other residues may be added or moved to allow for more extensive disulfide bridges to form when complexed with a human IgG Fc or Fc/antigen-binding fragment. An Fc suitable for use in the bait comprises an Fc or functional fragment thereof (e.g., from an IgG1, IgG2, IgG3 or IgG4 or a mutant thereof) that is capable of dimerizing, when fused to a surface anchor protein, with, for example, a human IgG Fc or with the Fc/antigen-binding fragment on the surface of a eukaryotic host cell. In general, in the absence of the Fc/antigen-binding fragment, the bait homodimerizes, thus, comprising two surface anchors and two Fc domains.

In an embodiment of the invention, a full antibody that is co-expressed with the bait comprises light and heavy chains capable of dimerizing with each other to form a monovalent antibody fragment, which monovalent antibody fragment dimerizes with the Fc of the bait.

In an embodiment of the invention, the surface anchor is any glycosylphosphatidylinositol-anchored (GPI) protein. A functional fragment of a surface anchor comprises a fragment of a full surface anchor poly peptide that is capable of forming a functional bait when fused to an Fc or functional fragment thereof; e.g., wherein the fragment, when expressed in a eukaryotic host cell as a Fc fusion, is located on the cell surface wherein the Fc is capable of forming a complex with an Fc/antigen-binding fragment (e.g., a monovalent antibody fragment).

The scope of the present invention encompasses an isolated Saccharomyces cerevisiae host cell comprising a bait (i.e., comprising the human Fc domain or functional fragment thereof fused, e.g., at the amino-terminus or carboxy-terminus, to the surface anchor or functional fragment thereof of at least 320 amino acids) on the cell surface wherein the bait is dimerized with an Fc/antigen-binding fragment, e.g., by binding between the bait Fc and the heavy chain of a monovalent antibody fragment (e.g., between the CH2-CH3 polypeptides in the bait and the Fc/antigen-binding fragment).

The present invention also includes a composition comprising a Saccharomyces cerevisiae host cell comprising a bait and secreted antibody or antigen-binding fragment thereof and/or Fc/antigen-binding fragment thereof, e.g., in a liquid culture medium.

The present invention provides, for example, a method for identifying (i) an antibody or Fc/antigen-binding fragment thereof that binds specifically to an antigen of interest and/or (ii) a polynucleotide encoding an immunoglobulin heavy chain of said antibody or fragment and/or a polynucleotide encoding an immunoglobulin light chain of said antibody or fragment. The method comprises, in an embodiment of the invention: (a) co-expressing a bait (e.g., comprising a polypeptide comprising a CH3 or CH2-CH3 polypeptide or CH2-CH3 further comprising a hinge region that is linked to a cell surface anchor protein of more than 320 amino acids) and one or more heavy and light immunoglobulin chains (e.g., wherein one or more of such chains are encoded by a polynucleotide from a library source) in an isolated Saccharomyces cerevisiae host cell (such that a complex between the Fc moiety of the bait (e.g., comprising a CH3 or CH2-CH3 polypeptide or CH2-CH3 further comprising a hinge region) and an Fc/antigen-binding fragment (e.g., a monovalent antibody fragment) comprising the immunoglobulin chains forms, and is located at the cell surface; for example, wherein the host cell is transformed with one or more polynucleotides encoding the bait and the immunoglobulin chains: (b) identifying a host cell expressing the bait, dimerized with the Fc/antigen-binding fragment of the antibody (e.g., a monovalent antibody fragment), which has detectable affinity (e.g., acceptable affinity) for the antigen (e.g., which detectably binds to the antigen); for example, wherein the bait, and light and heavy chain immunoglobulins are encoded by the polynucleotides in the eukaryotic host cell;

In an embodiment of the invention, non-tethered, secreted full antibodies comprising light and heavy chain immunoglobulin variable domains identical to those complexed with the bait (e.g., immunoglobulins that are expressed from the host cell) are analyzed to determine if they possess detectable affinity.

In an embodiment of the invention, the full antibodies are secreted from the host cell into the medium. In an embodiment of the invention, the full antibodies are isolated from the host cell.

In an embodiment of the invention, after step (b), expression of the bait in the host cell is inhibited, but expression of the full antibodies is not inhibited. In this embodiment of the invention, the host cell expresses only the full antibody but does not express the bait at any significant quantity. Once expression of the bait is inhibited, in an embodiment of the invention, the full antibody produced from the host cell is analyzed to determine if it possesses detectable affinity (e.g., acceptable affinity); and, (c) identifying said antibodies or antigen-binding fragments or polynucleotides if detectable binding of the Fc/antigen-binding fragment is observed, e.g., wherein one or more of the polynucleotides encoding the light and/or heavy chain immunoglobulin are optionally isolated from the host cell. In an embodiment of the invention, the nucleotide sequence of the polynucleotide is determined.

In an embodiment of the invention, a population of Saccharomyces cerevisiae host cells express a common bait and a common immunoglobulin heavy chain as well a variety of different light chain immunoglobulins, e.g., from a library source, wherein individual light chain immunoglobulins that form Fc/antigen-binding fragments and full antibodies that are tethered to the bait and which exhibit antigen binding can be identified. Similarly, in an embodiment of the invention, a population of said host cells express a common bait and a common immunoglobulin light chain as well a variety of different heavy chain immunoglobulins, e.g., from a library source, wherein individual heavy chain immunoglobulins that form Fc/antigen-binding fragments and full antibodies that are tethered to the bait and which exhibit antigen binding can be identified.

In an embodiment of the invention, the Saccharomyces cerevisiae host cell possessing polynucleotides encoding the heavy and light chain immunoglobulins can be further used to express the secreted non-tethered antibody (e.g., full antibody) or an antigen-binding fragment thereof in culture. For example, in this embodiment of the invention, expression of the bait is optionally inhibited so that bait expression at significant quantities does not occur. The host cell is then cultured in a culture medium under conditions whereby secreted, non-tethered antibody (e.g., full antibody) or antigen-binding fragment thereof is expressed and secreted from the host cell. The non-tethered antibody or antigen-binding fragment thereof can optionally be isolated from the host cell and culture medium. In an embodiment of the invention, the immunoglobulin chains are transferred to a separate host cell (e.g., lacking the antibody display system components) for recombinant expression.

The present invention provides, for example, a method for identifying (i) an antibody or Fc/antigen-binding fragment thereof that binds specifically to an antigen of interest which comprises a second CH2-CH3 that differs from a first CH2-CH3 of a bait at one or more residues or (ii) a polynucleotide encoding an immunoglobulin heavy chain of said antibody or fragment and/or a polynucleotide encoding an immunoglobulin light chain of said antibody or fragment.

The method comprises, in an embodiment of the invention: (a) co-expressing a bait comprising a first CH2-CH3 polypeptide; along with a heavy immunoglobulin chain comprising said second CH2-CH3 polypeptide (e.g., wherein said heavy immunoglobulin chain is from a library source) and a light immunoglobulin chain (e.g., VL-CL), in an isolated Saccharomyces cerevisiae host cell (e.g., Pichia pastoris) such that a complex between the first CH2-CH3 polypeptide of the bait and the second CH2-CH3 polypeptide of a Fc/antigen-binding fragment binds and is located at the cell surface, for example, wherein the host cell is transformed with one or more polynucleotides encoding the bait and the immunoglobulin chains; (b) identifying a host cell expressing the bait, dimerized with the Fc/antigen-binding fragment which has detectable affinity (e.g., acceptable affinity) for the antigen; for example, wherein the bait, and light and heavy chain immunoglobulins are encoded by the polynucleotides in the eukaryotic host cell: and, optionally. (c) identifying said antibodies or antigen-binding fragments or polynucleotides if detectable binding of the Fc/antigen-binding fragment is observed, e.g., wherein one or more of the polynucleotides encoding the light and/or heavy chain immunoglobulin are optionally isolated from the host cell. In an embodiment of the invention, the nucleotide sequence of the polynucleotide is determined.

Bait expression can be inhibited by any of several acceptable means. For example, the polynucleotides encoding the bait (e.g., the surface anchor and/or Fc) can be expressed by a regulatable promoter whose expression can be inhibited in the host cell. In an embodiment of the invention, bait expression is inhibited by RNA interference, anti-sense RNA, mutation or removal of the polynucleotide encoding the bait (e.g., surface anchor and/or Fc) from the host cell or genetic mutation of the polynucleotide so that the host cell does not express a functional bait.

In an embodiment of the present invention, polynucleotides encoding the antibody or Fc/antigen-binding fragment (e.g., monovalent antibody fragment) heavy and light chain are in one or more libraries of polynucleotides that encode light and/or heavy chain immunoglobulins (e.g., one library encoding light chains and one library encoding heavy chains). The particular immunoglobulin chains of interest are, in this embodiment, distinguished from the other chains in the library when the surface-anchored Fc/antigen-binding fragment on the host cell surface is observed to bind to an antigen of interest.

In an embodiment of the invention, the heavy or light chain immunoglobulin expressed in the antibody display system is from a library source and the other immunoglobulin chain is known (i.e., a single chain from a clonal source). In this embodiment of the invention, the antibody display system can be used, as discussed herein, to identify a new library chain that forms desirable antibodies or antigen-binding fragments thereof when coupled with the known chain. Alternatively, the antibody display system can be used to analyze expression and binding characteristics of an antibody or antigen-binding fragment thereof comprising two known immunoglobulin chains.

In an embodiment of the invention, cells expressing Fc/antigen-binding fragments tethered to the cell by an anchor such as SED1 that bind to an antigen can be detected by incubating the cells with fluorescently labeled antigen (e.g., biotin label) and sorting/selecting cells that specifically bind the antigen by fluorescence-activated cell sorting (FACS).

In an embodiment of the invention, the Saccharomyces cerevisiae host cells expressing the bait dimerized with the Fc/antigen-binding fragment are identified and sorted using fluorescence-activated cell sorting (FACS). For example, in an embodiment of the invention, cells expressing the bait dimerized with the Fc/antigen-binding fragment on the cell surface are labeled with a fluorescent antigen or fluorescent secondary antibody that also binds to the antigen. The fluorescent label is detected during the FACS sorting and used as the signal for sorting. Labeled cells indicate the presence of a cell surface expressed bait/Fc/antigen-binding fragment/antigen complex and are collected in one vessel whereas cells not expressing signal are collected in a separate vessel. The present invention, accordingly, includes a method comprising the following steps for determining if an antibody or antigen-binding fragment thereof from a library specifically binds to an antigen:
(1) Transform
   (i) one or more immunoglobulin libraries, containing polynucleotides encoding light and heavy chain immunoglobulins;
   (ii) one or more immunoglobulin libraries, containing polynucleotides encoding light chain immunoglobulins and a single clonal heavy chain immunoglobulin: or one or more immunoglobulin libraries, containing polynucleotides encoding heavy chain immunoglobulins and a single clonal light chain immunoglobulin;
      wherein, said chains are capable of forming an antibody or antigen-binding fragment thereof, into a Saccharomyces cerevisiae host cell comprising polynucleotides encoding the bait;
Grow transformed cells in a liquid culture medium;
(2) Allow expression of the bait on the surface of the cells;
(3) Label the cells with fluorescently labeled antigen or antigen bound to a fluorescently labeled secondary antibody;
(4) Sort and isolate fluorescently labeled cells using FACS for one round;
(5) Regrow the labeled, sorted cells;
(6) Allow expression of the bait in the cells;
(7) Label the cells with fluorescently labeled antigen or antigen bound to a fluorescently labeled secondary antibody;
(8) Sort and isolate fluorescently labeled cells using FACS for a second round;
(9) Regrow the labeled, sorted cells on solid culture medium so that individual cellular clones grow into discrete cellular colonies;
(10) Identify colonies with affinity for the antigen;
(11) Grow cells from identified colonies in a liquid culture medium and isolate supernatant containing full, non-tethered antibody or antigen-binding fragment thereof comprising the immunoglobulin light and heavy chains; wherein, expression of the bait is optionally inhibited;
(12) Determine affinity of non-tethered antibodies or antigen-binding fragments thereof, from the supernatant, for the antigen and identify clones with acceptable affinity (e.g., by SPR or kinetic exclusion assay analysis): and
(13) Determine the nucleotide sequence of polynucleotides in the identified clones encoding the heavy and light chain immunoglobulins.

In an embodiment of the invention, the human Fc immunoglobulin domain for use in the bait comprises an IgG1 Fc domain. In further embodiments, the IgG1 Fc domain lacks an N-glycosylation site, which in particular embodiments may comprise an N297 substitution (position number in accordance with EU numbering), which abolishes the N-glycosylation site beginning at amino acid position 297. For example, an IgG1 Fc comprising an N297A substitution as set forth for the IgG1 Fc amino acid sequence set forth in SEQ ID NO: 22. In an embodiment of the invention, the surface anchor polypeptide comprises between 400 to 700 amino acids.

In particular embodiments, the surface anchor polypeptide comprises a Saccharomyces cerevisiae SED1 protein that has an amino acid sequence greater than 320 amino acids. Exemplary SED1 proteins include naturally occurring Saccharomyces cerevisiae SED1 proteins comprising about 401, 430, or 481 amino acids as disclosed herein and which may have the amino acid sequence set forth in SEQ ID NO: 18, SEQ ID NO: 19, or SEQ ID NO: 20.

In other embodiments, the cell surface anchor polypeptide is a chimeric surface anchor polypeptide comprising a Saccharomyces cerevisiae SED1 protein and a heterologous protein. The chimeric surface anchor polypeptide may comprise a heterologous protein amino acid sequence linked at its N-terminus to the N-terminal portion of a Saccharomyces cerevisiae SED1 protein and at its C-terminus to the C-terminal portion of a Saccharomyces cerevisiae SED1 protein. The heterologous protein amino acid sequence may be a minisatellite-like repeat sequence from a yeast cell wall protein, for example a yeast cell wall protein selected from FLO1, FLO2, and FLO11.

### Regulatable Promoters for Controlling Expression of the Bait and the Antibody Heavy and Light Chains

The present invention uses regulatable promoters for controlling expression of the bait and the antibody heavy and light chains in the host cell. Expression of the bait is under the control of a first regulatable promoter and expression of the antibody heavy and light chains is under control of a second regulatable promoter. The first regulatable promoter enables expression of the bait in the presence of an inducer for the first regulatable promoter while there is trace or no expression of the antibody heavy and light chains under control of the second regulatable promoter. The second regulatable promoter enables expression of the antibody heavy and light chains in the presence of an inducer for the second regulatable promoter while there is trace or no expression of the bait under control of the first regulatable promoter. In particular embodiments, the first and/or second regulatable promoter comprises an inducer/repressor control system in which an activator is bound to positive regulatory elements and an inhibitor is bound to repressor elements in the promoter thereby inhibiting transcription and thus expression from the promoter. For high levels of expression from the promoter, an inducer and a de-repressor are introduced into the host cell: the de-repressor removes the inhibitor and the inducer activates transcription and thus expression. In particular embodiments, the first regulatable promoter is under the control of an inducer that promotes transcription from the promoter without the need for a de-repressor and the second regulatable promoter is under the control of an inducer/repressor system.

Examples of regulatable promoters include promoters from numerous species, including but not limited to alcohol-regulated promoter, tetracycline-regulated promoters, steroid-regulated promoters (e.g., glucocorticoid, estrogen, ecdysone, retinoid, thyroid), metal-regulated promoters, pathogen-regulated promoters, temperature-regulated promoters, and light-regulated promoters. Specific examples of regulatable promoter systems well known in the art include but are not limited to metal-inducible promoter systems (e.g., the yeast copper-metallothionein promoter), plant herbicide safner-activated promoter systems, plant heat-inducible promoter systems, plant and mammalian steroid-inducible promoter systems, Cym repressor-promoter system (Krackeler Scientific, Inc. Albany, NY), RheoSwitch System (New England Biolabs, Beverly MA), benzoate-inducible promoter systems (See WO2004/043885), and retroviral-inducible promoter systems. Other specific regulatable promoter systems well-known in the art include the tetracycline-regulatable systems (See for example, Berens & Hillen, Eur J Biochem 270: 3109-3121 (2003)), RU 486-inducible systems, ecdysone-inducible systems, and kanamycin-regulatable system. Lower eukaryote-specific promoters include but are not limited to the Saccharomyces cerevisae TEF-1 promoter, Pichia pastoris GAPDH promoter. Pichia pastoris GUT1 promoter, PMA-1 promoter, Pichia pastoris PCK-1 promoter, and Pichia pastoris AOX-1 and AOX-2 promoters. For temporal expression of the GPI-IgG capture moiety and the immunoglobulins, the Pichia pastoris GUT7 promoter operably linked to the nucleic acid molecule encoding the GPI-IgG capture moiety and the Pichia pastoris GAPDH promoter operably linked to the nucleic acid molecule encoding the immunoglobulin are shown in the examples herein to be useful. Examples of transcription terminator sequences include transcription terminators from numerous species and proteins, including but not limited to the Saccharomyces cerevisiae cytochrome C terminator; and Pichia pastoris ALG3 and PMA1 terminators.

In particular embodiments, the first regulatable promoter is a TetO7 promoter, which is activatable in the presence of doxycycline, and the second regulatable promoter is a GAL1 promoter. The GAL1 promoter is an inducible/repressor promoter. The promoter contains both negative and positive regulatory sites encoded within its DNA sequence. In the presence of glucose, repressor proteins bind to the negative regulatory sites and repress transcription. The Gal4p transcriptional activator binds to positive regulatory sites. Gal4p is a transcription factor that binds to DNA as a dimer. In the presence of glucose, Gal4p is inactive, because it is bound to the repressor protein, Gal80p. Glucose repression can be relieved by growing cells in a poor carbon source, such as raffinose. Raffinose is a trisaccharide composed of galactose, fructose and glucose. Raffinose is not able to induce high levels of GAL1 expression, which requires galactose. In the presence of galactose, expression of the GAL1 gene increases about 1000-fold above the level observed in the presence of glucose. This stimulation is primarily due to the activity of Gal4p, which is no longer bound to the inhibitory Gal80p protein. In particular embodiments, the first regulatable promoter is a GAL1 promoter and the second regulatable promoter is a TetO7 promoter, which is activatable in the presence of doxycycline.

### Controlling O-Glycosylation

In particular embodiments, the yeast host cells are genetically modified to control O-glycosylation of the glycoprotein by deleting or disrupting one or more of the protein O-mannosyltransferase (Dol-P-Man:Protein (Ser/Thr) Mannosyl Transferase genes) (PMTs) (See for example U.S. Pat. No. 5,714,377) or grown in the presence of one or more Pmtp inhibitors as disclosed in U.S. Pat. Nos. 8,206,949, 7,105,554, or 8,309,325. In particular embodiments, the host cell is genetically modified to lack or have reduced expression of one or more PMTs and grown in the presence of one or more Pmtp inhibitors. In a further embodiment, the host cell may further include a nucleic acid molecule encoding an α-mannosidase. Disruption includes disrupting the open reading frame encoding the Pmtp or disrupting expression of the open reading frame or abrogating translation of RNAs encoding one or more of the Pmtps using interfering RNA, antisense RNA, or the like. The host cells can further include any one of the aforementioned host cells modified to produce particular N-glycan structures.

Pmtp inhibitors include but are not limited to a benzylidene thiazolidinediones. Examples of benzylidene thiazolidinediones that can be used are 5-[[3,4-bis(phenylmethoxy) phenyl]methylene]-4-oxo-2-thioxo-3-thiazolidineacetic Acid; 5-[[3-(1-Phenylethoxy)-4-(2-phenylethoxy)]phenyl]methylene]-4-oxo-2-thioxo-3-thiazolidineacetic Acid; and 5-[[3-(1-Phenyl-2-hydroxy)ethoxy)-4-(2-phenylethoxy)]phenyl]methylene]-4-oxo-2-thioxo-3-thiazolidineacetic acid.

In particular embodiments, the function or expression of at least one endogenous PMT gene is reduced, disrupted, or deleted. For example, in particular embodiments the function or expression of at least one endogenous PMT gene selected from the group consisting of the PMT1, PMT2, PMT3, and PMT4 genes is reduced, disrupted, or deleted: or the host cells are cultivated in the presence of one or more PMT inhibitors. In further embodiments, the host cells include one or more PMT gene deletions or disruptions and the host cells are cultivated in the presence of one or more Pmtp inhibitors. In particular aspects of these embodiments, the host cells also express a secreted α-1,2-mannosidase.

PMT deletions or disruptions and/or Pmtp inhibitors control O-glycosylation by reducing O-glycosylation occupancy, that is, by reducing the total number of O-glycosylation sites on the glycoprotein that are glycosylated, the further addition of an α-1,2-mannosidase that is secreted by the cell controls O-glycosylation by reducing the mannose chain length of the O-glycans that are on the glycoprotein. Thus, combining PMT deletions or disruptions and/or Pmtp inhibitors with expression of a secreted α-1,2-mannosidase controls O-glycosylation by reducing occupancy and chain length. In particular circumstances, the particular combination of PMT deletions or disruptions, Pmtp inhibitors, and α-1,2-mannosidase is determined empirically as particular heterologous glycoproteins (Fabs and antibodies, for example) may be expressed and transported through the Golgi apparatus with different degrees of efficiency and thus may require a particular combination of PMT deletions or disruptions, Pmtp inhibitors, and α-1,2-mannosidase. In another aspect, genes encoding one or more endogenous mannosyltransferase enzymes are deleted. This deletion(s) can be in combination with providing the secreted α-1,2-mannosidase and/or PMT inhibitors or can be in lieu of providing the secreted α-1,2-mannosidase and/or PMT inhibitors.

Thus, the control of O-glycosylation can be useful for producing particular glycoproteins in the host cells disclosed herein in better total yield or in yield of properly assembled glycoprotein. The reduction or elimination of O-glycosylation appears to have a beneficial effect on the assembly and transport of whole antibodies and Fab fragments as they traverse the secretory pathway and are transported to the cell surface. Thus, in cells in which O-glycosylation is controlled, the yield of properly assembled antibodies or Fab fragments is increased over the yield obtained in host cells in which O-glycosylation is not controlled.

The following examples provide particular embodiments of the present invention and to promote a further understanding of the present invention.

### GENERAL METHODS

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, New York (herein “Sambrook, et al., 1989”): DNA Cloning: A Practical Approach, Volumes I and II (D. N. Glover ed. 1985); Oligonucleotide Synthesis (M. J. Gait ed. 1984); Nucleic Acid Hybridization (B. D. Hanes & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel, et al. (eds.), Current Protocols in Molecular Biology, John Wiley & Sons, Inc. (1994).

Methods for protein purification including immunoprecipitation, chromatography, electrophoresis, centrifugation, and crystallization are described (Coligan, et al (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, MO; pp. 45-89: Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, NY: Harlow and Lane, supra). Standard techniques for characterizing ligand/receptor interactions are available (see, e.g., Coligan, et al. (2001) Current Protocols in Immunology, Vol. 4, John Wiley, Inc., New York).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, NJ; Givan (2001) Flow Cytometry, 2nd ed.; Wiley-Liss, Hoboken, NJ; Shapiro (2003) Practical Flow Cytometry, John Wiley and Sons, Hoboken, NJ). Fluorescent reagents suitable for modifying nucleic acids, including nucleic acid primers and probes, polypeptides, and antibodies, for use, e.g., as diagnostic reagents, are available (Molecular Probes (2003) Catalogue, Molecular Probes, Inc., Eugene, OR; Sigma-Aldrich (2003) Catalogue, St. Louis, MO).

Standard methods of histology of the immune system are described (see, e.g., Muller-Harmelink (ed.) (1986) Human Thymus: Histopathology and Pathology, Springer Verlag, New York, NY; Hiatt, et al. (2000) Color Atlas of Histology, Lippincott, Williams, and Wilkins, Phila, PA: Louis, et al. (2002) Basic Histology: Text and Atlas, McGraw-Hill, New York, NY).

Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, MD). GCG Wisconsin Package (Accelrys. Inc., San Diego, CA); DeCypher® (TimeLogic Corp., Crystal Bay, Nevada); Menne, et al. (2000) Bioinformatics 16: 741-742; Menne, et al. (2000) Bioinformatics Applications Note 16:741-742: Wren, et al. (2002) Comput. Methods Programs Biomed. 68:177-181; von Heijne (1983) Eur. J. Biochem. 133:17-21: von Heijne (1986) Nucleic Acids Res. 14:46834690).

### Example 1

Construction and testing SED1.320 mediated Fc display in S. cerevisiae host cells: The antibody display system comprises (i) a nucleic acid molecule encoding an antibody heavy chain variable domain fused to a heavy chain constant domain (HC), (ii) a nucleic acid molecule encoding an antibody light chain variable domain fused to a light chain constant domain (LC), and (iii) a nucleic acid molecule encoding a surface display bait comprising an Fc molecule fused at the C-terminus to a cell surface glycophosphatidylinositol (GPI) anchor protein that enables efficient display of the Fc molecule tethered by the GPI anchor protein to the outer surface of the S. cerevisiae host cell. S. cerevisiae host cells comprising nucleic molecules encoding the HC and LC are induced to express the encoded HC and LC polypeptides, which are then assembled into HC/LC heterodimer pairs (monovalent antibody fragment (H+L)) and antibody tetramers (bivalent antibody (2H+2L)), both of which are secreted from the host cell into the cell culture medium. Outside the host cell, secreted HC/LC heterodimer pairs may also be assembled into antibody tetramers (bivalent antibody (2H+2L)). Concurrently with expression and secretion of the monovalent antibody fragment (H+L), the host cells are also induced to express the cell surface-anchored bait in which the Fc portion thereof can associate with the Fc of a secreted monovalent antibody fragment (H+L) to form a heterotrimer (Bait Fc:HC:LC) in which the monovalent antibody fragment (H+L) thereof is displayed on the cell surface thereof and is capable of binding a protein of interest. Host cells that express antibodies that bind a protein of interest can be identified by labeling the protein of interest with a detectable moiety and then selecting those host cells that display a monovalent antibody fragment (H+L) on the cell surface that binds the labeled protein of interest from those host cells that do not bind the labeled protein of interest using cell sorting methods.

The surface display bait expression cassette was constructed as follows. A nucleic acid molecule encoding a cell surface glycophosphatidylinositol (GPI) anchor protein was linked to a nucleic acid molecule that encodes the human IgG1 Fc domain. For the anchor protein, we used the S. cerevisiae Sed1.320 protein, which is 320 amino acids long and which was successfully used as the anchor to display antibodies in Pichia pastoris yeast display system such as described in U.S. Pat. No. 9,365,846.

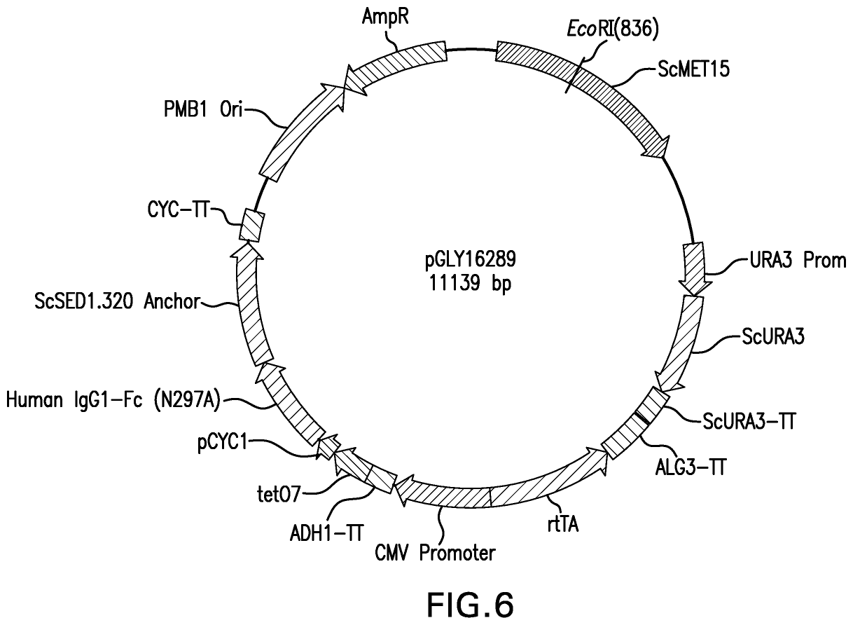

The Fc-SED1.320 bait expression cassette (SEQ ID NO: 1) for expression in S. cerevisiae was constructed as follows. A codon optimized nucleic acid molecule encoding a human IgG1 Fc N297A mutein was synthesized to have at the 5′ end of the nucleic acid molecule an AfeI restriction enzyme site followed by a nucleic acid molecule encoding the S. cerevisiae α-mating factor pre signal sequence (SEQ ID NO: 25) in-frame with the open reading frame (ORF) for the Fc N297A mutein (SEQ ID NO: 22) and at the 3′ end of the nucleic acid molecule encoding the Fc N297A mutein, a nucleic acid molecule encoding in-frame with the ORF encoding the Fc N297A mutein a glycine-rich linker (SEQ ID NO: 23) followed by a nucleic acid molecule encoding in-frame the SED1.320 anchor protein (SEQ ID NO: 17) and ending with an FseI restriction enzyme site on the 3′ end. The Fc-SED1.320 bait cassette was ligated into an in-house created yeast doxycycline-inducible TetO7 promoter plasmid using AfeI and FseI restriction enzyme sites to generate plasmid pGLY16289 in which the Fc-SED1.320 bait expression cassette is operably linked to the TetO7 promoter (SEQ ID NO: 2; See FIG. 6) at the 5′ end and the CYC-TT (CYC transcription termination sequence) at the 3′ end (SEQ ID NO: 3). The TetO7 promoter allows temporal control of Fc-SED1.320 bait expression (see for example Wishart et al., Yeast 23(4): 325-31 (2006)).

As shown in FIG. 6, plasmid pGLY16289 contains the S. cerevisiae MET15 gene (SEQ ID NO: 4), which serves as an integration locus in the genome for the Fc-SED1.320 bait expression cassette, and the URA3 resistance gene (SEQ ID NO: 5), to allow selection of cells expressing the Fc-SED1.320 bait in media without uracil.

To test the capability of the Fc-SED1.320 bait to display human Fc fragment on the yeast cell wall, plasmid pGLY16289 was introduced into the parental yeast strain BJ5465 (Mating Type a ura3-52 trp1 leu2Δ1 his3Δ200 pep4::HiS3 prb1 Δ1.6R can1 GAL, ATCC 208289). Yeast host cells were cultivated in YPD (yeast extract peptone dextrose) media at 30° C. overnight with 1 μg/mL, 3 μg/mL, 5 μg/mL, or 10 μg/mL doxycycline concentration to induce SED1.320 bait expression. Cells were labeled with allophycocyanin (APC)-conjugated goat polyclonal F(ab′)₂ anti-human IgG Fcγ fragment specific detection reagent, which detects human Fc polypeptides, and were processed by flow cytometry for cell sorting. Briefly, each culture, after growing overnight to saturation, two optical density (OD) units of cells, at 600 nm, were pelleted by centrifugation and the cell pellets washed in 1000 μL phosphate buffer saline (PBS)+0.1% bovine serum albumin (BSA). Washed cells were then incubated for 30 minutes at room temperature (RT) in 200 μL PBS-BSA containing APC-conjugated goat polyclonal F(ab′)₂ anti-human IgG Fcγ fragment specific detection reagent and washed twice in 500 μL PBS-BSA. Two hundred microliters of PBS-BSA was used to resuspend pellets before analyzing in a flow cytometer.

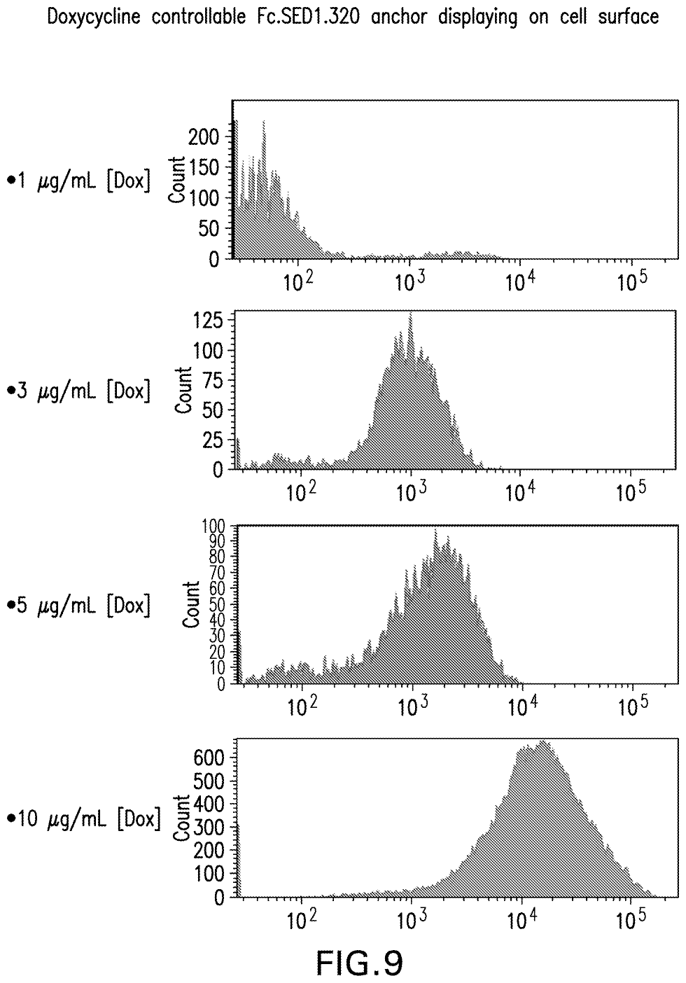

FIG. 9 shows that an increasing level of fluorescence intensity of the Fc detection reagent was observed as the doxycycline concentration was increased, thus indicating an increasing amount of expression of the Fc-SED1.320 bait. The results indicate SED1.320 anchor is able to display the human IgG1 Fc fragment on the cell surface.

### Example 2

Generation of antibody HC and LC diploid yeast by mating: To establish the utility of Fc-SED1.320 bait method for displaying monovalent antibody fragments (H+L) on the yeast cell surface, we tested the Fc-SED1.320 bait method to capture an anti-Her2 monovalent antibody fragment (H+L) comprising the trastuzumab heavy chain variable region (VH) fused to a human IgG1 Fc constant region with the N297A mutation and trastuzumab light chain variable region (VL) fused to the human kappa LC constant region.

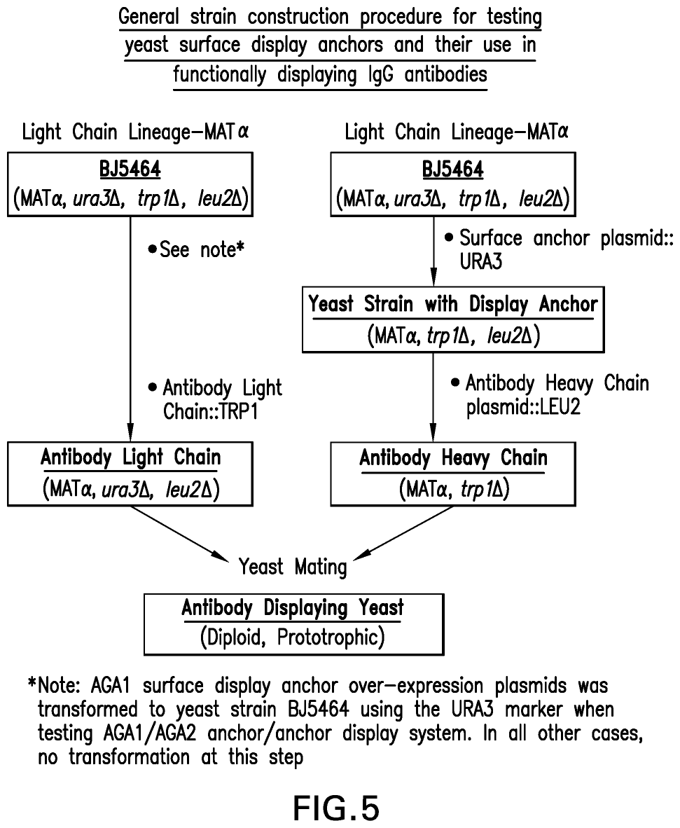

FIG. 5 shows a general strain construction procedure for yeast surface display anchors and their use in functionally displaying IgG antibodies.

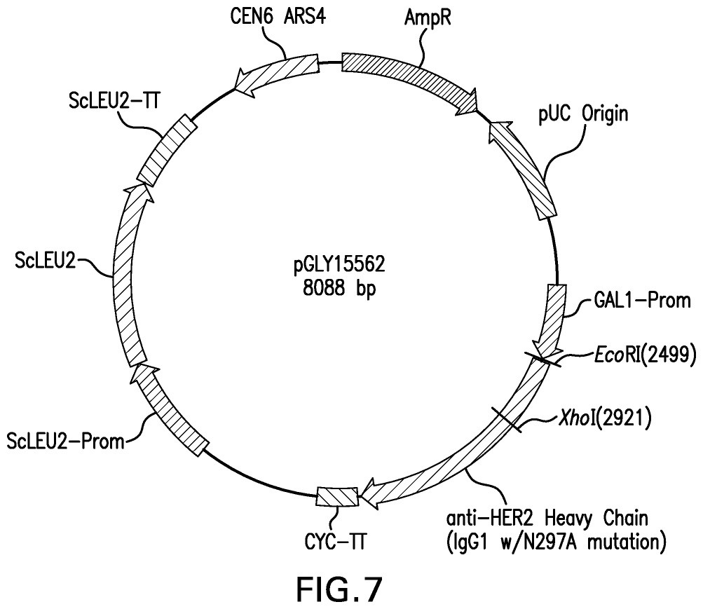

Plasmid pGLY15562 (FIG. 7) comprising a trastuzumab HC expression cassette was constructed and transformed into Fc-SED1.320 expressing BJ5465 yeast cells (mating type a) constructed as described in Example 1 to produce haploid yeast cells that express the trastuzumab HC. The trastuzumab expression cassette comprises a nucleic acid molecule encoding the trastuzumab VH domain with a S. cerevisiae α-mating factor pre signaling sequence fused to its 5′ end and an IgG1 Fc comprising the N297A mutation fused to its 3′ end (SEQ ID NO: 6) operably linked to an S. cerevisiae GAL1 promoter (SEQ ID NO: 7) at the 5′ end and the CYC-TT at the 3 end. Plasmid pGLY15562 also contains the S. cerevisiae LEU2 open reading frame (ORF) set forth in SEQ ID NO: 8 to allow selection in media without leucine. The trastuzumab HC-expressing haploid yeast cells were thus both uracil and leucine prototrophic and tryptophan auxotrophic.

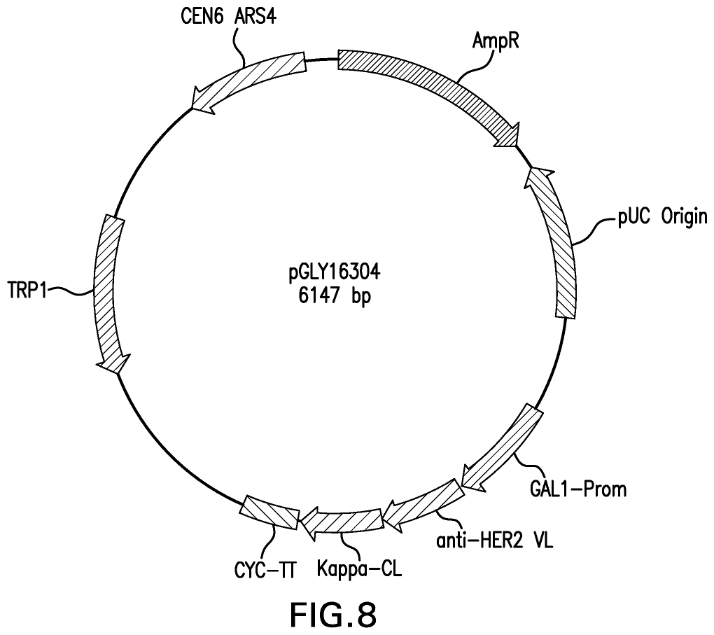

Plasmid pGLY16304 (FIG. 8) comprising a trastuzumab LC expression cassette was constructed and transformed into the isogenic parental yeast strain S. cerevisiae BJ5464 (Mating type α ura3-52 trp1 leu2Δ1 his3Δ200 pep4::HIS3 prb1Δ1.6R can1 GAL, ATCC 208288). The trastuzumab LC expression cassette comprises a nucleic acid molecule encoding the trastuzumab VL domain with a S. cerevisiae α-mating factor pre signaling sequence fused to its 5′ end and the human kappa LC constant domain fused to its 3′ end (SEQ ID NO: 9) operably linked to an S. cerevisiae GAL1 promoter at the 5′ end and the CYC-TT at the 3′ end. Plasmid pGLY16304 also contained the S. cerevisiae TRP1 ORF set forth in SEQ ID NO: 10 to allow selection in media without tryptophan. The trastuzumab LC-expressing haploid yeast cells were therefore tryptophan prototrophic and uracil and leucine auxotrophic.

To create mated diploid cells that express both the trastuzumab HC and the trastuzumab LC, the trastuzumab HC-expressing haploid yeast cells and the trastuzumab LC-expressing haploid yeast cells were mated to form diploid cells (See FIG. 5). Briefly, the yeast haploid cells were inoculated to grow in respective amino acid drop out selective media at 30° C., 220 rpm overnight. The starting OD of the culture was 0.1 at 600 nm. On the second day, equal amounts of trastuzumab-expressing HC and trastuzumab-expressing LC haploid cells were combined, pelleted by centrifugation, and the cell pellet resuspended in a small amount of YPD media, which was then plated on 150 mm YPD agar plates at a density of 3×10⁷ cells/cm². The plates were incubated 30° C. for six hours to allow mating. Afterwards, cells were scraped down gently from plates and the diploid cells were selected by growing cells on media without leucine, tryptophan, and uracil.

### Example 3

Growth and induction of antibody HC and LC expression: To induce antibody expression on the yeast surface, diploid yeast cells were first grown in 4% glucose dropout media lacking leucine, tryptophan, and uracil overnight at 30° C., at a density less than 0.5 OD/mL at 600 nm. Cells were then switched to de-repression/induction medium (dropout media containing 4% raffinose and 10 μg/mL doxycycline) at a starting OD of 0.5 OD/mL at 600 nm and grown overnight at 30° C. to de-repress the GAL1 promoter and induce expression the Fc-SED1.320 bait. The following morning, the cells were centrifuged at 3,600 g for three minutes and the cell pellet was resuspended into induction media (dropout media containing 2% raffinose, 2% galactose, and 10 μg/mL doxycycline) at an OD of 1.0 to induce expression of the trastuzumab HC and trastuzumab LC under control of the GAL1 promoter. Induction media was supplemented with an O-linked glycosylation inhibitor at a final concentration of 1.8 mg/L (see U.S. Pat. No. 8,309,325). The cells were induced at 24° C. for 24 hours with shaking at 220 rpm.

### Example 4

Testing Fc-SED1.320 mediated monovalent antibody fragment (H+L) display in yeast: To determine the efficiency and functionality of surface display of a monovalent antibody fragment (H+L) using the Fc-SED1.320 anchor, cells were labeled with Goat F(ab′)₂ anti-human Kappa-Alexa Fluor 647, which detects the LC kappa constant domain of human antibody molecules, and a biotinylated soluble HER2 antigen.

Briefly, five OD units of diploid cells from Example 3 at 600 nm were centrifuged and the cell pellet was washed 2× with phosphate buffered saline containing 0.1% bovine serum albumin (PBS-BSA). Cells were incubated with 100 nM antigen in a total volume of 200 μL for one hour at 30° C. shaking, then washed again 2× with PBS-BSA. Next, cells were incubated with three secondary detection reagents: Goat F(ab′)₂ anti-human Kappa-Alexa Fluor 647 (Southern Biotech) to detect trastuzumab LC expression, NeutrAvidin conjugated to R-phycoerythrin (PE) (ThermoFisher) to detect binding of the biotinylated Her2 antigen, and YOYO1 nuclear dye (ThermoFisher) to measure cell viability. The three secondary detection reagents were added at a dilution of 1:100, 1:200, and 1:2000, respectively, in a total volume of two mL, and incubated for 30 minutes on ice. After secondary incubation, cells were washed 2× with PBS-BSA and resuspended in PBS-BSA for FACS analysis.

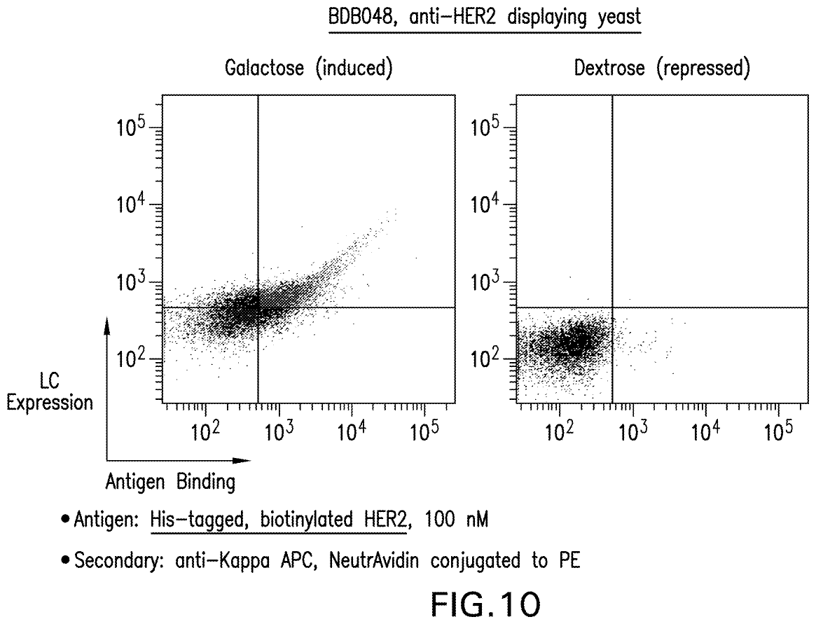

FIG. 10 shows flow cytometry analysis of Fc-SED1.320 bait mediated anti-Her2 display on the cell surface. Controls were prepared by growing cells in dextrose media in which trastuzumab HC and LC expression was repressed but Fc-SED1.320 protein expression was induced. In FIG. 10, the fluorescent intensities suggested the trastuzumab LC expression was very low (Y-axis). Interestingly, the high trastuzumab LC signal cell population indicated that HER2 antigen was being bound. While the data suggested that the displayed anti-trastuzumab monovalent antibody fragment (H+L) was functional since it bound HER2 antigen, the very low trastuzumab LC expression and population indicated that antibody display based on Fc.SED1-320 would not be suitable for large-scale antibody library sorting applications.

### Example 5

Testing Fc-AGA2/AGA1 mediated monovalent antibody fragment (H+L) display in yeast: Since the performance of the Fc-SED1.320 mediated antibody display was unsatisfactory for large-scale antibody library sorting applications, efforts were initiated to identify alternative surface protein anchors that would enable efficient monovalent antibody fragment (H+L) display in S. cerevisiae. We tested the yeast AGA1/AGA2 cell surface display system, a commonly used yeast anchor system for display applications.

Unlike the SED1.320 anchor that only consists of a single SED1 polypeptide, functioning as both anchor and carrier of the Fc, the AGA1 and AGA2 function as surface anchor and heterologous protein carrier, respectively. In the AGA1-AGA2 cell surface display system, the heterologous protein of interest (e.g., Fc bait) is expressed as a fusion protein that is fused to the N-terminus of the AGA2 mating agglutinin protein, which is then covalently linked to the AGA1 on the cell wall by two disulfide bonds.

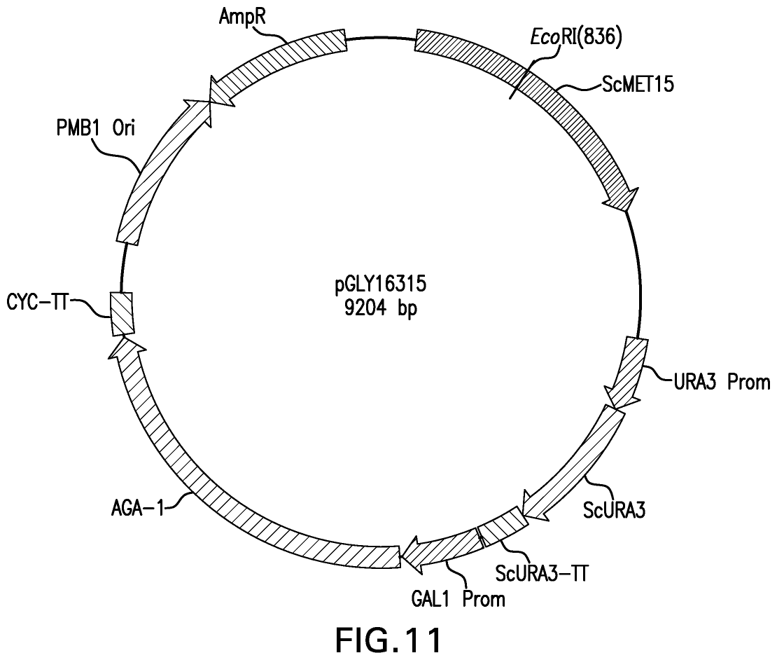

To test the Fc-AGA2 bait mediated monovalent antibody fragment (H+L) display in yeast, both AGA1 anchor protein and Fc-AGA2 bait were overexpressed. Plasmid pGLY16315 (FIG. 11) encodes the yeast AGA1 open reading frame (ORF: (SEQ ID NO: 11) under the control of the galactose-inducible GAL1 promoter at the 5′ end and the CYC-TT at the 3′ end. Plasmid pGLY16315 contains the S. cerevisiae MET15 gene, which served as an integration locus in the genome, and the URA3 resistance gene, to allow selection on media without uracil. Plasmid pGLY16315 was linearized using EcoRI restriction enzyme and transformed into yeast B35464.

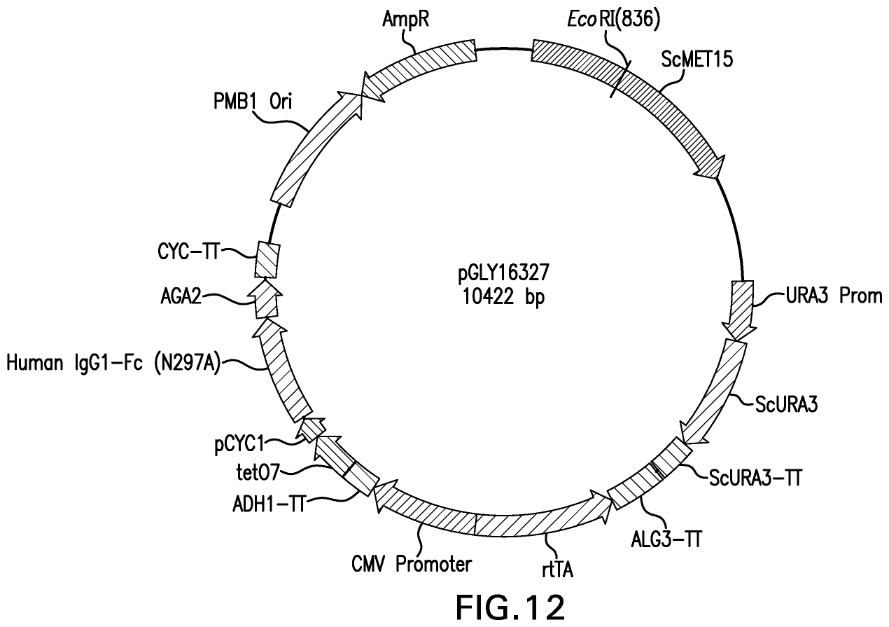

Plasmid pGLY16327 (FIG. 12) contains an Fc-AGA2 bait expression cassette. A codon optimized nucleic acid molecule encoding human IgG1 Fc N297A mutein was synthesized having an AfeI restriction enzyme site at the 5′end followed by the nucleic acid molecule encoding the S. cerevisiae α-mating factor signal sequence with the nucleic acid molecule encoding the human IgG1 Fc followed by a glycine-rich linker, a nucleic acid molecule encoding the AGA2 carrier protein, and FseI restriction enzyme site at the 3 end (SEQ ID NO: 12). The insert was ligated to an in-house created yeast doxycycline inducible TetO7 promoter plasmid using AfeI and FseI restriction enzyme sites to generate plasmid pGLY16327 in which the expression cassette is operably linked to the TetO7 promoter at the 5′ end and the CYC-TT at the 3′ end. Plasmid pGLY16327 contains the S. cerevisiae MET15 gene sequence, which serves as an integration locus in the genome, and the URA3 resistance gene, to allow selection on media without uracil. Plasmid pGLY16327 was linearized using EcoRI enzyme and transformed to yeast BJ5465.

Anti-Her2 HC plasmid pGLY15562 (FIG. 7) was transformed into the Fc-AGA2 over-expressing BJ5465 yeast cells. Anti-Her2 LC plasmid pGLY16304 (FIG. 8) was transformed into the AGA1 over-expressing BJ5464 Strains. The two strains were mated following the procedure described in Example 2 (see also FIG. 5) to form Fc-AGA2/AGA1 mediated monovalent anti-HER2 HC and LC diploid cells.

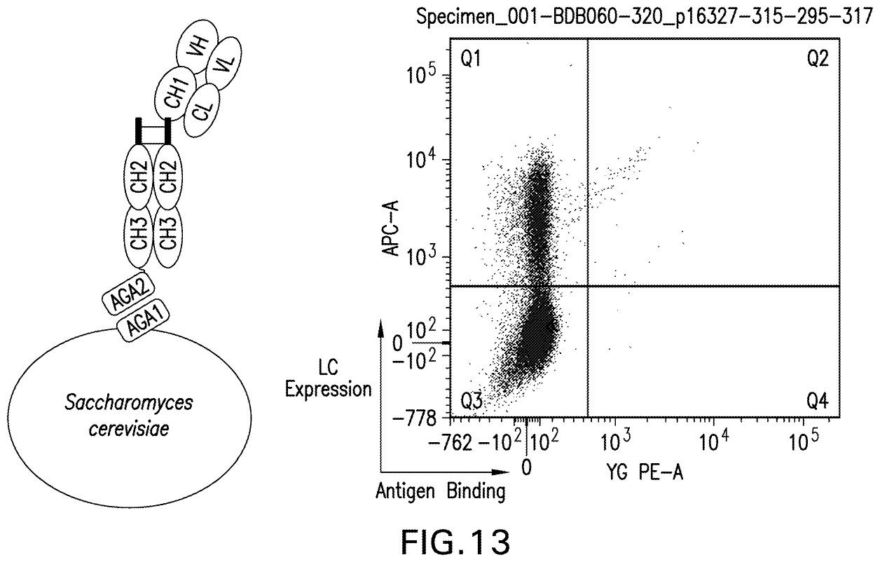

FIG. 13 shows flow cytometry analysis of Fc-AGA2/AGA1 bait mediated anti-Her2 monovalent antibody fragment (H+L) displayed on cell surface. Goat F(ab′)₂ anti-human Kappa-Alexa Fluor 647 was used to detect LC expression (Y-axis) and NeutrAvidin conjugated to PE (ThermoFisher) was used to detect biotinylated HER2 binding (X-axis). The fluorescent intensities of polyclonal Goat F(ab′)₂ anti-human Kappa signal suggested the LC expression level was high but the majority of the monovalent antibody fragment (H+L) was not functional and did not bind HER2 antigen.

### Example 6

Identification of naturally occurring long S. cerevisae SED1 Protein. While the Fc-SED1.320 bait mediated monovalent antibody fragment (H+L) display system works efficiently in methylotrophic yeast Pichia pastoris (See for example U.S. Pat. No. 9,365,846), as shown in Example 4, it functioned poorly in S. cerevisiae with very little LC expression. The results in Example 5 shows the AGA1/AGA2 bait bound high levels of LC on the cell surface indicating high amounts of LC was being expressed but the resulting displayed monovalent antibody fragment (H+L) was non-functional. In S. cerevisiae, the most commonly used AGA1 protein anchor is 675 amino acids in length. We postulated that the SED1.320 protein may not be long enough to functionally display monovalent antibody fragment (H+L) on the surface of S. cerevisiae. We searched genome databases and analyzed SED1 gene lengths and sequence polymorphisms to identify S. cerevisiae SED1 alleles that had a longer amino acid length than SED1.320.

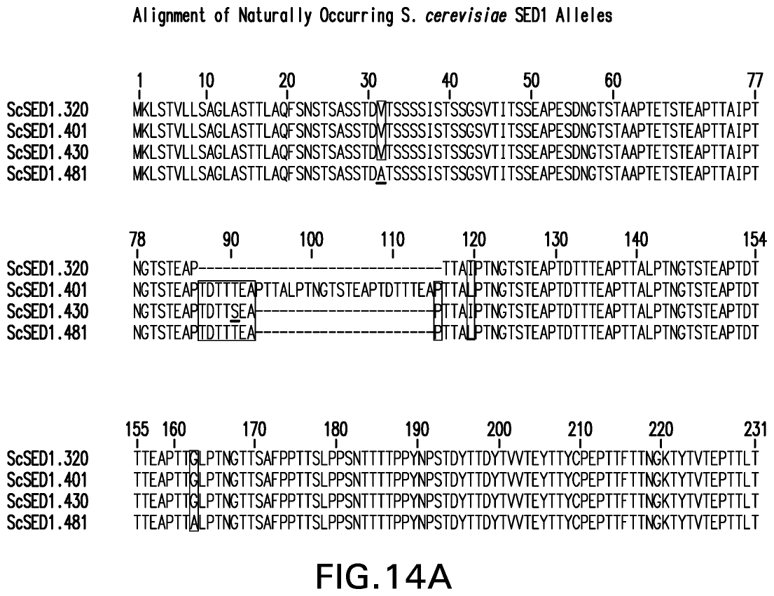

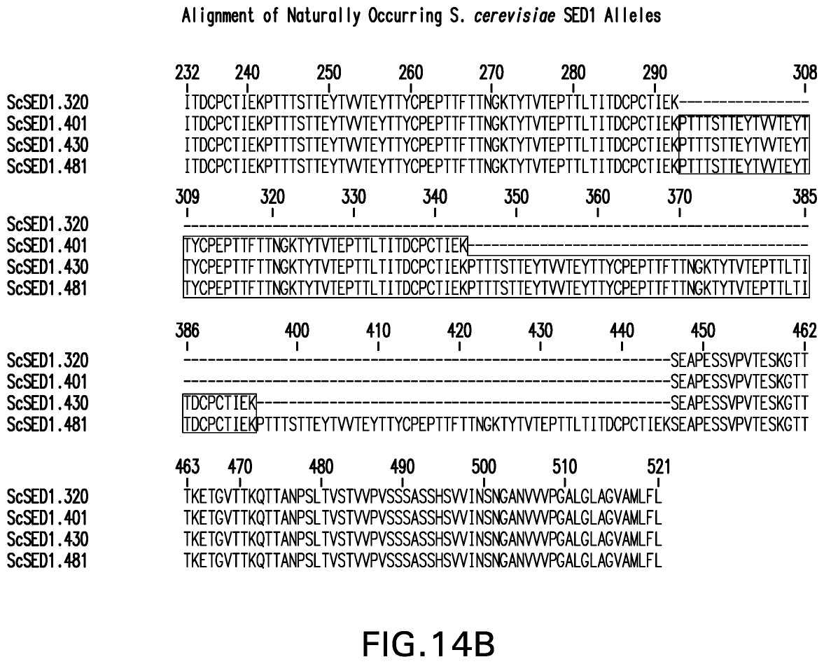

FIG. 14A and FIG. 14B show a sequence alignment of several naturally occurring S. cerevisiae SED1 alleles that are 320 amino acid or longer in length. Sequence analysis suggested presence of polymorphic minisatellite-like tandem repeat sequences, variable in length, within the SED1 open reading frame (ORF). The longest naturally occurring SED1 allele identified is 481 amino acids long excluding signal peptide. This specific SED1 protein is referred to herein as SED1.481 (SEQ ID NO: 20).

### Example 7

Testing Fc-SED1.481 mediated monovalent antibody fragment (H+L) display in yeast. We tested the longest SED1 variant, SED1.481, for its ability to display functional monovalent antibody fragment (H+L) in yeast.

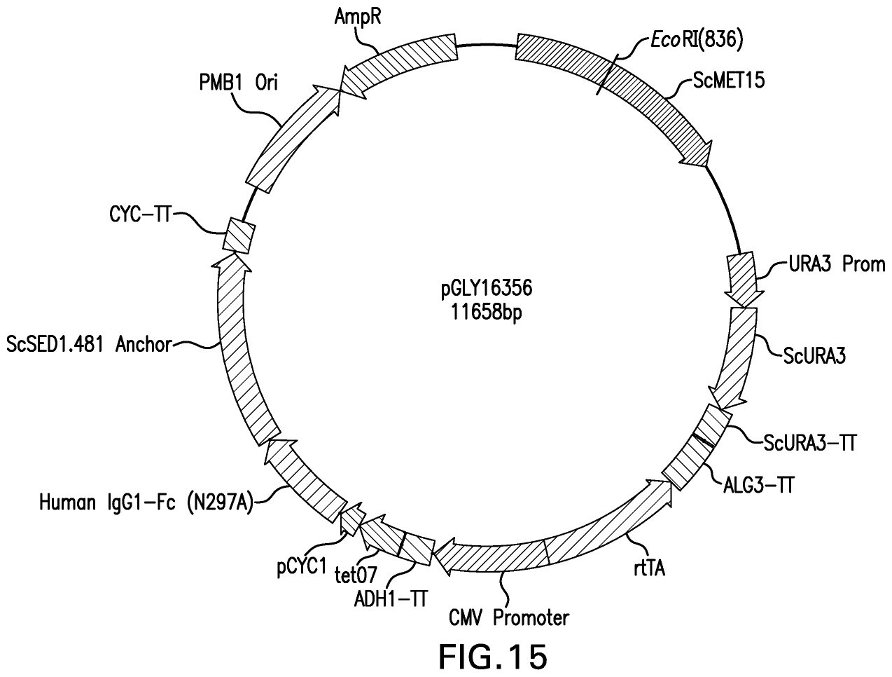

FIG. 15 shows the map of pGLY16356, which contains a doxycycline inducible TetO7 promoter-driven Fc-SED1.481 bait expression cassette (SEQ ID NO:31). Plasmid pGLY16356 was constructed in the same way as previously described or the pGLY16289 vector except the Fc-SED1.320 bait expression cassette in pGLY16289 vector was replaced by the Fc-SED1.481 expression cassette. Plasmid pGLY16356 contains the S. cerevisae MET15 gene sequence, which serves as an integration locus in the genome, and the URA3 resistance gene, to allow selection in media without uracil.

Plasmid pGLY16356 was transformed into BJ5465 to generate an Fc-SED1.481 bait strain. The anti-HER2 HC plasmid pGLY15562 was transformed into Fc-SED1.481 containing BJ5465 yeast cells. Then, the anti-HER2 LC plasmid pGLY16304 was transformed into the isogenic parental yeast strain S. cerevisiae BJ5464. Anti-HER2 displaying diploid yeast was obtained by mating the haploid yeast cells as described in Example 2 and illustrated in FIG. 5. Detection and function of displayed the monovalent antibody fragment (H+L) was determined as described in the previous examples.

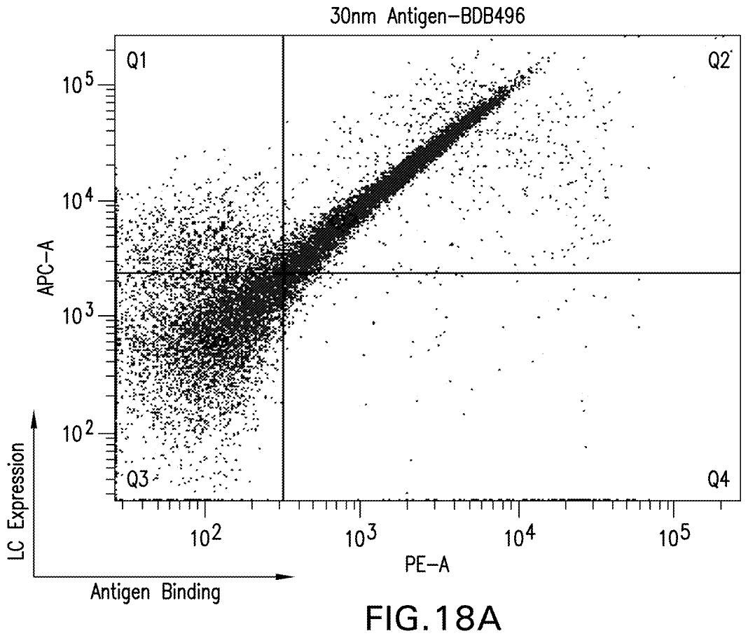

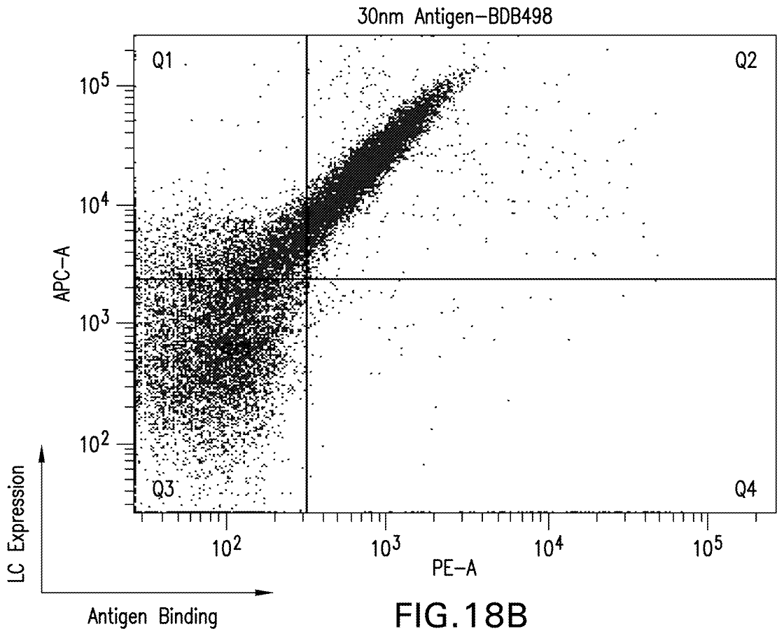

FIG. 18A shows flow cytometry analysis of Fc-SED1.481 bait mediated anti-Her2 monovalent antibody fragment (H+L) displayed on the cell surface. The fluorescent profiles suggested the LC expression level was high (Goat F(ab′)₂ anti-human Kappa-Alexa Fluor 647, Y-axis) and the displayed anti-HER2 monovalent antibody fragments (H+L) were fully functional (X-axis, NeutrAvidin conjugated to PE plus biotinylated human HER2 Ectodomain antigen).

To confirm the utility of Fc-SED1.481 bait to display other antibodies, we selected the anti-TNFα antibody adalimumab as a second test case. The adalimumab VH was fused to the human IgG1 Fc constant domain with an N297A mutation and the adalimumab VL was fused to the human kappa LC constant domain as follows.

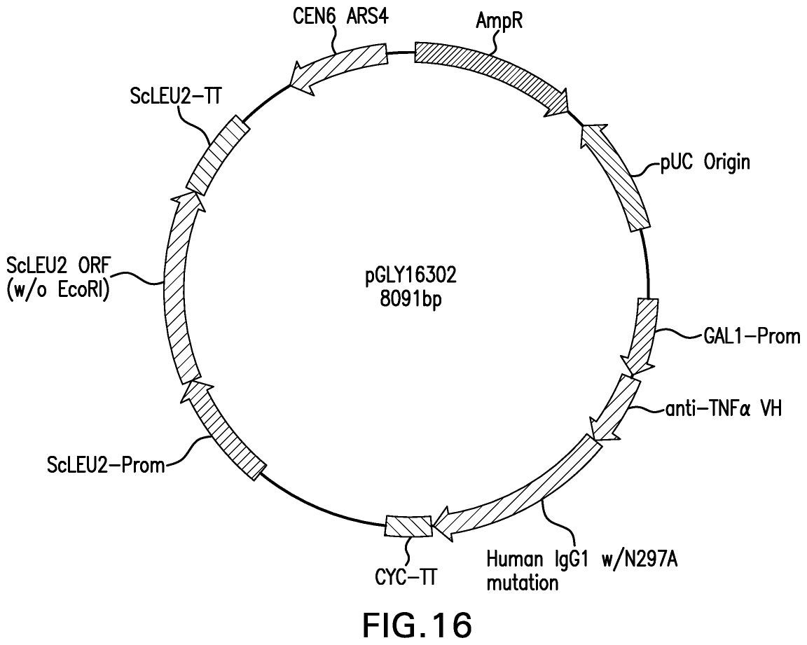

Plasmid pGLY16302 (FIG. 16) comprising a nucleic acid molecule encoding the adalimumab VH domain with a S. cerevisiae α-mating factor pre signal sequence fused to its 5′ end and an IgG1 Fc comprising the N297A mutation fused to its 3′ end (SEQ ID NO: 32) operably linked to an S. cerevisiae GAL1 promoter at the 5′ end and the CYC-TT at the 3′ end was constructed and transformed into Fc-SED1.481 expressing BJ5465 yeast cells constructed following the procedure described in Example 1. Plasmid pGLY16302 contains the S. cerevisiae LEU2 resistance gene to allow selection in media without leucine. Thus, the adalimumab HC carrying haploid yeast cells are both uracil and leucine prototrophic and tryptophan auxotrophic.

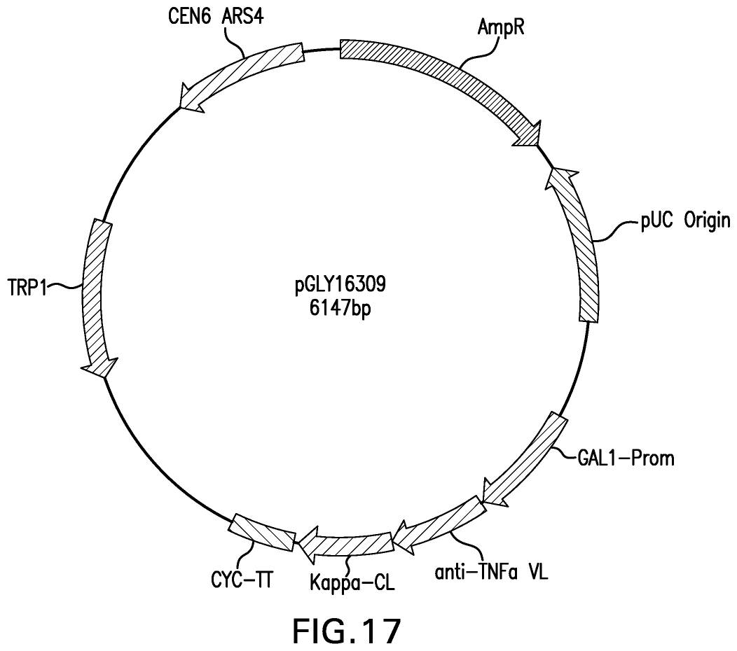

Adalimumab LC plasmid pGLY16309 (FIG. 17) comprising a nucleic acid molecule encoding the adalimumab VL domain with the S. cerevisiae α-mating factor pre signal sequence fused to its 5′ end and the human kappa LC constant domain fused to its 3′ end (SEQ ID NO: 33) operably linked to an S. cerevisiae GAL1 promoter was constructed and transformed into the isogenic parental yeast strain S. cerevisiae BJ5464 (Mating type α ura3-52 trp1 leu2Δ1 his3Δ200 pep4::HIS3 prb1Δ1.6R cant GAL, ATCC 208288). Plasmid pGLY1630) contained the S. cerevisiae TRP1 resistance gene to allow selection in media without tryptophan. The antibody LC carrying haploid yeast cells were therefore tryptophan prototrophic and uracil and leucine auxotrophic.

Anti-TNFα displaying diploid yeast were obtained by mating the adalimumab HC expressing and adalimumab LC expressing haploid yeast cells following the procedure described in Example 2 (See also FIG. 5). Detection and function of displayed the monovalent antibody fragment (H+L) was determined as described in the previous examples.

FIG. 18B shows flow cytometry analysis of Fc-SED1.481 bait mediated adalimumab monovalent antibody fragment (H+L) displayed on the cell surface. The fluorescent profiles suggested the LC expression level was high (Goat F(ab′)₂ anti-human Kappa-Alexa Fluor 647, Y-axis) and the displayed adalimumab monovalent antibody fragments (H+L) was fully functional (X-axis, NeutrAvidin conjugated to PE plus biotinylated trimeric TNFα antigen). The relatively LC intensity in FIGS. 18A and 18B were in congruence with what is known regarding expression levels of these two antibodies.

### Example 8

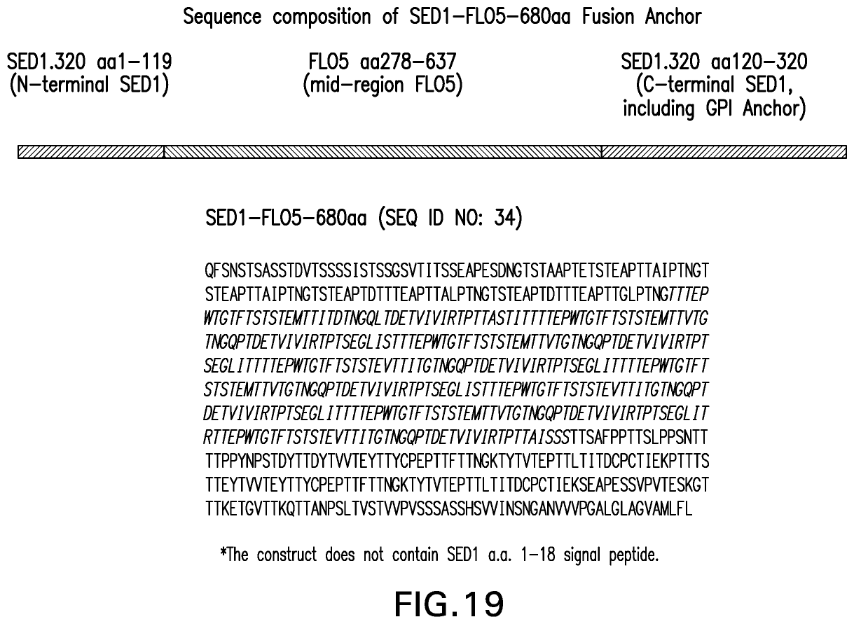

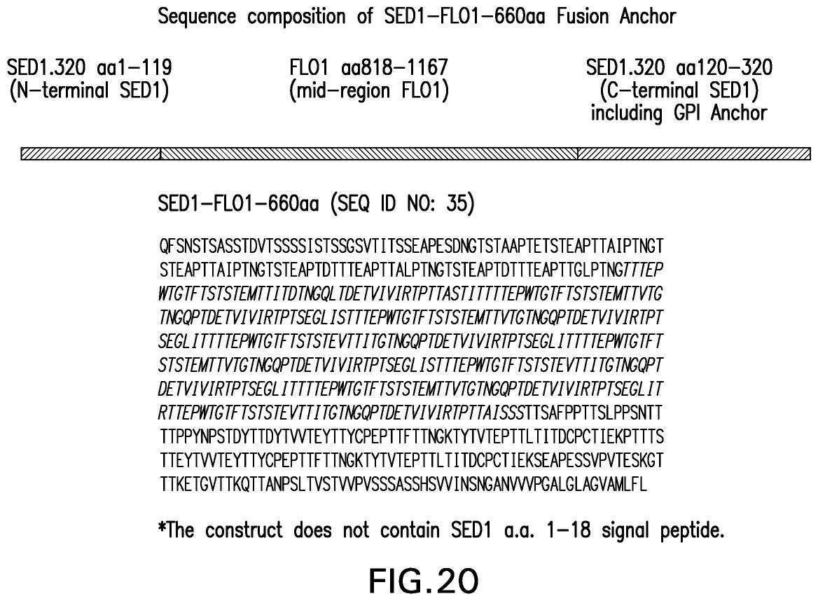

Engineering Artificial S. cerevisae SED1-FLO5 and SED1-FLO1 Fusion Protein Anchor. In addition to identifying a naturally occurring SED1.481 allele, we also engineered several SED1-based long protein anchors by creating artificially long-length SED1 fusion protein constructs. We scanned sequence databases of yeast cell wall protein sequences and focused on long yeast cell wall proteins having tandemly repeated minisatellite-like repeat sequences in the mid-region of their ORFs. An example of such a gene family that contains tandemly repeated minisatellite-like repeat sequences in the mid-region of their ORFs are members of the FLO gene family. In yeast, flocculation genes such as FLO1, FLO5, and FLO11 are long cell wall proteins carrying varied numbers of tandem repeats in the mid-region of the protein. We created two chimeric SED1.320 fusion proteins by fusing the FLO1 or FLO5 mid-region tandem repeat sequences to the mid-region of the SED1.320 protein to elongate the length of SED1.320 to 660 amino acids (SED1-FLO5.680aa fusion protein; FIG. 19) and 660 amino acids (SED1-FLO1.660aa fusion protein; FIG. 20). In both chimeric fusion proteins, the mid-region of FLO5 or FLO1 was inserted between the N- and C-terminal portions of SED1.320. This protein fusion strategy preserved the SED1 C-terminal GPI anchor sequence and SED1 N-terminal portion that is in close proximity to Fc region.

### Example 9

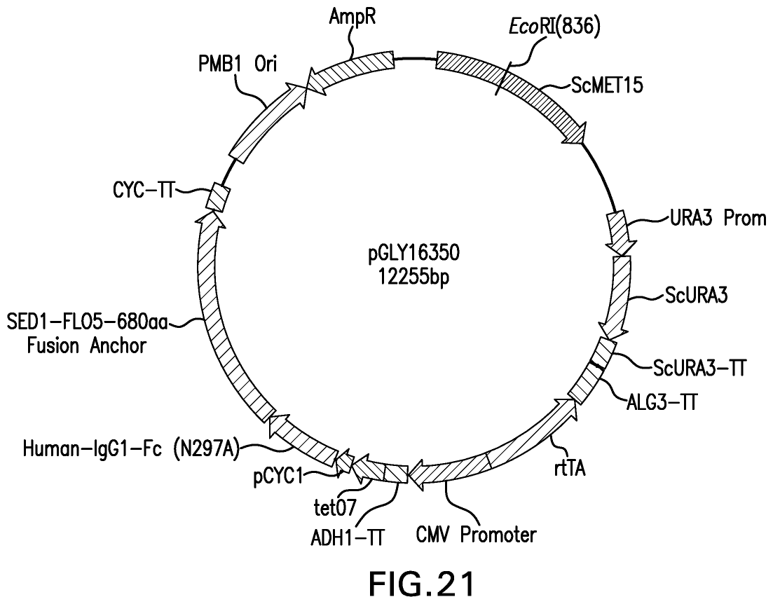

Testing Fc-SED1-FLO5.680 bait and Fc-SED1-FLO1.660 bait mediated monovalent antibody fragment (H+L) display in yeast. The Fc-SED1-FLO5.680 bait expression cassette (SEQ ID NO. 36) was constructed as follows. A codon optimized nucleic acid molecule encoding a human IgG1 Fc N297A mutein was synthesized to have at the 5′ end of the nucleic acid molecule an AfeI restriction enzyme site followed by a nucleic acid molecule encoding the S. cerevisiae α-mating factor pre signal sequence in frame with the open coding frame (ORF) for the Fc mutein and at the 3′ end of the nucleic acid molecule encoding the Fc mutein, a nucleic acid molecule encoding in-frame with the ORF encoding the Fc mutein a glycine-rich linker followed by a nucleic acid molecule encoding in-frame the SED1-FLO5.680 anchor protein and ending an FseI restriction enzyme site on the 3′ end. The Fc-SED1-FLO5.680 bait cassette was ligated to an in-house created yeast doxycycline inducible TetO7 promoter plasmid using AfeI and FseI restriction enzyme sites to generate plasmid pGLY16350 in which the expression cassette is operably linked to the TetO7 promoter (See FIG. 21). Plasmid pGLY16350 contains the S. cerevisiae MET15 gene sequence, which serves as an integration locus in the genome, and the URA3 resistance gene, to allow selection in media without uracil.

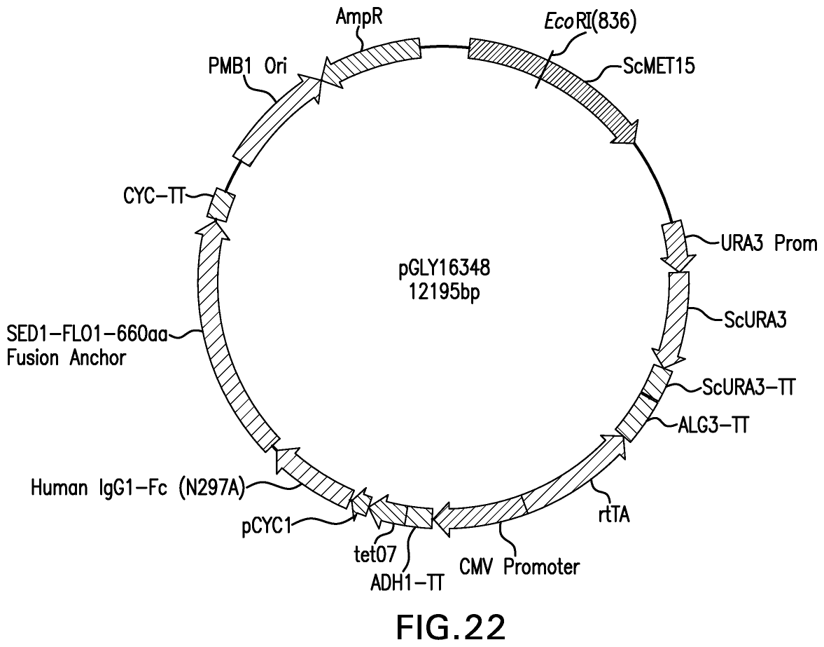

The Fc-SED1-FLO1.660 bait expression cassette (SEQ ID NO: 37) was constructed as follows. A codon optimized nucleic acid molecule encoding a human IgG1 Fc N297A mutein was synthesized to have at the 5′ end of the nucleic acid molecule an AfeI restriction enzyme site followed by a nucleic acid molecule encoding the S. cerevisiae α-mating factor pre signal sequence in frame with the open coding frame (ORF) for the Fc mutein and at the 3′ end of the nucleic acid molecule encoding the Fc mutein, a nucleic acid molecule encoding in-frame with the ORF encoding the Fc mutein a glycine-rich linker followed by a nucleic acid molecule encoding in-frame the SED1-FLO1.660 anchor protein and ending an FseI restriction enzyme site on the 3′ end. The Fc-SED1-FLO1.660 bait cassette was ligated to an in-house created yeast doxycycline inducible TetO7 promoter plasmid using AfeI and FseI restriction enzyme sites to generate plasmid pGLY16348 in which the expression cassette is operably linked to the TetO7 promoter (See FIG. 22). Plasmid pGLY16348 contains the S. cerevisiae MET15 gene sequence, which serves as an integration locus in the genome, and the URA3 resistance gene, to allow selection in media without uracil.

Plasmids pGLY16350 and pGLY16348 were each separately transformed into the BJ5465 strain to generate Fc-SED1-FLO5.680 bait and Fc-SED1-FLO1.660 bait expressing BJ5465 strains, respectively. The anti-HER2 HC-expressing plasmid pGLY15562 was transformed into each BJ5465 yeast strain. The anti-HER2 LC-expressing plasmid pGLY16304 was transformed into the isogenic parental yeast strain S. cerevisiae BJ5464. Anti-HER2 displaying diploid yeast were obtained by mating an HC-expressing and a LC-expressing haploid yeast cell following the procedure in Example 2 (See FIG. 5). Detection and function of displayed the monovalent antibody fragment (H+L) was determined as described in the previous examples.

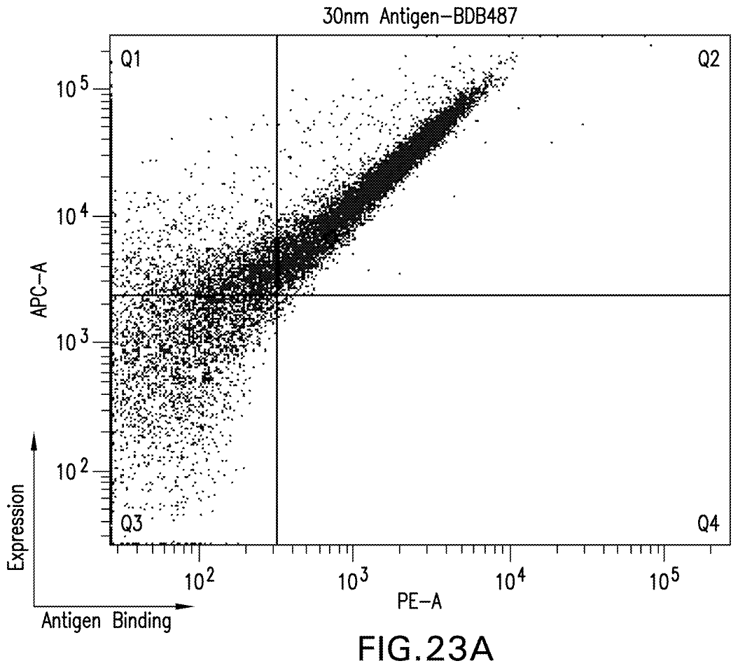

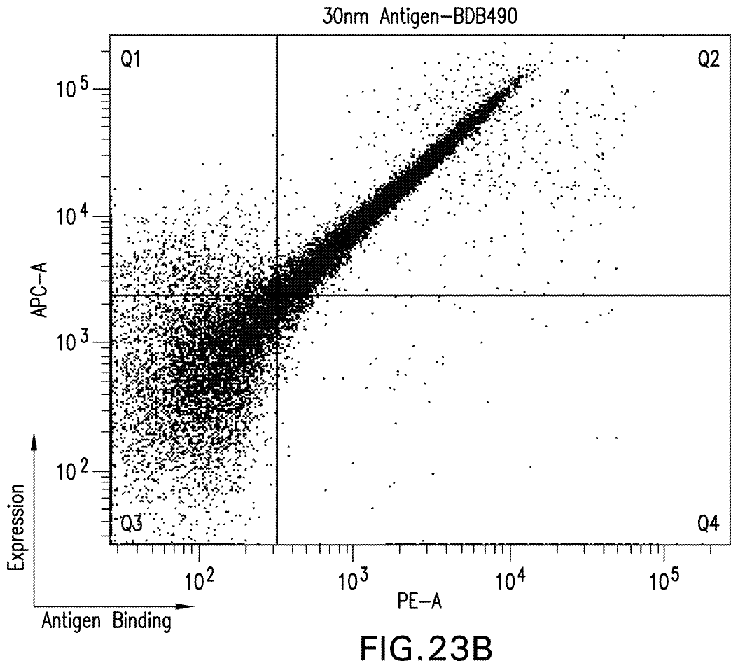

FIG. 23A and FIG. 23B shows flow cytometry analysis of SED1-FLO5.680 and SED1-FLO1.660 bait mediated anti-Her2 monovalent antibody fragment (H+L) display on the cell surface. The fluorescent profiles suggested the LC expression level was high (Goat F(ab′)₂ anti-human Kappa-Alexa Fluor 647, Y-axis) and the displayed anti-HER2 monovalent antibody fragment (H+L) were fully functional (X-axis, NeutrAvidin conjugated to PE plus biotinylated human HER2 Ectodomain antigen).

These results demonstrate that efficient monovalent antibody fragment (H+L) display in S. cerevisiae surface may be achieved by increasing the length of SED1 protein anchor beyond its native 320 amino acids. Long-length SED1 anchor proteins may be obtained from naturally occurring SED1 alleles or by constructing artificially elongated SED1 proteins.

### Example 10

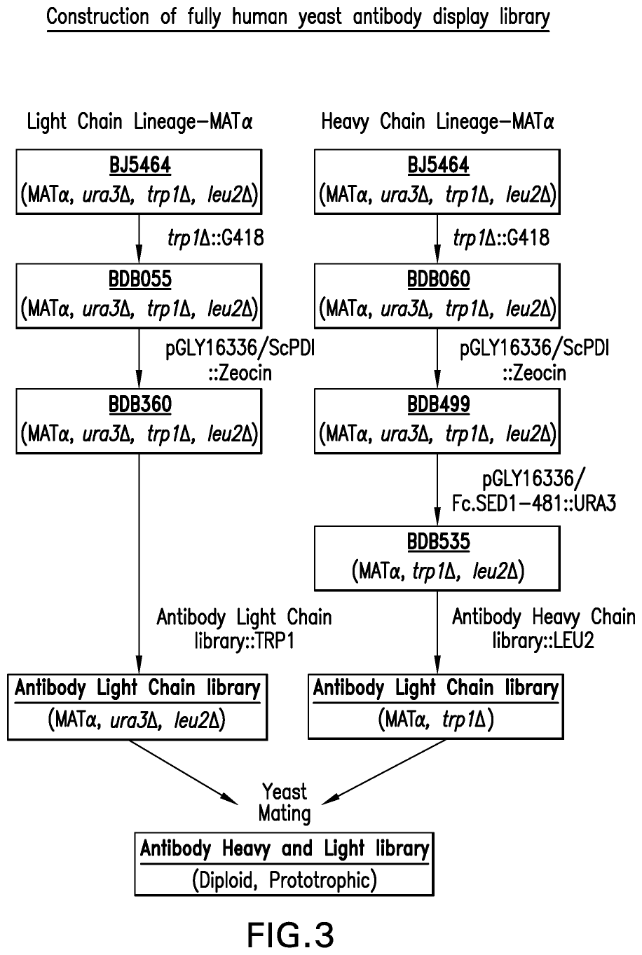

Engineering yeast host strains BDB360 (MATα) and BDB535 (MATα) for combinational yeast display (HC×LC) antibody mating library. To create yeast host strains for construction of combinational yeast display (HC×LC) antibody mating library, we started from parental yeast strains BJ5464 and BJ5465 and engineered complete TRP1 gene knockouts. Yeast strains BJ5464 and BJ5465 carry an amber non-sense mutation within TRP1/YDR007W ORF. The reversion of the amber mutant would slowly cause loss of library's TRP sensitivity: knocking out the entire TRP1 ORF eliminated the possibility of amber reversion. Construction of the strains and their use are illustrated in FIG. 3.

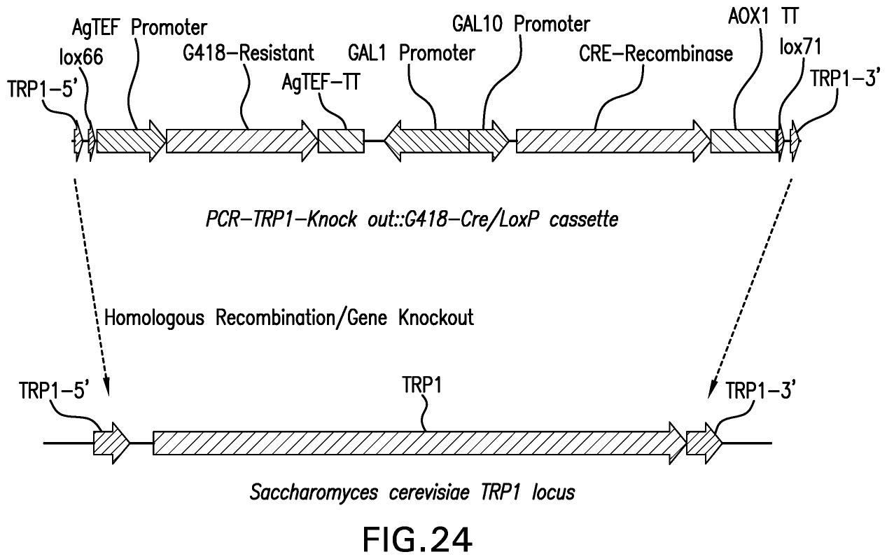

FIG. 24 shows a linear Cre-LoxP G418 resistant DNA construct that targets S. cerevisiae TRP1 locus. The DNA fragment was assembled using DNA synthesis plus PCR assembly. To generate the Cre-LoxP TRP1 knock-out DNA replacement allele, 10 μg linear PCR product was transformed into BJ5464 and BJ5465 yeast strains and the transformants were selected at 30° C. on YPD plates containing 50-200 μg/mL G418 antibiotic. To confirm complete knockout of TRP1, the candidate knockout strains were inoculated in SD-TRP media and no growth was observed. Isolated genomic DNA was used as the basis for PCR assays confirming the presence of the properly sized insert and the kanamycin resistance marker. Later, a confirmed TRP1 knockout clone was cultivated in Yeast Extract-Peptone-Galactose medium (1% Yeast Extract, 2% Peptone, 2% Galactose) in a 50 mL shake flask overnight, to induce expression of the GAL10 promoter-driven Cre-LoxP recombination. This Cre-LoxP recombination recycled the G418 marker and the strains became G418 sensitive.

Strain BDB055 (MATα) and BDB066 (MATα), which were derived from BJ5464 (MATα) and BJ6455 (MATα), respectively, exhibited the proper genomic and phenotypic characteristics. The two strains were used for the subsequent experiments.

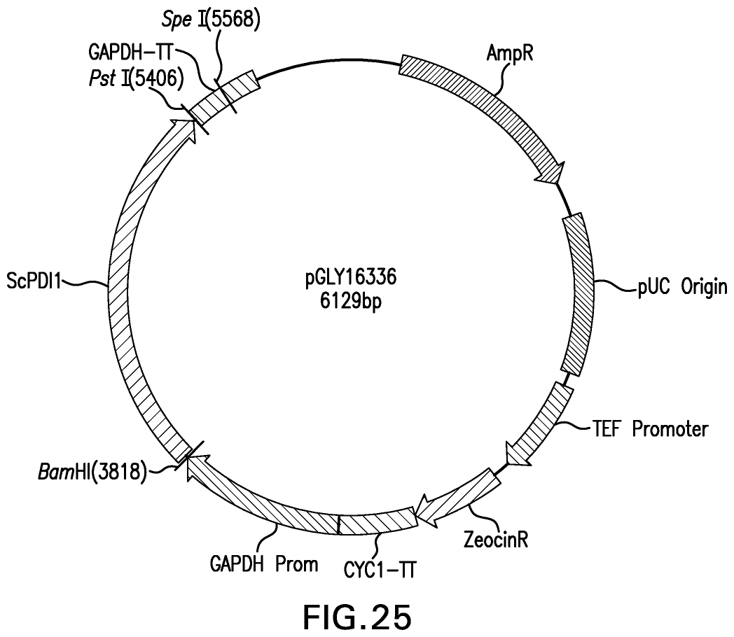

FIG. 25 shows the map of pGLY16336. Plasmid pGLY16336 is a yeast integrating vector carrying a S. cerevisiae PDI1 gene over-expression cassette operably linked to the constitutive glyceraldehyde-3-phosphate dehydrogenase (GAPDH) promoter. Yeast PDI1 encodes is a protein disulfide isomerase having the amino acid sequence set forth in SEQ ID NO. 42. It has been shown that overexpression of yeast PDI increases secretion of recombinant proteins produced in S. cerevisiae. To generate PDI over-expressing yeast strains, plasmids pGLY16336 was linearized using SpeI restriction enzyme to facilitate the plasmid's integration to yeast GAPDH terminator locus. Then. 10 μg linearized vector was transformed to yeast strain BDB055 (MATα) and BDB066 (MATa), respectively, to create yeast strains BDB360 (MATα) and BDB499 (MATa). These two strains were then used for the subsequent experiments.

In this example, Fc-SED1.481 was selected as the anchor protein to build the fully human (H+L) display library. Plasmid pGLY16356 was linearized using EcoRI restriction enzyme to facilitate the plasmid's integration to yeast MET15 locus and then transformed into yeast strain BDB499 to generate Fc-SED1.481 bait strain BDB535 (MATa). Yeast strain BDB360 (MATα) and BDB535 (MATa) were used as the host strains for the combinational yeast display (HC×LC) antibody mating library construction.

### Example 11

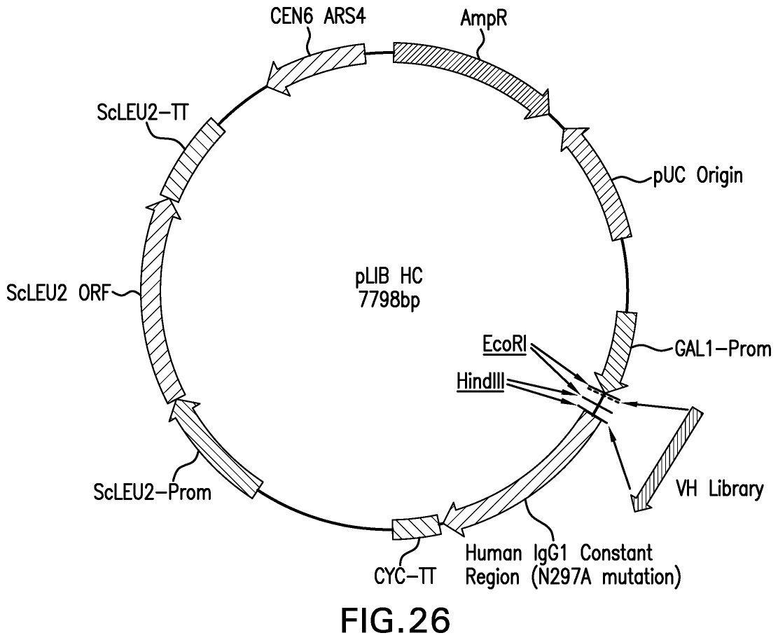

Highly efficient yeast DNA transformation for the very large size (>10⁹) antibody HC and LC yeast haploid libraries. FIG. 26 shows the map of heavy chain library plasmid pLIB-HC. Plasmid pLIB-HC is a yeast centromere plasmid that maintains a single copy number in a yeast cell. Plasmid pLIB-HC is used to build the antibody HC haploid yeast library, pLIB-HC carries a yeast LEU2 selection marker and HC expression is under the control by the GAL1 promoter.

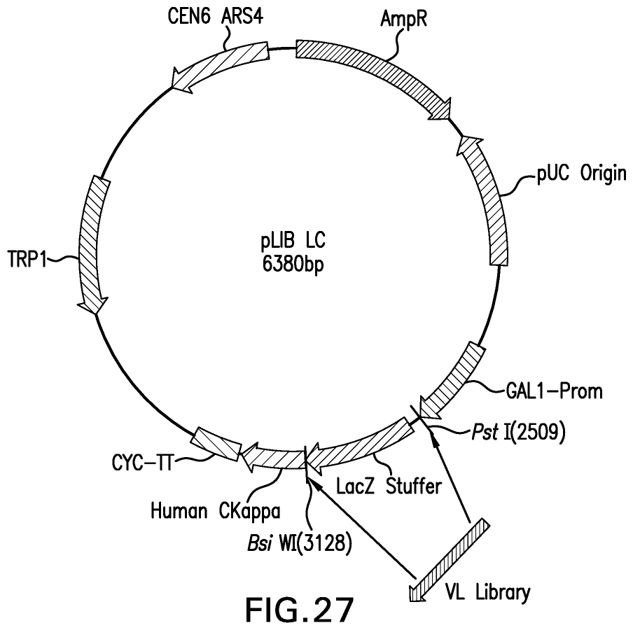

FIG. 27 shows the map of light chain library pLIB-LC. Plasmid pLIB-LC is a yeast centromere plasmid that maintains a single copy number in a yeast cell. Plasmid pLIB-LC is used to build the antibody LC haploid yeast library. Plasmid pLIB-LC carries a yeast TRP1 selection marker and LC expression is under the control by the GAL1 promoter.

We developed a high efficiency yeast transformation protocol that enabled the construction of very large size (greater 10⁹) yeast antibody HC and LC haploid cell libraries. Yeast strains BDB360 (MATα) or BDB535 (MATa) was inoculated from a frozen yeast stock vial to grow in one-liter YPD media in a two-liter baffled shake flask at 30° C., 220 rpm overnight. The number of yeast cells stored in the frozen vial was fine-calculated so that the cell culture density reached 1.6 OD (at 600 nm) after 16 hours growth in YPD media.

The next day, the entire one liter of cell culture was centrifuged at 3,600×g for three minutes to produce a cell pellet. The cell pellet was washed three times in 500 mL ice-cold “1 M sorbitol+1 mM CaCl₂” buffer and then resuspended in 100 mL “0.1 M LiAc+2.5 mM TCEP” pre-treatment buffer and incubated with shaking for 30 minutes at 30° C., at 120 rpm. Next, the cells were pelleted at 3,600×g for three minutes at 4° C., and the cell pellet was washed two times with ice-cold “1 M sorbitol+1 mM CaCl₂” buffer. Cells were then resuspended in ice-cold “1 M sorbitol+1 mM CaCl₂” buffer to a final concentration of 2×10⁹ cells/mL.

Heavy chain vector pLIB-HC was double digested with EcoRI and HndIII restriction enzymes and the vector fragment was purified by agarose gel electrophoresis. Light chain vector pLIB-LC was double digested with PstI and BsiWI restriction enzymes and the vector fragment was purified by agarose gel electrophoresis. Four μg of restriction enzyme digested pLIB-HC and pLIB-LC plasmids were each mixed with 12 μg PCR amplified antibody VH nucleic acid library or antibody VL nucleic acid library, respectively, and each mixture was added to 400 μL of yeast competent cells in an electroporation cuvette. The antibody VH nucleic acid library was mixed with yeast strain BDB535(MATα) and the antibody VL nucleic acid library was mixed with yeast strain BDB360 (MATα). The PCR amplified VH and VL libraries carry about 400-600 bp sequences on both the 5′ and 3′ends that overlap with the sequences at the ends of the enzyme digested vector. The VH and VL library fragments recombine with linearized vectors and form circular HC and LC expression plasmids by homologous recombination inside Saccharomyces cerevisiae yeast cells.

Electroporation of the mixtures was then conducted with a Biorad Gene Pulser Xcell electroporation system using the exponential decay protocol with a two mm electroporation cuvette under the following parameters: 2.6 kV, 200Ω resistance, 25 μF capacitance, typically resulting in a time constant of 3.9-4.2 millisecond. After electroporation, recovery media (equal parts YPD media and 1 M sorbitol) was added to the yeast cells and yeast cells were incubated with shaking at 120 rpm for one hour at 30° C. The Yeast cells were then pelleted at 3,600×g for three minutes and the yeast cell pellet resuspended in 1 M sorbitol at dilutions of 10⁻⁶, 10⁻⁷, and 10⁻⁸, and plated on appropriate complete minimal (CM) glucose dropout plate for calculating the library size.

The antibody VH nucleic acid library transformed into yeast strain BDB535 (MATa) produced the antibody HC haploid yeast library, which can be selected and propagated in CM glucose minus leucine dropout media, and the antibody VL nucleic acid library transformed into yeast strain BDB360 (MATα) produced the antibody LC haploid yeast library, which can be selected and propagated in CM glucose minus tryptophan dropout media.

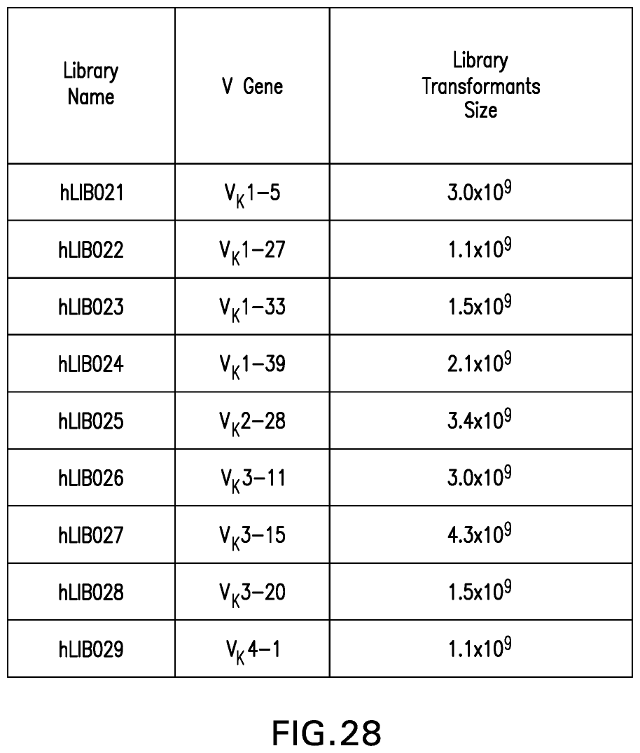

In this example, a total of nine separate antibody LC nucleic acid libraries were individually transformed into yeast strain BDB360 (MATα) following the above protocol. FIG. 28 lists the VL genes and library size of each antibody LC haploid yeast library. For every library transformation, four to ten electroporation cuvettes were used, and the size of all LC libraries were greater than 10⁹.

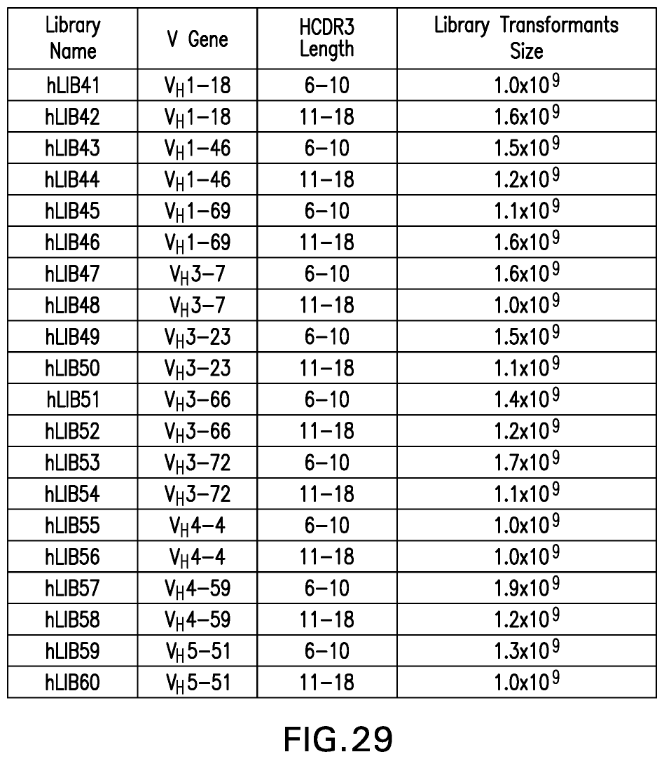

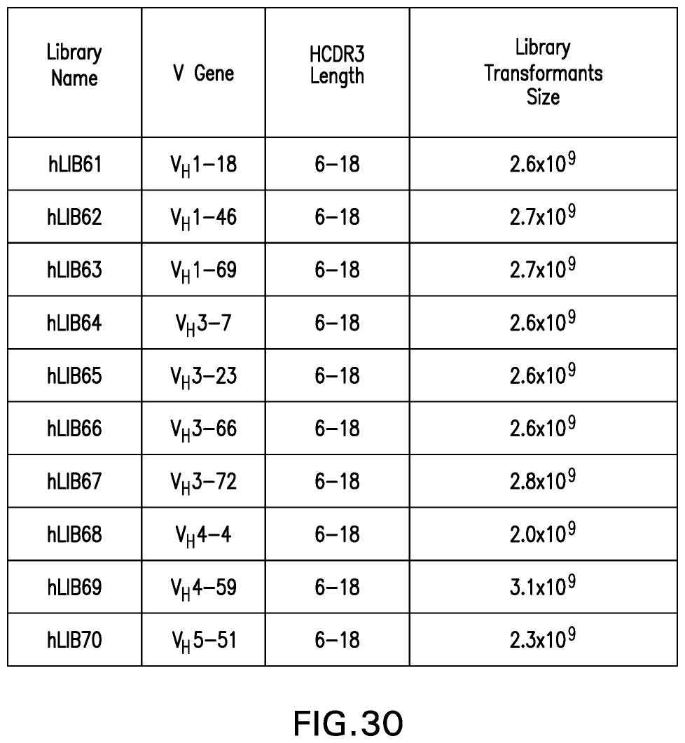

Twenty antibody HC nucleic acid libraries were individually transformed into yeast strain BDB535 (MATa). FIG. 29 and FIG. 30 lists the V genes, HCDR3 length, and the library size of each antibody HC haploid yeast library. There are 10 VH genes. For every library transformation, four to ten electroporation cuvettes were used, and the size of all heavy chain libraries were greater than 10⁹.

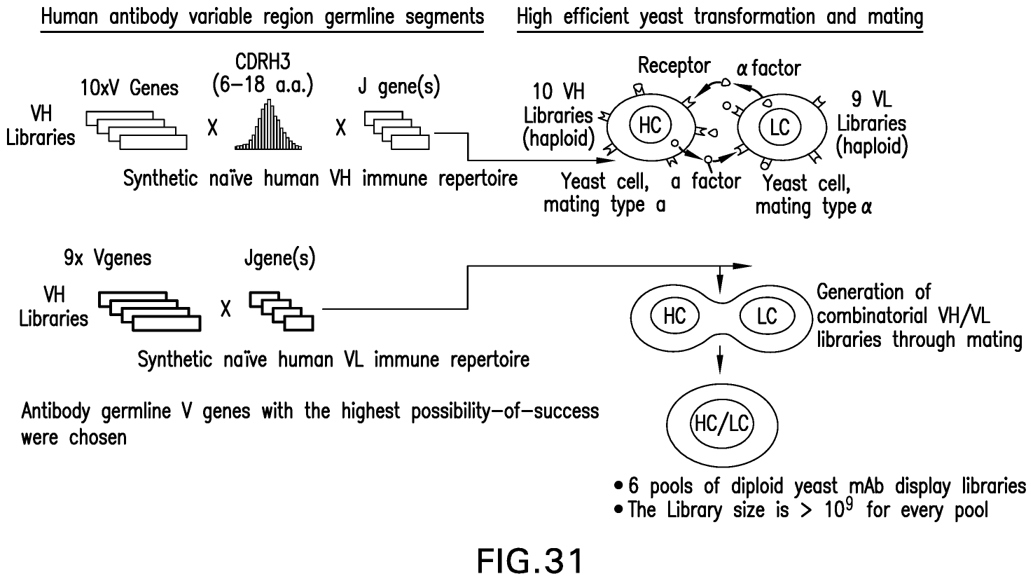

Two antibody HC nucleic acid libraries from the same VH gene(s) with different HCDR3 length distributions (6-10 and 11-18 amino acids) were mixed together in a 1:1 ratio to create a combined VH gene antibody HC haploid yeast library (FIG. 31). The 1:1 mixing ratio was selected in order to extensively cover shorter length HCDR3 (6-10 amino acids) diversity.

### Example 12

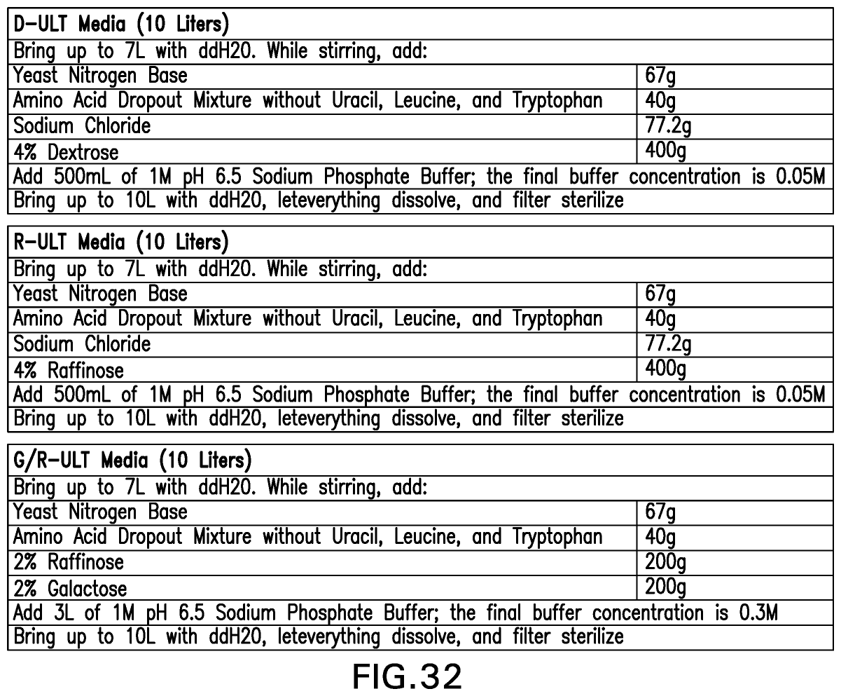

Highly efficient yeast mating was used to create very large size (greater than 10⁹) combinatorial HC+LC diploid display libraries. We developed a large-scale yeast mating protocol, which enabled the construction of very large size (greater than 10⁹) yeast HC+LC combinatorial mating libraries. Dextrose phosphate amino acid drop out media (FIG. 32) was used for selection and growth of yeast display libraries. Antibody HC and LC haploid yeast libraries were refreshed and grown overnight in their respective selective media (D-UL for antibody HC haploid yeast libraries, D-T for antibody LC haploid yeast libraries) in ten-fold excess of library size. To make sure library cells were in early-to-mid log phase when harvested at the next day, the starting cell density was 0.5 OD/mL at 600 nm. Cells were grown at 24° C. for 16 hours at 220 rpm.

Sixteen hours later, after cell density was measured, 3×10⁹ antibody HC haploid yeast library cells and 3×10⁹ antibody HC haploid yeast library cells were mixed in a 500 mL conical tube and centrifuged at 3,600×g for three minutes. The supernatant fraction was decanted, and the cell pellet resuspended in 20 milliliters YPD media. Two mL of cell suspension (or 3×10⁸ cells per haploid library) was plated onto one 150 mm diameter YPD petri dish so that the cell density is 1.7×10⁷ cells/cm². 10 YPD plates were used for one mating library construction to achieve greater than 10⁹ library size. An L-shaped cell spreader was used to spread the cells homogenously on the plate. The plates were incubated at 30° C. for six hours.

In the afternoon, the plates were taken out and 25 mL of water was added to each plate, and an L-shaped spreader was used to scraped down cells. Cells were transferred to a 250 mL conical tube and pelleted at 3,600×g for five minutes. The cells were washed once in 250 mL deionized water, pelleted at 3,600×g for five minutes, and the cell pellet resuspended to grow in one liter of D-ULT selective media to select diploid cells in a two L flask.

To estimate mating efficiency, 10 μL of cells were taken from the one liter cell suspension and diluted to plate onto 10⁻⁶, 10⁻⁷, and 10⁻⁸ serial dilution plates. Diploid cells were selected to grow on CM Glucose minus uracil, leucine, tryptophan plate and incubated at 30° C. for three days.

Ten antibody HC haploid yeast libraries and nine LC haploid yeast libraries were mated according to the above protocol to create 90 antibody HC and LC combinatorial diploid (H+L diploid) display libraries. Each H+L diploid library had a greater than 10⁹ library size. The 1.0-1.5×10⁹ H+L diploid display library size corresponds to a typical 35%-50% mating efficiency.

### Example 13

Growth and induction of antibody H+L diploid display libraries. FIG. 32 lists the media used for growth, de-repression, and induction of the yeast display libraries. The antibody heavy chain+light chain diploid mating libraries were grown and propagated in D-ULT media. To induce antibody surface display, an appropriate amount of yeast library cells, which cover 10× library size were refreshed to grow in D-ULT media, at a cell density of 0.5 OD (at 600 nm) per milliliter. Libraries were grown for 24 hours at 220 rpm at 30° C. In the presence of dextrose, repressor proteins bind to the negative regulatory sites in the GAL1 promoter and represses transcription of a Gal4p transcriptional activator bound to positive regulatory sites in the GAL1 promoter but the Gal4p is inactive because it is bound to the repressor protein, Gal80p. In the absence of doxycycline, the TetO7 promoter is turned off. Thus, under said conditions there is no expression of the Fc-SED1.481 bait and no or trace expression of the antibody heavy or light chains.

Twenty-four hours later, an appropriate amount of yeast library cells (covering 10× library size) was centrifuged at 3,600×g for three minutes to pellet the antibody H+L diploid display library cells. The antibody H+L diploid display library cells were then resuspended in R-ULT media at a cell density of 0.5 OD per milliliter for de-repressing GAL1 promoter and in the presence of 10 μg/mL doxycycline to induce expression the Fc-SED1.481 bait. The raffinose de-represses the GAL1 promoter but does not remove the Gal80p repressor protein bound to Galp4. Thus, there is expression of the Fc-SED1.481 bait and some low level transcription from the GAL1 promoter. The resuspended cells were grown overnight for 16 hours at 220 rpm at 30° C.

The next morning, an appropriate amount of antibody H+L diploid display library cells (covering 10× library size) were centrifuged at 3,60W g for three minutes and the antibody H+L diploid display library cell pellet was resuspended in G/R-ULT media at a cell density of 1.0 OD per milliliter to induce the expression of the antibody heavy and light chains and in the presence of 10 μg/mL doxycycline to induce expression the Fc-SED1.481 bait. The galactose removes the Gal80p repressor, which induces high level (˜1000 fold the level observed in the presence of dextrose) transcription from the GAL1 promoter. The antibody H+L diploid display libraries were induced for 24 hours, at 220 rpm, at 24° C.

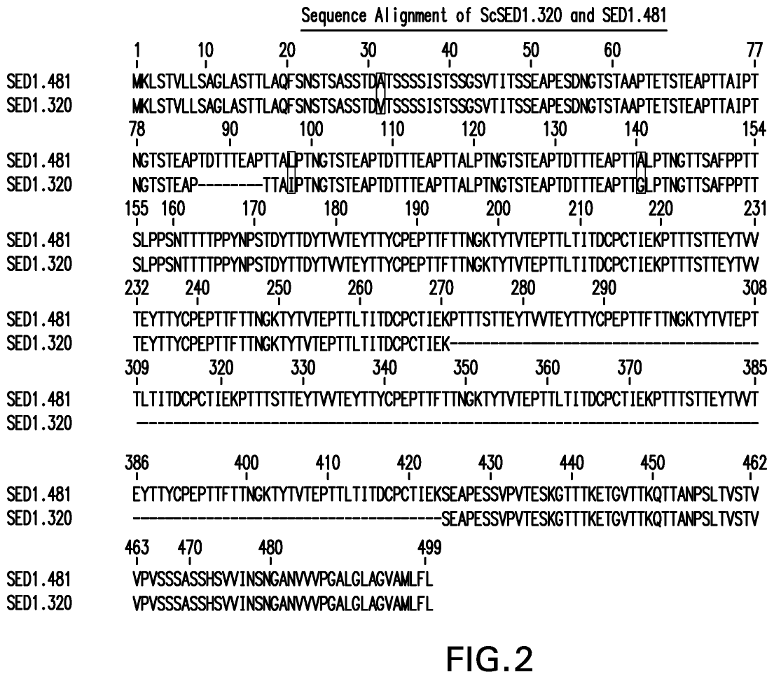

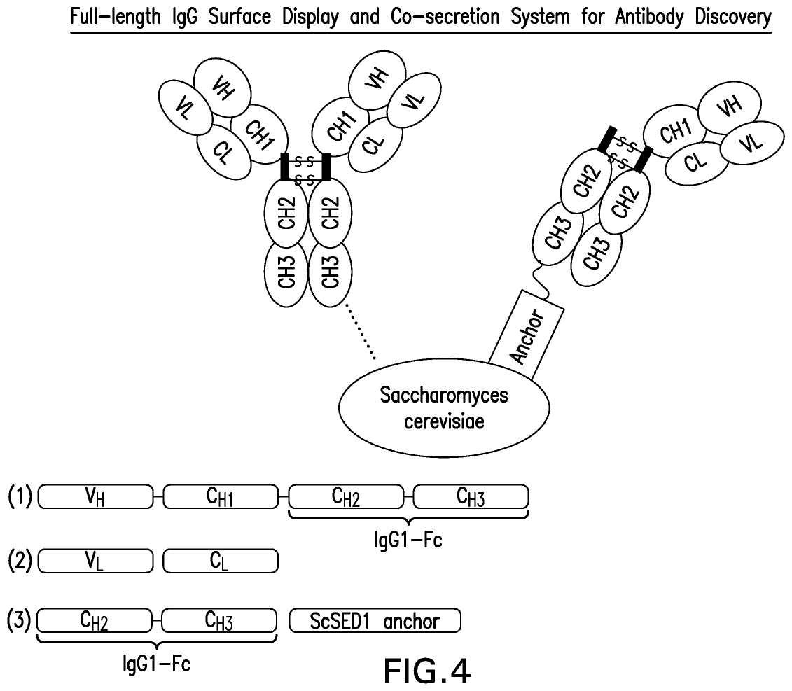

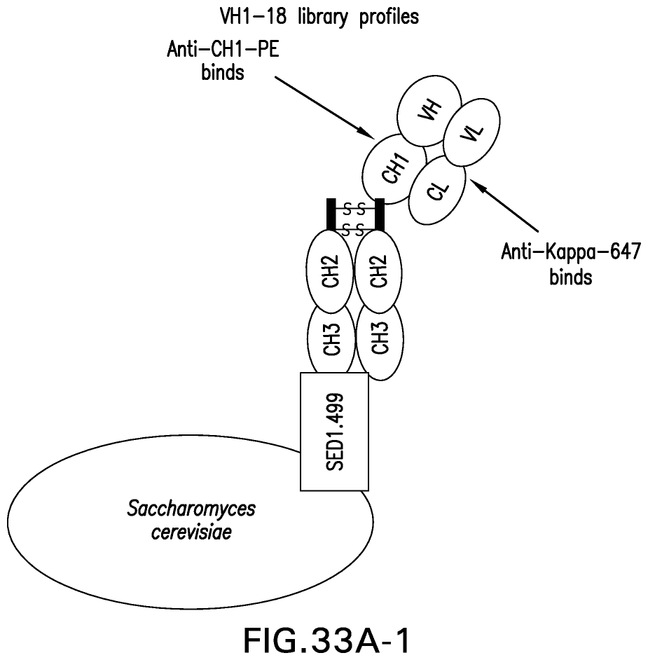

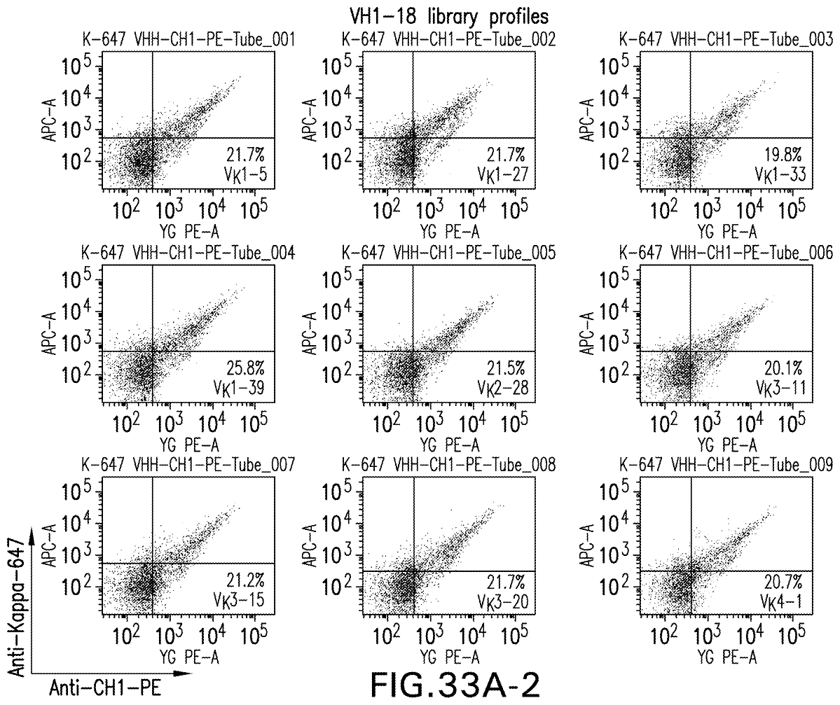

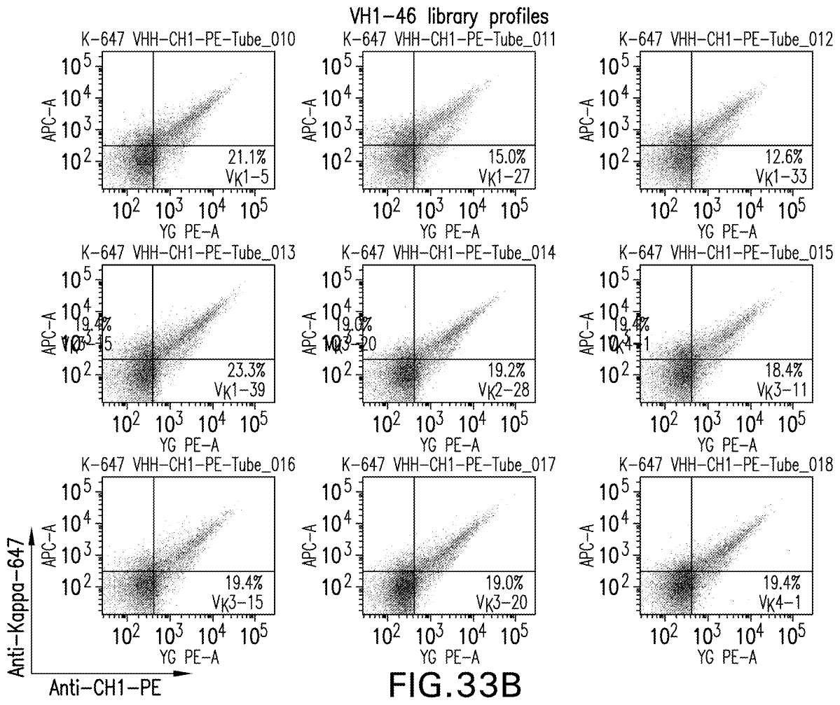

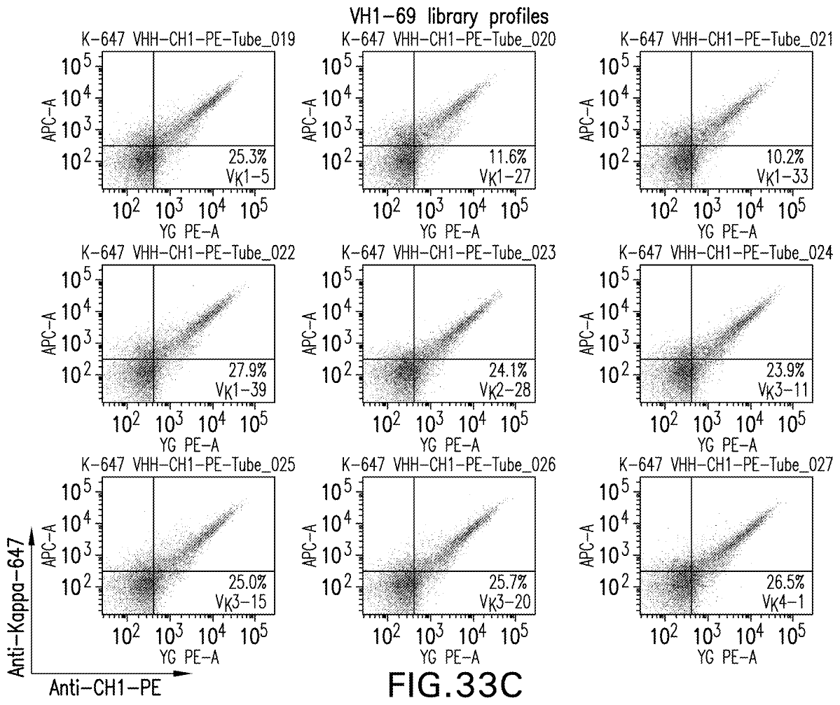

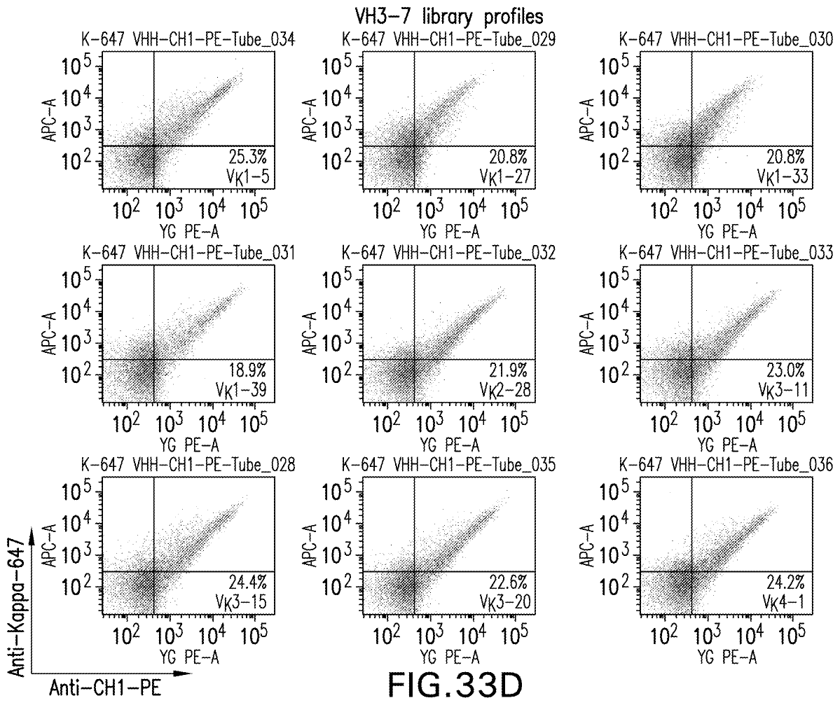

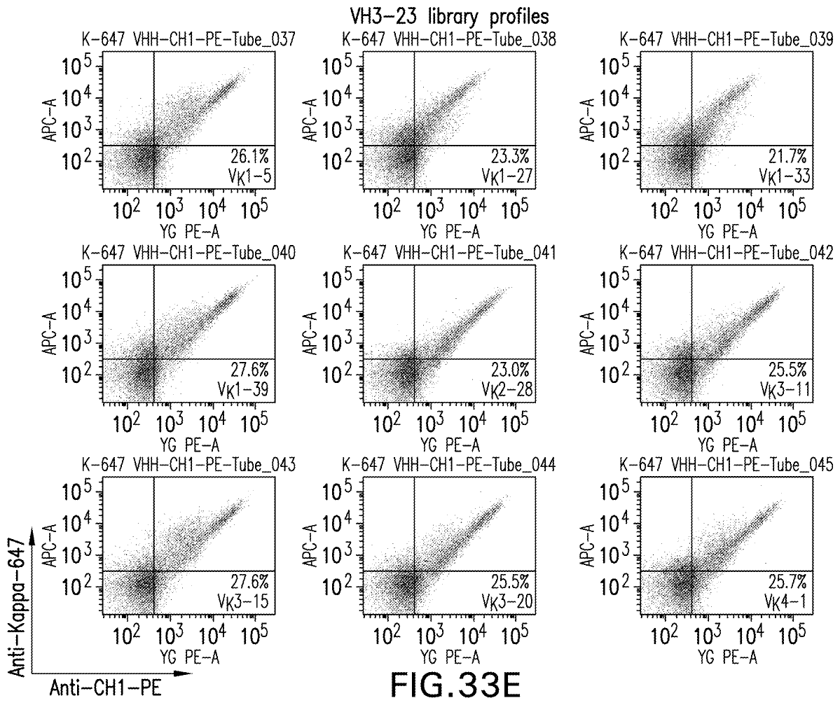

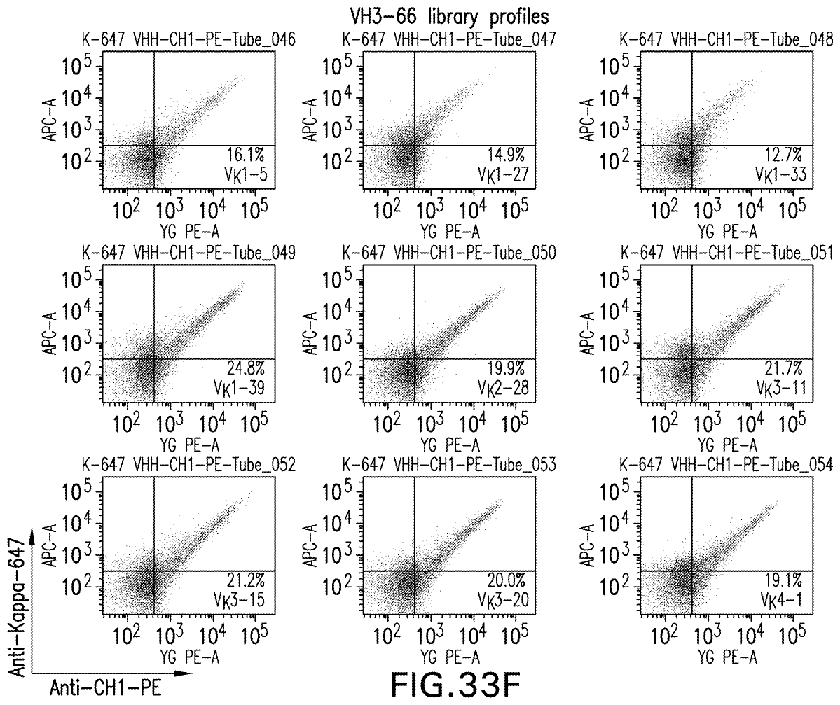

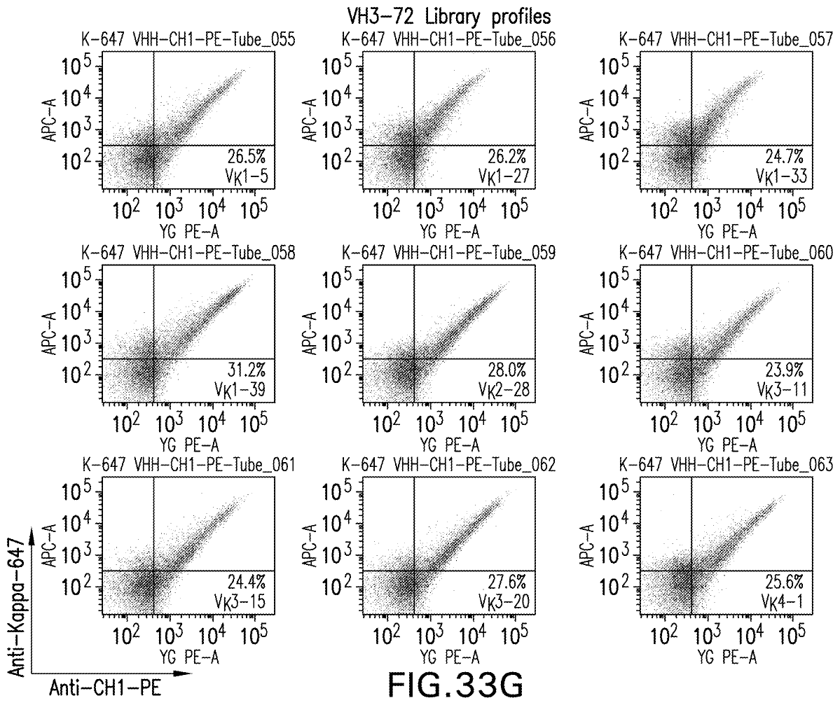

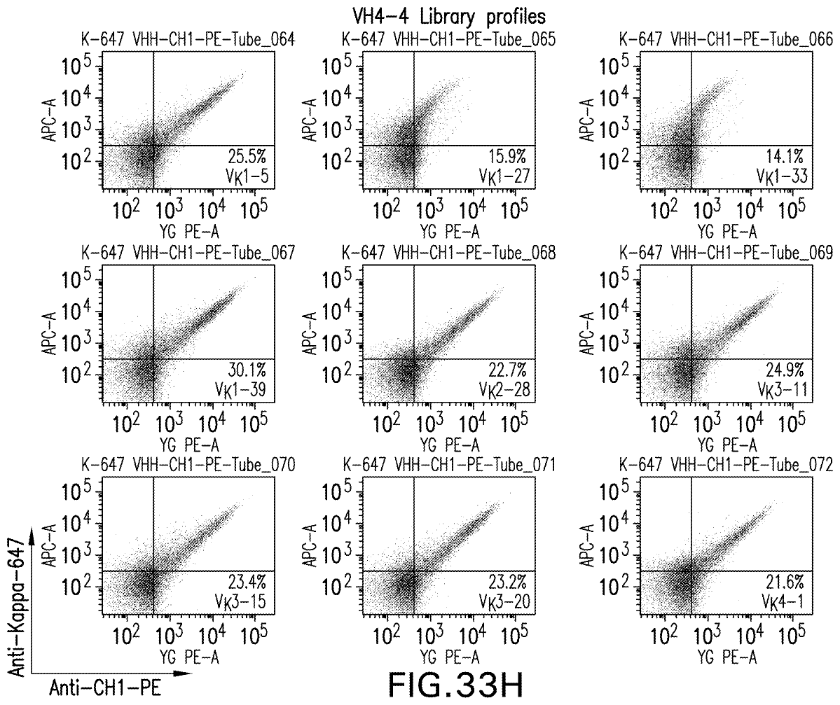

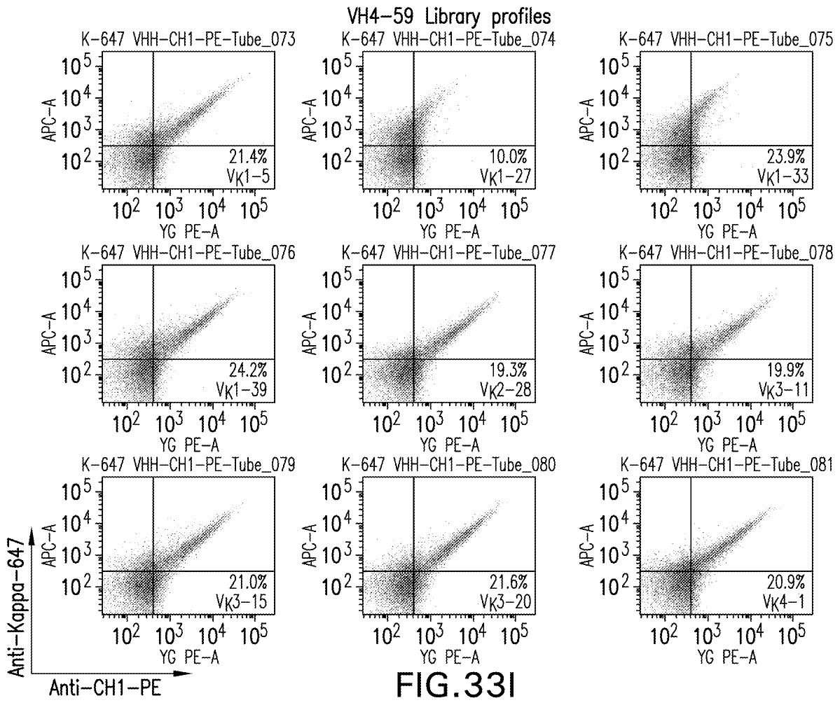

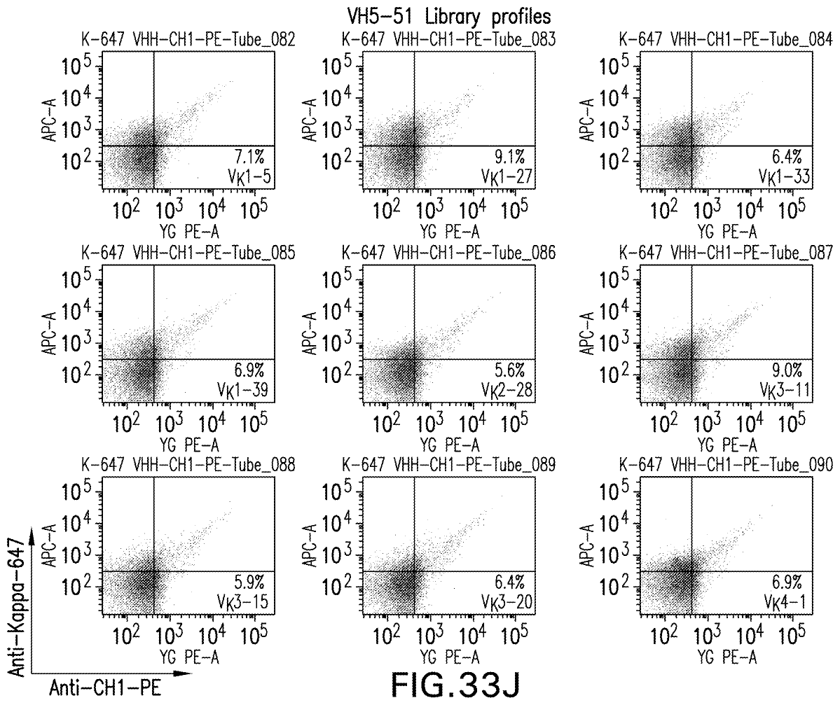

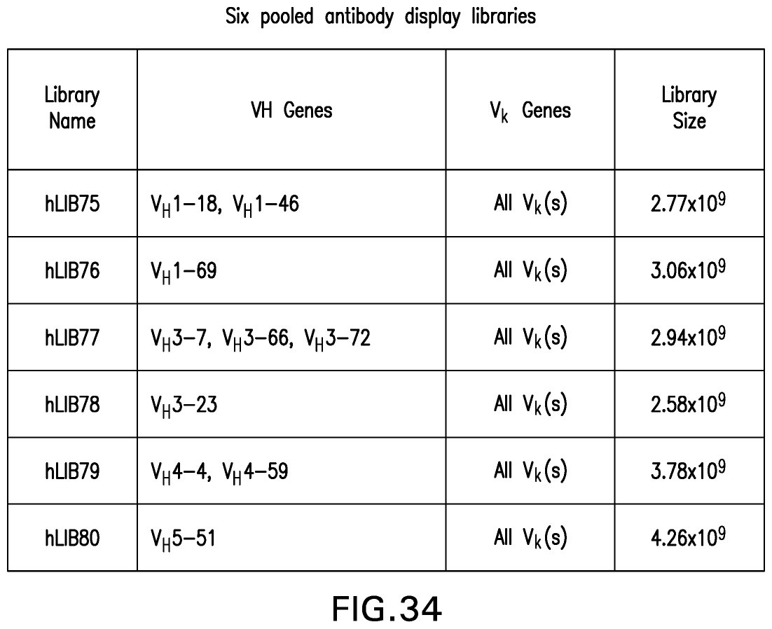

FIG. 33A-1 through FIG. 33J show the flow cytometric expressional analyses of ten different HC haploid yeast libraries with each of the nine LC haploid yeast libraries. Goat F(ab′)₂ anti-human Kappa-Alexa Fluor 647 was used to detect LC expression (Y-axis) and biotinylated llama V_{HH} anti-human CH1 (ThermoFisher), then and reacted with NeutrAvidin-R-phycoerythrin (R-PE) conjugated (X-axis). The results of FIG. 33A-1 through FIG. 33J suggest that the expression profiles were predominantly determined by the specific VH-containing library being used. VL-containing libraries played a minor role on determining antibody assemble and expression.

To further reduce the number of libraries so that one scientist can perform a round of naïve selection in a day, 90 HC×LC diploid libraries—were mixed together to create six merged libraries based on their relative expression levels. FIG. 34 shows the VH/VL genes and library size of the six merged human naïve yeast diploid libraries.

### Example 14

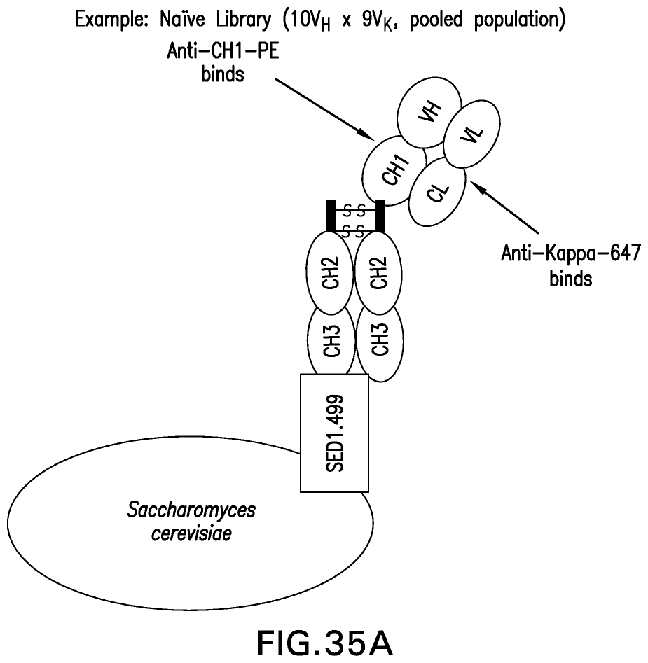

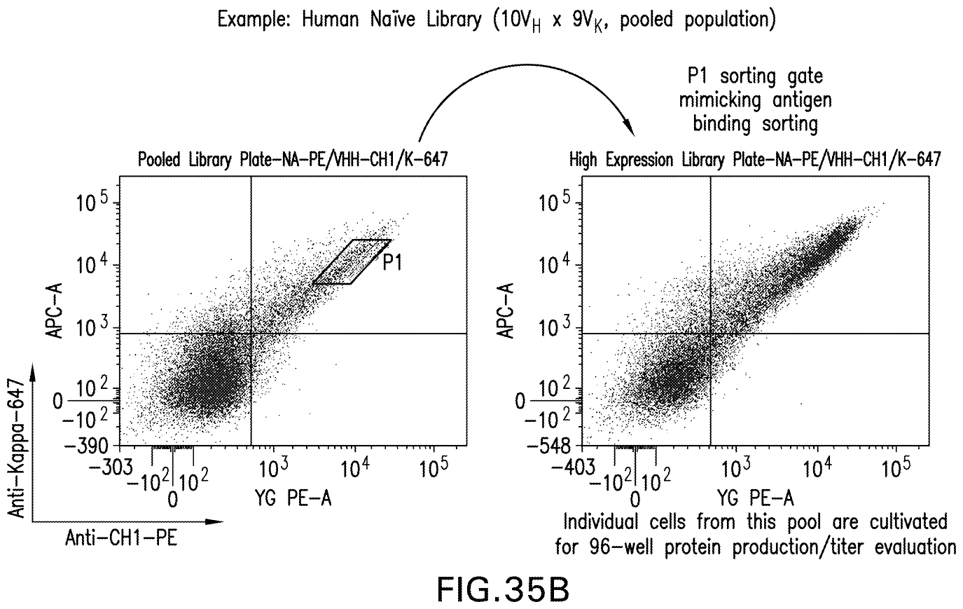

Fluorescence activated cell sorting of yeast libraries and yeast antibody secretion. We mixed all six antibody H+L diploid display libraries in equal cell number ratio, induced antibody display on the cell surface, and executed one round of fluorescence-activated cell sorting of the yeast library cells based on the Kappa and CH1 signals (FIG. 353). The P1 quadrilateral sorting gate was drawn and used as a mockup to mimic typical antigen binding events in typically yeast display antibody discovery procedure. FIG. 35B left panel shows data relating to library cells before sorting, and the right panel shows data relating to cells containing the library after sorted once. The data indicated that sorting resulted in significant enrichment on antibody expression in the desired quadrant after one round of sorting.

Ninety-six randomly selected yeast clones from FIG. 35B library-sorted output were cultivated in a 96-well format for full-length antibody production. Briefly, single yeast colonies from the antibody H+L diploid display library-sorted output were inoculated to grow in 1,000 μL volume D-ULT media in the 96-deep well plate format. The cells were grown in 30° C. at 850 rpm overnight.

The next day, 300 μL of yeast culture were mixed with 700 μL of fresh D-ULT media to refresh to grow. The cells were grown in 30° C. at 850 rpm overnight. On the next day, cells were pelleted and the cell pellet resuspend in the induction media with a protein-O-mannosyltransferase (PMT) inhibitor. The induction medium comprises 200 mM Sodium Phosphate pH 6.5, 1.34% YNB (yeast nitrogen base), 4% Peptone, 2% Yeast Extract, 1% Dextrose, 4% Galactose, and 3.6 μg/mL PMT inhibitor (see U.S. Pat. No. 8,309,325). The cells were induced at 30° C. at 850 rpm overnight. Cells were feed with the feed media comprising 200 mM Sodium Phosphate pH 6.5, 1.34% YNB, and 20% Galactose and grown overnight.

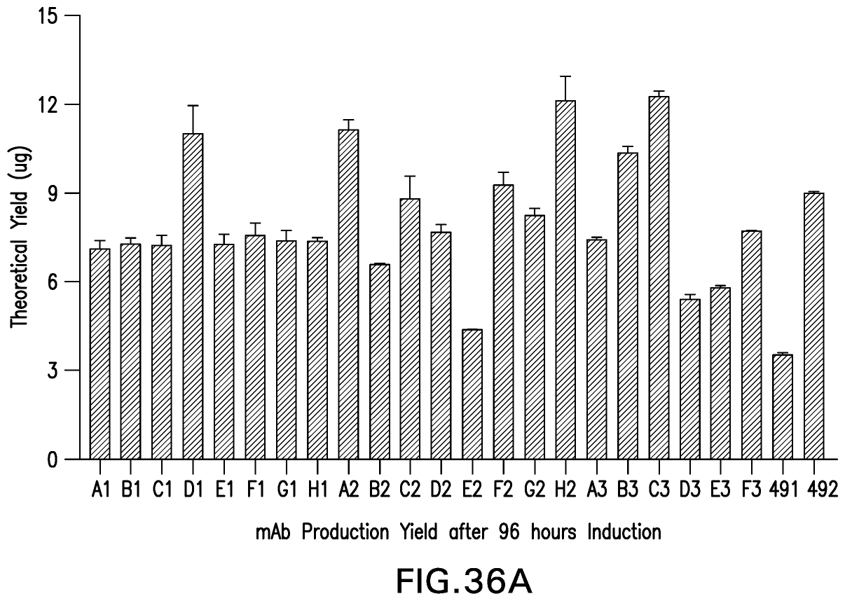

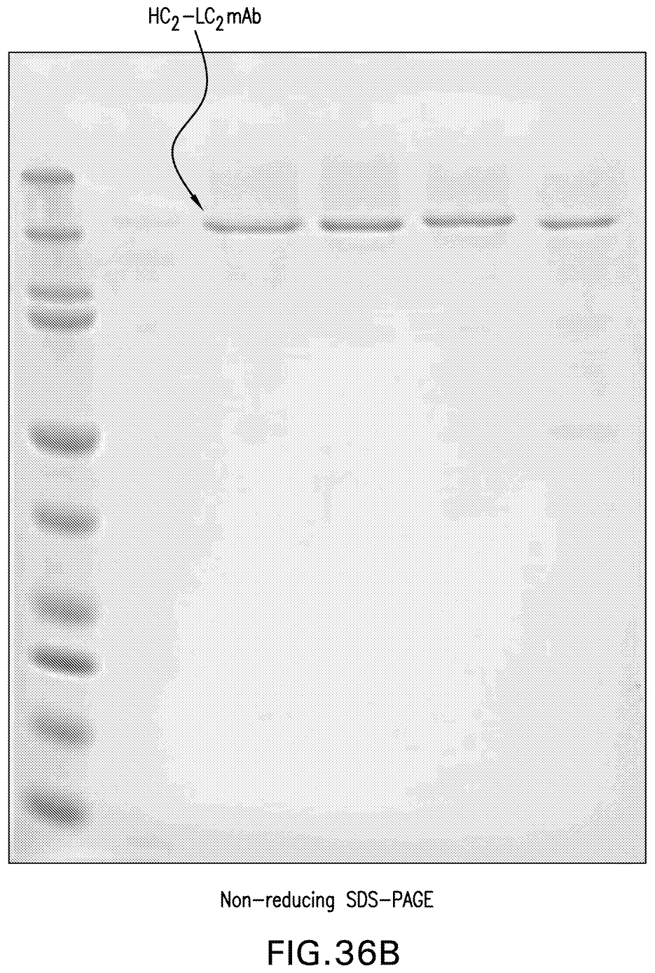

Following induction, culture supernatants were purified using Protein A and the antibody titer was quantified using Labchip CE-SDS assay, according to the manufacturer's protocol. FIG. 36A shows calculated antibody yield from the first 26 clones. Around 8 to 12 μg of purified antibody was obtained from each clone. FIG. 36B shows a non-reducing SDS-PAGE analysis of four randomly selected clones. The SDS-PAGE gel image reveals corrected folded, full antibody molecules (two heavy chain immunoglobulins and two light chain immunoglobulins (H2+L2), antibody tetramer: bivalent). This indicated that Fc-SED1.481 bait genetic construct did not interfere with the yeast ability to secrete fully-formed antibody tetramers (H2+L2).

TABLE of
Sequences

| SEQ | | |
|---|---|---|
| ID | | |
| NO: | Description | Sequence |
| 1 | DNA encoding Fc- | atgagatttccttcaattttttactgctgttttattcgcagcatcctccgcattagctgacaagacacata |
| | SED1.320 bait | cttgtccaccatgtccagctccagaattgttgggtggtccatccgttttcttgttcccaccaaagcca |
| | expression | aaggacactttgatgatctccagaactccagaggttacatgtgttgttgttgacgtttctcacgagg |
| | cassette | acccagaggttaagttcaactggtacgttgacggtgttgaagttcacaacgctaagactaagcca |
| | (ScSED1.320 | agagaagagcagtacgcttccacttacagagttgtttccgttttgactgttttgcaccaggactggtt |
| | Anchor) | gaacggtaaagaatacaagtgtaaggtttccaacaaggctttgccagctccaatcgaaaagacta |
| | | tctccaaggctaagggtcaaccaagagagccacaggtttacactttgccaccatccagagaaga |
| | | gatgactaagaaccaggtttccttgacttgtttggttaaaggattctacccatccgacattgctgttg |
| | | agtgggaatctaacggtcaaccagagaacaactacaagactactccaccagttttggattctgatg |
| | | gttccttcttcttgtactccaagttgactgttgacaagtccagatggcaacagggtaacgttttctcct |
| | | gttccgttatgcatgaggctttgcacaaccactacactcaaaagtccttgtctttgtcccctggtggt |
| | | ggtggtgtcgaccaattttctaattctacatcagcatcttcaacagacgtaacttccagttcttcaata |
| | | tcaacttccagtggttccgtcactatcacatcttcagaagctccagaaagtgataacggtacttcta |
| | | ctgcagcccctacagaaacctcaactgaagccccaaccactgctattcctactaatggtacatcta |
| | | ccgaagcaccaacaaccgccatacctacaaacggtacttctacagaagcaccaactgatactac |
| | | aaccgaagctccaactacagcattgcctacaaatggtacttctactgaagccccaactgacacca |
| | | ctacagaagctccaaccactggtttgcctacaaacggtacaacctcagcttttccacctactacatc |
| | | cttaccacctagtaataccactacaaccccaccttataacccatctactgattatactacagactaca |
| | | cagttgtaactgaatataccacttactgtccagaacctacaaccttcactacaaatggtaaaacata |
| | | caccgttactgaaccaaccactttaacaataaccgattgtccatgcacaatcgaaaagcctacaac |
| | | cacttctacaaccgaatacacagtcgttactgaatacactacatactgtccagaacctaccactttc |
| | | acaaccaatggtaaaacttacacagttaccgaaccaactacattgactattacagactgtccttgca |
| | | ctatagaaaagtcagaagctccagaatccagtgtacctgtcacagaatccaaaggtactactaca |
| | | aaggaaactggtgttaccactaaacaaacaaccgcaaatccatctttaacagtctcaactgtagtc |
| | | cctgtttcttcatccgccagttctcattcagttgtaattaattccaacggtgctaatgttgtcgttccag |
| | | gtgctttgggtttggcaggtgttgctatgttgtttttg |
| 2 | TetO7 promoter | ctcgatcgagtttaccactccctatcagtgatagagaaaagtgaaagtcgagtttaccactccctat |
| | DNA sequence | cagtgatagagaaaagtgaaagtcgagtttaccactccctatcagtgatagagaaaagtgaaagt |
| | | cgagtttaccactcctcagtgactatagagaaaagtgaaagtcgagtttaccactccctatcagtg |
| | | atagagaaaagtgaaagtcgagtttaccactccctatcagtgatagagaaaagtgaaagtcgagt |
| | | ttaccactccctatcagtgatagagaaaagtgaaagtcgagctcggtaccctatg |
| 3 | CYC-TT DNA | catgtaattagttatgtcacgcttacattcacgccctccccccacatccgctctaaccgaaaaggaa |
| | sequence | ggagttagacaacctgaagtctaggtccctatttatttttttatagttatgttagtattaagaacgttattt |
| | | atatttcaaatttttcttttttttctgtacagacgcgtgtacgcatgtaacattatactgaaaaccttgctt |
| | | gagaaggttttgggacgctcgaag |
| 4 | ScMET15 gene | atcctcatgaaaactgtgtaacataataaccgaagtgtcgaaaaggtggcaccttgtccaattgaa |
| | | cacgctcgatgaaaaaaataagatatatataaggttaagtaaagcgtctgttagaaaggaagttttt |
| | | cctttttcttgctctcttgtcttttcatctactatttccttcgtgtaatacagggtcgtcagatacatagat |
| | | acaattctattacccccatccatacaatgccatctcatttcgatactgttcaactacacgccggccaa |
| | | gagaaccctggtgacaatgctcacagatccagagctgtaccaatttacgccaccacttcttatgttt |
| | | tcgaaaactctaagcatggttcgcaattgtttggtctagaagttccaggttacgtctattcccgtttcc |
| | | aaaacccaaccagtaatgttttggaagaaagaattgctgctttagaaggtggtgctgctgctttggc |
| | | tgtttcctccggtcaagccgctcaaacccttgccatccaaggtttggcacacactggtgacaacat |
| | | cgtttccacttcttacttatacggtggtacttataaccagttcaaaatctcgttcaaaagatttggtatc |
| | | gaggctagatttgttgaaggtgacaatccagaagaattcgaaaaggtctttgatgaaagaaccaa |
| | | ggctgtttatttggaaaccattggtaatccaaagtacaatgttccggattttgaaaaaattgttgcaat |
| | | tgctcacaaacacggtattccagttgtcgttgacaacacatttggtgccggtggttacttctgtcagc |
| | | caattaaatacggtgctgatattgtaacacattctgctaccaaatggattggtggtcatggtactact |
| | | atcggtggtattattgttgactctggtaagttcccatggaaggactacccagaaaagttccctcaatt |
| | | ctctcaacctgccgaaggatatcacggtactatctacaatgaagcctacggtaacttggcatacat |
| | | cgttcatgttagaactgaactattaagagatttgggtccattgatgaacccatttgcctctttcttgcta |
| | | ctacaaggtgttgaaacattatctttgagagctgaaagacacggtgaaaatgcattgaagttagcc |
| | | aaatggttagaacaatccccatacgtatcttgggtttcataccctggtttagcatctcattctcatcat |
| | | gaaaatgctaagaagtatctatctaacggtttcggtggtgtcttatctttcggtgtaaaagacttacc |
| | | aaatgccgacaaggaaactgacccattcaaactttctggtgctcaagttgttgacaatttaaagctt |
| | | gcctctaacttggccaatgttggtgatgccaagaccttagtcattgctccatacttcactacccaca |
| | | aacaattaaatgacaaagaaaagttggcatctggtgttaccaaggacttaattcgtgtctctgttggt |
| | | atcgaatttattgatgacattattgcagacttccagcaatcttttgaaactgttttcgctggccaaaaa |
| | | ccatgagtgtgcgtaatgagttgtaaaattatgtataaacctactttctctcaca |
| 5 | ScURA3 gene | caatacagacgatgataacaaaccgaagttatctgatgtagaaaaggattaaagatgctaagaga |
| | | tagtgatgatatttcataaataatgtaattctatatatgttaattaccttttttgcgaggcatatttatggtg |
| | | aaggataagttttgaccatcaaagaaggttaatgtggctgtggtttcagggtccataaagcttttca |
| | | attcatcttttttttttttgttcttttttttgattccggtttctttgaaatttttttgattcggtaatctccgagca |
| | | gaaggaagaacgaaggaaggagcacagacttagattggtatatatacgcatatgtggtgttgaa |
| | | gaaacatgaaattgcccagtattcttaacccaactgcacagaacaaaaacctgcaggaaacgaa |
| | | gataaatcatgtcgaaagctacatataaggaagtgctgctactcatcctagtcctgttgctgccaa |
| | | gctatttaatatcatgcacgaaaagcaaacaaacttgtgtgcttcattggatgttcgtaccaccaag |
| | | gaattactggagttagttgaagcattaggtcccaaaatttgtttactaaaaacacatgtggatatcttg |
| | | actgatttttccatggagggcacagttaagccgctaaaggcattatccgccaagtacaattttttact |
| | | cttcgaagacagaaaatttgctgacattggtaatacagtcaaattgcagtactctgcgggtgtatac |
| | | agaatagcagaatgggcagacattacgaatgcacacggtgtggtgggcccaggtattgttagcg |
| | | gtttgaagcaggcggcggaagaagtaacaaaggaacctagaggccttttgatgttagcagaatt |
| | | gtcatgcaagggctccctagctactggagaatatactaagggtactgttgacattgcgaagagcg |
| | | acaaagattttgttatcggctttattgctcaaagagacatgggtggaagagatgaaggttacgattg |
| | | gttgattatgacacccggtgtgggtttagatgacaagggagacgcattgggtcaacagtatagaa |
| | | ccgtggatgatgtggtctctacaggatctgacattattattgttggaagaggactatttgcaaaggg |
| | | aagggatgctaaggtagagggtgaacgttacagaaaagcaggctgggaagcatatttgagaag |
| | | atgcggccagcaaaactaaaaaactgtattataagtaaatgcatgtatactaaactcacaaattaga |
| | | gcttcaatttaattatatcagttattacccgggaatctcggtcgtaatgatttctataatgacgaaaaa |
| | | aaaaaaattggaaagaaaaagcttcatggcctttataaaaaggaactatccaatacctcgccaga |
| | | accaagtaacagtattttacggggcacaaatcaagaacaataagacaggactgtaaagatggac |
| | | gcattgaactccaa |
| 6 | DNA encoding | atgagattcccatccatcttcactgctgttttgttcgctgcttcttctgctttggctgaggttcagttggt |
| | Anti-HER2 IgG1- | tgaatctggaggaggattggttcaacctggtggttctttgagattgtcctgtgctgcttccggtttca |
| | G1m(f), N297A | acatcaaggacacttacatccactgggttagacaagctccaggaaagggattggagtgggttgct |
| | expression | agaatctacccaactaacggttacacaagatacgctgactccgttaagggaagattcactatctct |
| | cassette | gctgacacttccaagaacactgcttacttgcagatgaactccttgagagctgaggatactgctgttt |
| | | actactgttccagatggggtggtgatggtttctacgctatggactactggggtcaaggaactttggt |
| | | tactgtctcgagtgcttcaactaagggaccatctgtcttcccattggctccatcttcaaagtctacttc |
| | | aggtggtactgctgctttgggttgcttggtcaaagactacttcccagagccagtcacagtttcttgg |
| | | aactctggtgctttgacttctggtgtccacactttcccagcagtcttacaatcttctggtttgtattcatt |
| | | gtcttctgttgttacagttccatcttcttctttgggtactcaaacttatatttgtaatgttaaccataaacc |
| | | atctaatactaaagttgataagaaagttgaaccaaaatcttgtgataagactcacacttgtccacctt |
| | | gcccagctccagaattgttaggtggtccttctgtcttcttgttcccaccaaagccaaaagatacattg |
| | | atgatttctagaactccagaggttacatgtgtcgtcgtcgatgtctctcacgaggatcctgaggtca |
| | | agttcaactggtacgtcgacggtgttgaggtccacaacgctaagactaagccaagagaagaaca |
| | | atacgcttctacatatagagttgtctctgttttgactgttttgcatcaggattggttaaatggtaaagaa |
| | | tataagtgtaaagtttctaataaggctttaccagcaccaattgaaaaaactatttctaaggctaaggg |
| | | tcaaccaagggaaccacaggtctacacattgccaccatcaagagaagaaatgactaaaaatcaa |
| | | gtttctttaacttgcttggttaagggtttctatccatcagatattgctgtcgagtgggaatctaatggtc |
| | | agccagaaaataattataaaacaactccaccagttttggattctgatggttctttctttttatattctaaa |
| | | ttgacagtcgataagtctaggtggcagcaaggtaacgttttctcatgctctgtcatgcacgaggctt |
| | | tgcacaaccactacacacaaaaatctttgtcattatctccaggt |
| 7 | Gal1 promoter | tgaagtacggattagaagccgccgagcgggtgacagccctccgaaggaagactctcctccgtg |
| | DNA sequence | cgtcctcgtcttcaccggtcgcgttcctgaaacgcagatgtgcctcgcgccgcactgctccgaac |
| | | aataaagattctacaatactagcttttatggttatgaagaggaaaaattggcagtaacctggcccca |
| | | caaaccttcaaatgaacgaatcaaattaacaaccataggatgataatgcgattagttttttagcctta |
| | | tttctggggtaattaatcagcgaagcgatgatttttgatctattaacagatatataaatgcaaaaactg |
| | | cataaccactttaactaatactttcaacattttcggtttgtattacttcttattcaaatgtaataaaagtat |
| | | caacaaaaaattgttaatatacctctatactttaacgtcaaggagaaaaaac |
| 8 | ScLEU2 gene | atgtctgcccctatgtctgcccctaagaagatcgtcgttttgccaggtgaccacgttggtcaagaa |
| | | atcacagccgaagccattaaggttcttaaagctatttctgatgttcgttccaatgtcaagttcgatttc |
| | | gaaaatcatttaattggtggtgctgctatcgatgctacaggtgtcccacttccagatgaggcgctg |
| | | gaagcctccaagaaggttgatgccgttttgttaggtgctgtgggtggtcctaaatggggtaccggt |
| | | agtgttagacctgaacaaggtttactaaaaatccgtaaagaacttcaattgtacgccaacttaagac |
| | | catgtaactttgcatccgactctcttttagacttatctccaatcaagccacaatttgctaaaggtactg |
| | | acttcgttgttgtcagagaattagtgggaggtatttactttggtaagagaaaggaagacgatggtg |
| | | atggtgtcgcttgggatagtgaacaatacaccgttccagaagtgcaaagaatcacaagaatggc |
| | | cgctttcatggccctacaacatgagccaccattgcctatttggtccttggataaagctaatgttttgg |
| | | cctcttcaagattatggagaaaaactgtggaggaaaccatcaagaacgaatttcctacattgaagg |
| | | ttcaacatcaattgattgattctgccgccatgatcctagttaagaacccaacccacctaaatggtatt |
| | | ataatcaccagcaacatgtttggtgatatcatctccgatgaagcctccgttatcccaggttccttgg |
| | | gtttgttgccatctgcgtccttggcctctttgccagacaagaacaccgcatttggtttgtacgaacca |
| | | tgccacggttctgctccagatttgccaaagaataaggtcaaccctatcgccactatcttgtctgctg |
| | | caatgatgttgaaattgtcattgaacttgcctgaagaaggtaaggccattgaagatgcagttaaaa |
| | | aggttttggatgcaggtatcagaactggtgatttaggtggttccaacagtaccaccgaagtcggtg |
| | | atgctgtcgccgaagaagttaagaaaatccttgc |
| 9 | DNA encoding | atgagattcccatccatcttcaccgccgttttgttcgctgcttcttccgctttggctgatattcaaatga |
| | MF-Pre-anti- | ctcaatctccatcttctttgtctgcttctgtcggtgatcgtgtcactattacttgtcgtgcttctcaagat |
| | HER2-Lc Kappa | gtcaacactgctgtcgcttggtatcaacagaagcccggtaaggctccaaagttgttgatttattctg |
| | CL expression | cttcttttttgtactctggtgtcccatctaggttctctggttctaggtctggtactgattttactttgactat |
| | cassette | ttcttctttgcaaccagaagattttgctacttattattgtcaacaacattatactacaccaccaacttttg |
| | | gtcaaggtaccaaggttgagatcaagagaactgttgctgctccatccgttttcattttcccaccatcc |
| | | gacgaacagttgaagtctggtacagcttccgttgtttgtttgttgaacaacttctacccaagagagg |
| | | ctaaggttcagtggaaggttgacaacgctttgcaatccggtaactcccaagaatccgttactgagc |
| | | aagactctaaggactccacttactccttgtcctccactttgactttgtccaaggctgattacgagaag |
| | | cacaaggtttacgcttgtgaggttacacatcagggtttgtcctccccagttactaagtccttcaaca |
| | | gaggagagtgt |
| 10 | TRP1 gene | atgtctgttattaatttcacaggtagttctggtccattggtgaaagtttgcggcttgcagagcacaga |
| | | ggccgcagaatgtgctctagattccgatgctgacttgctgggtattatatgtgtgcccaatagaaa |
| | | gagaacaattgacccggttattgcaaggaaaatttcaagtcttgtaaaagcatataaaaatagttca |
| | | ggcactccgaaatacttggttggcgtgtttcgtaatcaacctaaggaggatgttttggctctggtca |
| | | atgattacggcattgatatcgtccaactgcatggagatgagtcgtggcaagaataccaagagttcc |
| | | tcggtttgccagttattaaaagactcgtatttccaaaagactgcaacatactactcagtgcagcttca |
| | | cagaaacctcattcgtttattcccttgtttgattcagaagcaggtgggacaggtgaacttttggattg |
| | | gaactcgatttctgactgggttggaaggcaagagagccccgaaagcttacattttatgttagctgg |
| | | tggactgacgccagaaaatgttggtgatgcgcttagattaaatggcgttattggtgttgatgtaagc |
| | | ggaggtgtggagacaaatggtgtaaaagactctaacaaaatagcaaatttcgtcaaaaatgctaa |
| | | gaaa |
| 11 | DNA encoding | atgactttgtctttcgcacatttcacttatttatttactattttgttgggattgactaatattgctttggcttct |
| | AGA1 | gatccagaaactattttggttactattactaaaactaacgatgctaacggagtcgtcacaactactgt |
| | | ctcaccagctttggtctctacttctactattgtccaagctggtacaactactttgtatactacttggtgt |
| | | ccattaactgtttctacttcatcagcagcagaaatttctccatctatttcatacgctacaacattgtcaa |
| | | gattttcaactttaacattgtcaactgaggtttgctcacatgaggcttgcccttcttcatctactttacct |
| | | actacaacattgtcagttacttcaaagttcacttcttacatttgcccaacatgtcatactacagctattt |
| | | cttcattgtctgaagtcggtactacaactgttgtttcatcttctgctatagaaccatcttcagcttctatt |
| | | atatctccagttacatctactttgtcttcaacaacttcttctaaccctacaactacttctttgtcatcaac |
| | | atctacttctccatcttcaacttctacttctccatcatctacatctacttcatcttcttctacttctacttcat |
| | | cttcttctacttcaacttcatcttcttctacttctacttctccatcatctacatcaacatcatcatctttgac |
| | | atctacatcttcttcttcaacatctacttcacagtcttctacttctacatcttcttcatcaacttcaacatct |
| | | ccttcttcaacatctacatcttcttcttcaacatcaacatcaccatcatctaaatcaacttcagcttcatc |
| | | tacttctacatcttcttattctacttctacttctccatcattgacttcttcttcaccaacattggcttctacat |
| | | caccatcttctacttctatatcttctacatttactgattcaacttcttctttgggttcatcaatagcttcttct |
| | | tcaacatctgtttctttgtactctccatctactcccgtttactcagtcccttctacatcatctaacgttgct |
| | | actccttctatgacatcttctactgtcgagactactgtttcatctcaatcatcttctgaatatattactaa |
| | | atcttcaatttctactactattccatctttttctatgtcaacatatttcactacagtttctggtgttacaact |
| | | atgtacactacttggtgcccatactcttcagaatctgaaacttcaacattgacttctatgcatgaaac |
| | | agtcacaactgacgctactgtttgcacacacgagtcttgcatgccatctcagactacttcattaatta |
| | | cttcttctattaaaatgtctactaaaaatgttgctacttctgtttcaacatctacagttgaatcttcttacg |
| | | cttgttctacttgcgctgaaacttcacattcatattcttcagttcaaactgcttcttcttcttctgttactca |
| | | gcaaacaacttcaacaaaatcatgggtctcatctatgacaacatctgatgaagactttaacaaaca |
| | | cgctactggtaaatatcatgttacttcttctggaacttctacaatttctacatctgtttctgaagctacat |
| | | ctacttcttctatagattctgagtctcaagaacaatcttctcacttattatcaacatctgtcttgtcttcttc |
| | | ttctttatctgctacattgtcatctgactcaactattttgttattctcttcagtctcttctttgtcagttgaac |
| | | aatctccagtcactactttgcaaatatcatcaacttctgagattttacaaccaacatcttctacagctat |
| | | agctactatttctgcttctacttcttctttatctgctacttctatatctacaccatctacatctgttgaatct |
| | | actattgagtcttcttctttgacacctactgtttcttctattttcttatcttcttcttctgctccatcttctttgc |
| | | aaacatctgttacaactactgaagtttctactacatctatttcaattcaatatcagacttcttctatggtc |
| | | acaatttctcaatacatgggttctggttctcaaactagattgccattgggtaaattggtttttgctattat |
| | | ggcagtcgcttgtaatgttattttctct |
| 12 | DNA encoding Fc- | atgagatttccttcaatttttactgctgttttattcgcagcatcctccgcattagctgacaagacacata |
| | AGA2 bait | cttgtccaccatgtccagctccagaattgttgggtggtccatccgttttcttgttcccaccaaagcca |
| | expression | aaggacactttgatgatctccagaactccagaggttacatgtgttgttgttgacgtttctcacgagg |
| | cassette | acccagaggttaagttcaactggtacgttgacggtgttgaagttcacaacgctaagactaagcca |
| | | agagaagagcagtacgcttccacttacagagttgtttccgttttgactgttttgcaccaggactggtt |
| | | gaacggtaaagaatacaagtgtaaggtttccaacaaggctttgccagctccaatcgaaaagacta |
| | | tctccaaggctaagggtcaaccaagagagccacaggtttacactttgccaccatccagagaaga |
| | | gatgactaagaaccaggtttccttgacttgtttggttaaaggattctacccatccgacattgctgttg |
| | | agtgggaatctaacggtcaaccagagaacaactacaagactactccaccagttttggattctgatg |
| | | gttccttcttcttgtactccaagttgactgttgacaagtccagatggcaacagggtaacgttttctcct |
| | | gttccgttatgcatgaggctttgcacaaccactacactcaaaagtccttgtctttgtcccctaaagct |
| | | agcggtggtggtggttctgaagctgcagcaaaagaggcagctgctaagcaagagttgactacta |
| | | tttgtgaacaaattccatctccaactttggaatctactccatattctttgtctactactactattttggcta |
| | | atggtaaagctatgcaaggtgtttttgaatattataaatctgttactttcgtctctaactgcggttctcac |
| | | ccatctactacttctaagggttctccaattaatactcaatatgttttt |
| 13 | ScSED1.320 (1-18 | MKLSTVLLSAGLASTTLAQFSNSTSASSTDVTSSSSISTSSGSV |
| | SED1 signal | TITSSEAPESDNGTSTAAPTETSTEAPTTAIPTNGTSTEAPTTAI |
| | sequence) | PTNGTSTEAPTDTTTEAPTTALPTNGTSTEAPTDTTTEAPTTG |
| | | LPTNGTTSAFPPTTSLPPSNTTTTPPYNPSTDYTTDYTVVTEY |
| | | TTYCPEPTTFTTNGKTYTVTEPTTLTITDCPCTIEKPTTTSTTE |
| | | YTVVTEYTTYCPEPTTFTINGKTYTVTEPTTLTITDCPCTIEKS |
| | | EAPESSVPVTESKGTTTKETGVTTKQTTANPSLTVSTVVPVSS |
| | | SASSHSVVINSNGANVVVPGALGLAGVAMLFL |
| 14 | ScSED1.401 (1-18 | MKLSTVLLSAGLASTTLAQFSNSTSASSTDVTSSSSISTSSGSV |
| | SED1 signal | TITSSEAPESDNGTSTAAPTETSTEAPTTAIPTNGTSTEAPTDT |
| | sequence) | TTEAPTTALPTNGTSTEAPTDTTTEAPTTAIPTNGTSTEAPTDT |
| | | TTEAPTTALPTNGTSTEAPTDTTTEAPTTGLPTNGTTSAFPPTT |
| | | SLPPSNTTTTPPYNPSTDYTTDYTVVTEYTTYCPEPTTFTTNG |
| | | KTYTVTEPTTLTITDCPCTIEKPTTTSTTEYTVVTEYTTYCPEP |
| | | TTFTTNGKTYTVTEPTTLTITDCPCTIEKPTTTSTTEYTVVTEY |
| | | TTYCPEPTTFTTNGKTYTVTEPTTLTITDCPCTIEKSEAPESSV |
| | | PVTESKGTTTKETGVTTKQTTANPSLTVSTVVPVSSSASSHSV |
| | | VINSNGANVVVPGALGLAGVAMLFL |
| 15 | ScSED1.430 (1-18 | MKLSTVLLSAGLASTTLAQFSNSTSASSTDVTSSSSISTSSGSV |
| | SED1 signal | TITSSEAPESDNGTSTAAPTETSTEAPTTAIPTNGTSTEAPTDT |
| | sequence) | TSEAPTTALPTNGTSTEAPTDTTTEAPTTALPTNGTSTEAPTD |
| | | TTTEAPTTGLPTNGTTSAFPPTTSLPPSNTTTTPPYNPSTDYTT |
| | | DYTVVTEYTTYCPEPTTFTTNGKTYTVTEPTTLTITDCPCTIE |
| | | KPTTTSTTEYTVVTEYTTYCPEPTTFTTNGKTYTVTEPTTLTIT |
| | | DCPCTIEKPTTTSTTEYTVVTEYTTYCPEPTTFTINGKTYTVT |
| | | EPTTLTITDCPCTIEKPTTTSTTEYTVVTEYTTYCPEPTTFTTN |
| | | GKTYTVTEPTTLTITDCPCTIEKSEAPESSVPVTESKGTTTKET |
| | | GVTTKQTTANPSLTVSTVVPVSSSASSHSVVINSNGANVVVP |
| | | GALGLAGVAMLFL |
| 16 | ScSED1.481 (1-18 | MKLSTVLLSAGLASTTLAQFSNSTSASSTDATSSSSISTSSGSV |
| | SED1 signal | TITSSEAPESDNGTSTAAPTETSTEAPTTAIPTNGTSTEAPTDT |
| | sequence) | TTEAPTTALPTNGTSTEAPTDTTTEAPTTALPTNGTSTEAPTD |
| | | TTTEAPTTALPTNGTTSAFPPTTSLPPSNTTTTPPYNPSTDYTT |
| | | DYTVVTEYTTYCPEPTTFTTNGKTYTVTEPTTLTITDCPCTIE |
| | | KPTTTSTTEYTVVTEYTTYCPEPTTFTTNGKTYTVTEPTTLTIT |
| | | DCPCTIEKPTTTSTTEYTVVTEYTTYCPEPTTFTTNGKTYTVT |
| | | EPTTLTITDCPCTIEKPTTTSTTEYTVVTEYTTYCPEPTTFTTN |
| | | GKTYTVTEPTTLTITDCPCTIEKPTTTSTTEYTVVTEYTTYCPE |
| | | PTTFTTNGKTYTVTEPTTLTITDCPCTIEKSEAPESSVPVTESK |
| | | GTTTKETGVTTKQTTANPSLTVSTVVPVSSSASSHSVVINSNG |
| | | ANVVVPGALGLAGVAMLFL |
| 17 | ScSED1.320 | QFSNSTSASSTDVTSSSSISTSSGSVTITSSEAPESDNGTSTAAP |
| | (without SED1 | TETSTEAPTTAIPTNGTSTEAPTTAIPTNGTSTEAPTDTTTEAP |
| | signal sequence) | TTALPTNGTSTEAPTDTTTEAPTTGLPTNGTTSAFPPTTSLPPS |
| | | NTTTTPPYNPSTDYTTDYTVVTEYTTYCPEPTTFTTNGKTYT |
| | | VTEPTTLTITDCPCTIEKPTTTSTTEYTVVTEYTTYCPEPTTFT |
| | | TNGKTYTVTEPTTLTITDCPCTIEKSEAPESSVPVTESKGTTTK |
| | | ETGVTTKQTTANPSLTVSTVVPVSSSASSHSVVINSNGANVV |
| | | VPGALGLAGVAMLFL |
| 18 | ScSED1.401 | QFSNSTSASSTDVTSSSSISTSSGSVTITSSEAPESDNGTSTAAP |
| | (without SED1 | TETSTEAPTTAIPTNGTSTEAPTDTTTEAPTTALPTNGTSTEAP |
| | signal sequence) | TDTTTEAPTTAIPTNGTSTEAPTDTTTEAPTTALPINGTSTEAP |
| | | TDTTTEAPTTGLPTNGTTSAFPPTTSLPPSNTTTTPPYNPSTDY |
| | | TTDYTVVTEYTTYCPEPTTFTTINGKTYTVTEPTTLTITDCPCTI |
| | | EKPTTTSTTEYTVVTEYTTYCPEPTTFTTNGKTYTVTEPTTLTI |
| | | TDCPCTIEKPTTTSTTEYTVVTEYTTYCPEPTTFTTNGKTYTV |
| | | TEPTTLTITDCPCTIEKSEAPESSVPVTESKGTTTKETGVTTKQ |
| | | TTANPSLTVSTVVPVSSSASSHSVVINSNGANVVVPGALGLA |
| | | GVAMLFL |
| 19 | ScSED1.430 | QFSNSTSASSTDVTSSSSISTSSGSVTITSSEAPESDNGTSTAAP |
| | (without SED1 | TETSTEAPTTAIPTNGTSTEAPTDTTSEAPTTALPTNGTSTEAP |
| | signal sequence) | TDTTTEAPTTALPTNGTSTEAPTDTTTEAPTTGLPTNGTTSAF |
| | | PPTTSLPPSNTTTTPPYNPSTDYTTDYTVVTEYTTYCPEPTTFT |
| | | TNGKTYTVTEPTTLTITDCPCTIEKPTTTSTTEYTVVTEYTTY |
| | | CPEPTTFTINGKTYTVTEPTTLTITDCPCTIEKPTTTSTTEYTV |
| | | VTEYTTYCPEPTTFTTNGKTYTVTEPTTLTITDCPCTIEKPTTT |
| | | STTEYTVVTEYTTYCPEPTTFTTNGKTYTVTEPTTLTITDCPC |
| | | TIEKSEAPESSVPVTESKGTTTKETGVTTKQTTANPSLTVSTV |
| | | VPVSSSASSHSVVINSNGANVVVPGALGLAGVAMLFL |
| 20 | ScSED1.481 | QFSNSTSASSTDATSSSSISTSSGSVTITSSEAPESDNGTSTAAP |
| | (without SED1 | TETSTEAPTTAIPTNGTSTEAPTDTTTEAPTTALPTNGTSTEAP |
| | signal sequence) | TDTTTEAPTTALPTNGTSTEAPTDTTTEAPTTALPINGTTSAF |
| | | PPTTSLPPSNTTTTPPYNPSTDYTTDYTVVTEYTTYCPEPTTFT |
| | | TNGKTYTVTEPTTLTITDCPCTIEKPTTTSTTEYTVVTEYTTY |
| | | CPEPTTFTTNGKTYTVTEPTTLTITDCPCTIEKPTTTSTTEYTV |
| | | VTEYTTYCPEPTTFTTNGKTYTVTEPTTLTITDCPCTIEKPTTT |
| | | STTEYTVVTEYTTYCPEPTTFTTNGKTYTVTEPTTLTITDCPC |
| | | TIEKPTTTSTTEYTVVTEYTTYCPEPTTFTTNGKTYTVTEPTTL |
| | | TITDCPCTIEKSEAPESSVPVTESKGTTTKETGVTTKQTTANPS |
| | | LTVSTVVPVSSSASSHSVVINSNGANVVVPGALGLAGVAML |
| | | FL |
| 21 | DNA encoding | atgagatttccttcaatttttactgctgttttattcgcagcatcctccgcattagctgacaagacacata |
| | buman IgGI Fc | cttgtccaccatgtccagctccagaattgttgggtggtccatccgttttttgttcccaccaaagcca |
| | N297A mutein | aaggacactttgatgatctccagaactccagaggttacatgtgttgttgttgacgtttctcacgagg |
| | with S. | acccagaggttaagttcaactggtacgttgacggtgttgaagttcacaacgctaagactaagcca |
| | cerevisiae | agagaagagcagtacgcttccacttacagagttgtttccgttttgactgttttgcaccaggactggtt |
| | pre-pro alpha- | gaacggtaaagaatacaagtgtaaggtttccaacaaggctttgccagctccaatcgaaaagacta |
| | mating factor | tctccaaggctaagggtcaaccaagagagccacaggtttacactttgccaccatccagagaaga |
| | signal sequence | gatgactaagaaccaggtttccttgacttgtttggttaaaggattctacccatccgacattgctgttg |
| | at the 5′ end | agtgggaatctaacggtcaaccagagaacaactacaagactactccaccagttttggattctgatg |
| | | gttccttcttcttgtactccaagttgactgttgacaagtccagatggcaacagggtaacgttttctcct |
| | | gttccgttatgcatgaggctttgcacaaccactacactcaaaagtccttgtctttgtcccctggt |
| 22 | human IgG1 Fc | MRFPSIFTAVLFAASSALADKTHTCPPCPAPELLGGPSVFLFPP |
| | N297A mutein (C- | KPKDTLMISRTPEVTCVVVDVSHEDPEVKFNWYVDGVEVH |
| | terminal K-) | NAKTKPREEQYASTYRVVSVLTVLHQDWLNGKEYKCKVSN |
| | with 1-19 S. | KALPAPIEKTISKAKGQPREPQVYTLPPSREEMTKNQVSLTCL |
| | cerevisiae | VKGFYPSDIAVEWESNGQPENNYKTTPPVLDSDGSFFLYSKL |
| | pre-pro alpha- | TVDKSRWQQGNVFSCSVMHEALHNHYTQKSLSLSPG |
| | mating factor | |
| | signal sequence | |
| | at the 5′ end | |
| 23 | Linker | GGGVD |
| 24 | human IgG1 Fc | MRFPSIFTAVLFAASSALADKTHTCPPCPAPELLGGPSVFLFPP |
| | N297A mutein (C- | KPKDTLMISRTPEVTCVVVDVSHEDPEVKFNWYVDGVEVH |
| | terminal K-) | NAKTKPREEQYASTYRVVSVLTVLHQDWLNGKEYKCKVSN |
| | with GGGVD linker | KALPAPIEKTISKAKGQPREPQVYTLPPSREEMTKNQVSLTCL |
| | and 1-19 S. | VKGFYPSDIAVEWESNGQPENNYKTTPPVLDSDGSFFLYSKL |
| | cerevisiae | TVDKSRWQQGNVFSCSVMHEALHNHYTQKSLSLSPG |
| | pre-pro | GGGVD |
| | alpha-mating | |
| | factor signal | |
| | sequence | |
| 25 | S. cerevisiae | MRFPSIFTAVLFAASSALA |
| | pre-pro | |
| | alpha-mating | |
| | factor signal | |
| | sequence | |
| 26 | human IgG1 Fc | DKTHTCPPCPAPELLGGPSVFLFPPKPKDTLMISRTPEVTCVV |
| | N297A mutein (C- | VDVSHEDPEVKFNWYVDGVEVHNAKTKPREEQYASTYRVV |
| | terminal K-) | SVLTVLHQDWLNGKEYKCKVSNKALPAPIEKTISKAKGQPR |
| | without signal | EPQVYTLPPSREEMTKNQVSLTCLVKGFYPSDIAVEWESNGQ |
| | sequence | PENNYKTTPPVLDSDGSFFLYSKLTVDKSRWQQGNVFSCSV |
| | | MHEALHNHYTQKSLSLSPG |
| 27 | ScSED1.320 IgG1 | DKTHTCPPCPAPELLGGPSVFLFPPKPKDTLMISRTPEVTCVV |
| | Fc N297A mutein | VDVSHEDPEVKFNWYVDGVEVHNAKTKPREEQYASTYRVV |
| | (without signal | SVLTVLHQDWLNGKEYKCKVSNKALPAPIEKTISKAKGQPR |
| | sequence) | EPQVYTLPPSREEMTKNQVSLTCLVKGFYPSDIAVEWESNGQ |
| | (ScSED1.320 | PENNYKTTPPVLDSDGSFFLYSKLTVDKSRWQQGNVFSCSV |
| | Anchor) | MHEALHNHYTOKSLSLSPGGGGVDQFSNSTSASSTDVTSSSS |
| | | ISTSSGSVTITSSEAPESDNGTSTAAPTETSTEAPTTAIPTNGTS |
| | | TEAPTTAIPTNGTSTEAPTDTTTEAPTTALPTNGTSTEAPTDTT |
| | | TEAPTTGLPTNGTTSAFPPTTSLPPSNTTTTPPYNPSTDYTTDY |
| | | TVVTEYTTYCPEPTTFTINGKTYTVTEPTTLTITDCPCTIEKPT |
| | | TTSTTEYTVVTEYTTYCPEPTTFTTNGKTYTVTEPTTLTITDC |
| | | PCTIEKSEAPESSVPVTESKGTTTKETGVTTKQTTANPSLTVS |
| | | TVVPVSSSASSHSVVINSNGANVVVPGALGLAGVAMLFL |
| 28 | ScSED1.401 IgG1 | DKTHTCPPCPAPELLGGPSVFLFPPKPKDTLMISRTPEVTCVV |
| | Fc N297A mutein | VDVSHEDPEVKFNWYVDGVEVHNAKTKPREEQYASTYRVV |
| | (without signal | SVLTVLHQDWLNGKEYKCKVSNKALPAPIEKTISKAKGQPR |
| | sequence) | EPQVYTLPPSREEMTKNQVSLTCLVKGFYPSDIAVEWESNGQ |
| | (ScSED1.401 | PENNYKTTPPVLDSDGSFFLYSKLTVDKSRWQQGNVFSCSV |
| | Anchor) | MHEALHNHYTQKSLSLSPGGGGVDQFSNSTSASSTDVTSSSS |
| | | ISTSSGSVTITSSEAPESDNGTSTAAPTETSTEAPTTAIPTNGTS |
| | | TEAPTDTTTEAPTTALPTNGTSTEAPTDTTTEAPTTAIPTNGTS |
| | | TEAPTDTTTEAPTTALPTNGTSTEAPTDTTTEAPTTGLPTNGT |
| | | TSAFPPTTSLPPSNTTTTPPYNPSTDYTTDYTVVTEYTTYCPEP |
| | | TTFTTNGKTYTVTEPTTLTITDCPCTIEKPTTTSTTEYTVVTEY |
| | | TTYCPEPTTFTTNGKTYTVTEPTTLTITDCPCTIEKPTTTSTTE |
| | | YTVVTEYTTYCPEPTTFTTNGKTYTVTEPTTLTITDCPCTIEKS |
| | | EAPESSVPVTESKGTTTKETGVTTKQTTANPSLTVSTVVPVSS |
| | | SASSHSVVINSNGANVVVPGALGLAGVAMLFL |
| 29 | ScSED1.430 IgG1 | DKTHTCPPCPAPELLGGPSVFLFPPKPKDTLMISRTPEVTCVV |
| | Fc N297A mutein | VDVSHEDPEVKFNWYVDGVEVHNAKTKPREEQYASTYRVV |
| | (without signal | SVLTVLHQDWLNGKEYKCKVSNKALPAPIEKTISKAKGQPR |
| | sequence) | EPQVYTLPPSREEMTKNQVSLTCLVKGFYPSDIAVEWESNGQ |
| | (ScSED1.430 | PENNYKTTPPVLDSDGSFFLYSKLTVDKSRWQQGNVFSCSV |
| | Anchor) | MHEALHNHYTQKSLSLSPGGGGVDQFSNSTSASSTDVTSSSS |
| | | ISTSSGSVTITSSEAPESDNGTSTAAPTETSTEAPTTAIPTNGTS |
| | | TEAPTDTTSEAPTTALPTNGTSTEAPTDTTTEAPTTALPTNGT |
| | | STEAPTDTTTEAPTTGLPTNGTTSAFPPTTSLPPSNTTTTPPYN |
| | | PSTDYTTDYTVVTEYTTYCPEPTTFTTNGKTYTVTEPTTLTIT |
| | | DCPCTIEKPTTTSTTEYTVVTEYTTYCPEPTTFTTNGKTYTVT |
| | | EPTTLTITDCPCTIEKPTTTSTTEYTVVTEYTTYCPEPTTFTTN |
| | | GKTYTVTEPTTLTITDCPCTIEKPTTTSTTEYTVVTEYTTYCPE |
| | | PTTFTTNGKTYTVTEPTTLTITDCPCTIEKSEAPESSVPVTESK |
| | | GTTTKETGVTTKQTTANPSLTVSTVVPVSSSASSHSVVINSNG |
| | | ANVVVPGALGLAGVAMLFL |
| 30 | ScSED1.481 IgG1 | DKTHTCPPCPAPELLGGPSVFLFPPKPKDTLMISRTPEVTCVV |
| | Fc N297A mutein | VDVSHEDPEVKFNWYVDGVEVHNAKTKPREEQYASTYRVV |
| | (without signal | SVLTVLHQDWLNGKEYKCKVSNKALPAPIEKTISKAKGQPR |
| | sequence) | EPQVYTLPPSREEMTKNQVSLTCLVKGFYPSDIAVEWESNGQ |
| | (ScSED1.481 | PENNYKTTPPVLDSDGSFFLYSKLTVDKSRWQQGNVFSCSV |
| | Anchor) | MHEALHNHYTQKSLSLSPGGGGVDQFSNSTSASSTDATSSSS |
| | | ISTSSGSVTITSSEAPESDNGTSTAAPTETSTEAPTTAIPTNGTS |
| | | TEAPTDTTTEAPTTALPTNGTSTEAPTDTTTEAPTTALPTNGT |
| | | STEAPTDTTTEAPTTALPTNGTTSAFPPTTSLPPSNTTTTPPYN |
| | | PSTDYTTDYTVVTEYTTYCPEPTTFTTNGKTYTVTEPTTLTIT |
| | | DCPCTIEKPTTTSTTEYTVVTEYTTYCPEPTTFTTNGKTYTVT |
| | | EPTTLTITDCPCTIEKPTTTSTTEYTVVTEYTTYCPEPTTFTTN |
| | | GKTYTVTEPTTLTITDCPCTIEKPTTTSTTEYTVVTEYTTYCPE |
| | | PTTFTTNGKTYTVTEPTTLTITDCPCTIEKPTTTSTTEYTVVTE |
| | | YTTYCPEPTTFTTNGKTYTVTEPTTLTITDCPCTIEKSEAPESS |
| | | VPVTESKGTTTKETGVTTKQTTANPSLTVSTVVPVSSSASSHS |
| | | VVINSNGANVVVPGALGLAGVAMLFL |
| 31 | DNA encoding | atgagatttccttcaatttttactgctgttttattcgcagcatcctccgcattagctgacaagacacata |
| | ScSED1.481 IgG1 | cttgtccaccatgtccagctccagaattgttgggtggtccatccgttttcttgttcccaccaaagcca |
| | Fc N297A mutein | aaggacactttgatgatctccagaactccagaggttacatgtgttgttgttgacgtttctcacgagg |
| | (with 1-19 S. | acccagaggttaagttcaactggtacgttgacggtgttgaagttcacaacgctaagactaagcca |
| | cerevisiae | agagaagagcagtacgcttccacttacagagttgtttccgttttgactgttttgcaccaggactggtt |
| | pre-pro | gaacggtaaagaatacaagtgtaaggtttccaacaaggctttgccagctccaatcgaaaagacta |
| | alpha-mating | tctccaaggctaagggtcaaccaagagagccacaggtttacactttgccaccatccagagaaga |
| | factor signal | gatgactaagaaccaggtttccttgacttgtttggttaaaggattctacccatccgacattgctgttg |
| | sequence) | agtgggaatctaacggtcaaccagagaacaactacaagactactccaccagttttggattctgatg |
| | (ScSED1.481 | gttccttcttcttgtactccaagttgactgttgacaagtccagatggcaacagggtaacgttttctcct |
| | Anchor) | gttccgttatgcatgaggctttgcacaaccactacactcaaaagtccttgtctttgtcccctaaagct |
| | | agcggtggtggtggttctgaagcagctgcaaaagaagctgctgctaagcaattttctaattctactt |
| | | ctgcatcatcaacagacgctacttcttcttcttctatttctacttcttctggttctgttactattacttcttct |
| | | gaggcaccagagtctgacaacggtacatctactgctgctccaacagagacatccaccgaagca |
| | | ccaactactgctatacccactaatggtacttctactgaagctccaactgatacaactactgaggctc |
| | | ctaccaccgccttgccaacaaacggtacttcaactgaggctccaacagacacaaccactgaagc |
| | | acctactacagctttgccaactaacggtacctctacagaggccccaactgacactacaactgagg |
| | | cacccactactgccttaccaactaatggaactacttctgctttcccaccaactacttctttgccaccat |
| | | ctaacactactacaacaccaccatataacccatccactgattacactactgactacactgttgtcact |
| | | gaatacacaacctactgtcccgaaccaactacttttactaccaatggtaaaacatacacagttacc |
| | | gaaccaacaacattaaccattactgactgtccatgcactattgagaagccaaccactacctccaca |
| | | accgaatacactgttgttactgaatacactacttactgccccgaacctacaacatttactacaaacg |
| | | gcaaaacttatacagtcacagagccaactactttaaccattacagactgtccttgtactatcgaaaa |
| | | gcctacaacaacttctacaaccgaatataccgtcgtcaccgaatatacaacttattgcccagaacc |
| | | caccactttcacaaccaatggtaagacttataccgttactgagcctaccactttgactattactgatt |
| | | gcccatgtacaattgaaaaacctactactacttctactactgagtatacagtcgtcaccgagtatact |
| | | acttattgtcccgaacctactactttcaccactaatggcaaaacctatactgttaccgagcccacaa |
| | | ctttaactataactgattgcccttgcactattgaaaaaccaacaactacttctaccactgagtatactg |
| | | tcgttactgagtacaccacatattgtcccgaacctacaacctttactactaacggcaagacatacac |
| | | tgttacagagcccactactttaactatcaccgattgtccatgcaccatcgaaaaatctgaagctcca |
| | | gagtcttctgtcccagttacagaatctaaaggtactactacaaaggaaactggtgttactactaaac |
| | | aaactactgctaatccttctttgacagtttctacagttgttccagtctcttcttctgcttcttctcattctgt |
| | | tgttattaattctaacggtgcaaatgtcgttgttcccggtgctttgggtttggctggagttgctatgttg |
| | | tttttg |
| 32 | DNA encoding | agattcccatccatcttcactgctgttttgttcgctgcttcttctgctttggctgaagttcaattggttga |
| | adalimumab VH | atctggtggtggtttggttcagcccggtaggtctttgaggttgtcttgcgctgcatctggtttcacttt |
| | and an IgG1 Fc | cgacgactacgctatgcattgggtcagacaagctcccggtaagggtttggaatgggtttctgctat |
| | comprising the | tacttggaactctggtcatattgattatgctgattctgttgaaggtagatttactatttctagagataatg |
| | N297A mutation | ctaaaaactctttgtacttgcagatgaactctttgagggctgaggacactgctgtctactactgtgct |
| | (with 1-19 S. | aaggtttcttatttgtctactgcttcttctttggactactggggtcaaggtactttggttactgtctcgag |
| | cerevisiae | tgcttcaactaagggaccatctgtcttcccattggctccatcttcaaagtctacttcaggtggtactg |
| | pre-pro | ctgctttgggttgcttggtcaaagactacttcccagagccagtcacagtttcttggaactctggtgct |
| | alpha-mating | ttgacttctggtgtccacactttcccagcagtcttacaatcttctggtttgtattcattgtcttctgttgtta |
| | factor signal | cagttccatcttcttctttgggtactcaaacttatatttgtaatgttaaccataaaccatctaatactaaa |
| | sequence) | gttgataagaaagttgaaccaaaatcttgtgataagactcacacttgtccaccttgcccagctccag |
| | | aattgttaggtggtccttctgtcttcttgttcccaccaaagccaaaagatacattgatgatttctagaa |
| | | ctccagaggttacatgtgtcgtcgtcgatgtctctcacgaggatcctgaggtcaagttcaactggta |
| | | cgtcgacggtgttgaggtccacaacgctaagactaagccaagagaagaacaatacgcttctaca |
| | | tatagagttgtctctgttttgactgttttgcatcaggattggttaaatggtaaagaatataagtgtaaag |
| | | tttctaataaggctttaccagcaccaattgaaaaaactatttctaaggctaagggtcaaccaaggga |
| | | accacaggtctacacattgccaccatcaagagaagaaatgactaaaaatcaagtttctttaacttgc |
| | | ttggttaagggtttctatccatcagatattgctgtcgagtgggaatctaatggtcagccagaaaata |
| | | attataaaacaactccaccagttttggattctgatggttcttttttttatattctaaattgacagtcgata |
| | | agtctaggtggcagcaaggtaacgttttctcatgctctgtcatgcacgaggctttgcacaaccact |
| | | acacacaaaaatctttgtcattatctccaggt |
| 33 | DNA encoding | atgagattcccatccatcttcaccgccgttttgttcgctgcttcttccgctttggctgatattcaaatga |
| | adalimumab VL- | ctcaatctccatcttctttgtctgcttctgtcggtgatcgtgtcactattacttgtcgtgcttctcaaggt |
| | Kappa (with 1-19 | attagaaactacttggcttggtatcaacagaagcccggtaaggctccaaagttgttgatttacgctg |
| | S. cerevisiae | cttctactttgcagtctggtgtcccatctaggttctctggttctggttctggtactgattttactttgacta |
| | pre-pro | tttcttctttgcaaccagaagatgttgctacttattattgtcaaagatataatagagctccatatactttt |
| | alpha-mating | ggtcaaggtaccaaggttgagatcaagagaactgttgctgctccatccgttttcattttcccaccat |
| | factor signal | ccgacgaacagttgaagtctggtacagcttccgttgtttgtttgttgaacaacttctacccaagaga |
| | sequence) | ggctaaggttcagtggaaggttgacaacgctttgcaatccggtaactcccaagaatccgttactga |
| | | gcaagactctaaggactccacttactccttgtcctccactttgactttgtccaaggctgattacgaga |
| | | agcacaaggtttacgcttgtgaggttacacatcagggtttgtcctccccagttactaagtccttcaa |
| | | cagaggagagtgt |
| 34 | SED1-FLOS-680 | QFSNSTSASSTDVTSSSSISTSSGSVTITSSEAPESDNGTSTAAP |
| | without signal | TETSTEAPTTAIPTNGTSTEAPTTAIPTNGTSTEAPTDTTTEAP |
| | sequence | TTALPTNGTSTEAPTDTTTEAPTTGLPTNGTTTEPWTGTFTST |
| | | STEMTTITDTNGQLTDETVIVIRTPTTASTITTTTEPWTGTFTS |
| | | TSTEMTTVTGINGQPTDETVIVIRTPTSEGLITTTTEPWTGTFT |
| | | STSTEMTTVTGTNGQPTDETVIVIRTPTSEGLITTTTEPWTGTF |
| | | TSTSTEVTTITGINGQPTDETVIVIRTPTSEGLITTTTEPWTGTF |
| | | TSTSTEMTTVTGTNGQPTDETVIVIRTPTSEGLISTTTEPWTGT |
| | | FTSTSTEVTTITGTNGQPTDETVIVIRTPTSEGLITTTTEPWTGT |
| | | FTSTSTEMTTVTGTNGQPTDETVIVIRTPTSEGLITRTTEPWTG |
| | | TFTSTSTEVTTITGTNGQPTDETVIVIRTPTTAISSSTTSAFPPTT |
| | | SLPPSNTTTTPPYNPSTDYTTDYTVVTEYTTYCPEPTTFTING |
| | | KTYTVTEPTTLTITDCPCTIEKPTTTSTTEYTVVTEYTTYCPEP |
| | | TTFTTNGKTYTVTEPTTLTITDCPCTIEKSEAPESSVPVTESKG |
| | | TTTKETGVTTKQTTANPSLTVSTVVPVSSSASSHSVVINSNGA |
| | | NVVVPGALGLAGVAMLFL |
| | | QFSNSTSASSTDVTSSSSISTSSGSVTITSSEAPESDNGTSTAAP |
| | | TETSTEAPTTAIPTNGTSTEAPTTAIPTNGTSTEAPTDTTTEAP |
| 35 | SED1-FLO1-660 | TTALPTNGTSTEAPTDTTTEAPTTGLPTNGTTTEPWTGTFTST |
| | without signal | STEMTTITGTNGVPTDETVIVIRTPTSEGLISTTTEPWTGTFTS |
| | sequence | TSTEMTTITGTNGQPTDETVIVIRTPTSEGLISTTTEPWTGTFT |
| | | STSTEMTHVTGTNGVPTDETVIVIRTPTSEGLISTTTEPWTGTF |
| | | TSTSTEVTTITGTNGQPTDETVIVIRTPTSEGLISTTTEPWTGTF |
| | | TSTSTEMTTVTGINGQPTDETVIVIRTPTSEGLVTTTTEPWTG |
| | | TFTSTSTEMSTVTGTNGLPTDETVIVVKTPTTAISSSLSSSSSG |
| | | QITSSITSSRPIITPFYPSNGTSVISSSVISSSVTSSLFTSSPVISSS |
| | | VISSSTTTSTSIFSETTSAFPPTTSLPPSNTTTTPPYNPSTDYTTD |
| | | YTVVTEYTTYCPEPTTFTTNGKTYTVTEPTTLTITDCPCTIEKP |
| | | TTTSTTEYTVVTEYTTYCPEPTTFTTNGKTYTVTEPTTLTITD |
| | | CPCTIEKSEAPESSVPVTESKGTTTKETGVTTKQTTANPSLTV |
| | | STVVPVSSSASSHSVVINSNGANVVVPGALGLAGVAMLFL |
| 36 | DNA encoding | atgagatttccttcaatttttactgctgttttattcgcagcatcctccgcattagctgacaagacacata |
| | SED1-FLO5-680 | cttgtccaccatgtccagctccagaattgttgggtggtccatccgttttcttgttcccaccaaagcca |
| | fusion anchor | aaggacactttgatgatctccagaactccagaggttacatgtgttgttgttgacgtttctcacgagg |
| | (IgG1 (N297A) | acccagaggttaagttcaactggtacgttgacggtgttgaagttcacaacgctaagactaagcca |
| | with 1-19 S. | agagaagagcagtacgcttccacttacagagttgtttccgttttgactgttttgcaccaggactggtt |
| | cerevisiae | gaacggtaaagaatacaagtgtaaggtttccaacaaggctttgccagctccaatcgaaaagacta |
| | pre-pro | tctccaaggctaagggtcaaccaagagagccacaggtttacactttgccaccatccagagaaga |
| | alpha-mating | gatgactaagaaccaggtttccttgacttgtttggttaaaggattctacccatccgacattgctgttg |
| | factor signal | agtgggaatctaacggtcaaccagagaacaactacaagactactccaccagttttggattctgatg |
| | sequence and | gttccttcttcttgtactccaagttgactgttgacaagtccagatggcaacagggtaacgttttctcct |
| | linker) | gttccgttatgcatgaggctttgcacaaccactacactcaaaagtccttgtctttgtcccctaaagct |
| | | agcggaggtggaggttctgaagccgcggcgaaagaggcagctgccaaacagtttagcaatagt |
| | | acgagcgcctcctcaaccgatgtgacgtcatcaagctccattagtacaagtagtggcagcgtcac |
| | | aataactagtagcgaggcacccgagtcagacaatggcaccagtacagcagctcctaccgagac |
| | | tagtacagaggccccgaccacagctatcccaaccaatggcacatccaccgaagcccccactac |
| | | ggcgattcccacgaacggtacgtccacggaagctcccacagacaccactacagaggcgccta |
| | | cgaccgccctacctactaatgggaccagtacggaagccccaacggatactaccacagaggcac |
| | | ccacgacgggactaccaacaaacggcactaccacggagccgtggaccggtacctttacgagc |
| | | acgtctaccgaaatgactactatcacggacaccaatggtcaacttacggatgagacggtgattgt |
| | | gataagaactccaactaccgcaagcactatcaccacaaccacggaaccttggactggaactttca |
| | | catctacctccaccgagatgactaccgtaacgggcacgaatggacaaccgactgacgaaactgt |
| | | aatagtcatccgtacgcccacgtcagagggcttaattacaactacaaccgagccgtggacggga |
| | | acattcaccagtacaagcacagagatgaccacggtaaccggcacaaacgggcagcctacagat |
| | | gagacagtaatcgttatacgtacacccacctccgaaggattgataactactacaaccgaaccctg |
| | | gacgggaaccttcacctctacgagtactgaagtaacgaccataactgggacaaatggccaacct |
| | | actgatgaaacagtcatcgtaatcaggaccccgaccagcgagggtttgattacgaccaccacgg |
| | | agccatggacaggaacttttactagcacaagtaccgaaatgactactgtcactggaactaacggc |
| | | cagccgactgacgagacggtaattgtaattaggacccccactagtgagggtctaataagtacaac |
| | | tactgaaccttggacggggacgttcacatccacgagcacagaggtcaccacaattacagggac |
| | | caacggacagccaactgatgagactgtgatcgttattaggaccccaacatctgaagggttaatca |
| | | ccacgaccaccgagccgtggacaggtacatttacctctaccagcacagagatgacaactgtaac |
| | | aggtactaatggacaaccgactgacgagacagtcatcgtcatccgaacccccacgagtgaagg |
| | | cttgattacgcgaacaacggaaccctggaccggcaccttcacaagtacgtcaacagaggtcact |
| | | acgattacgggaactaatgggcagccaactgatgagacagtcatcgtaattcggacgccgacca |
| | | cggccatcagcagcagtacgacctctgcattcccccctactaccagcttgccaccgagcaatact |
| | | actactactcccccgtataaccctagtacagattataccacagattacactgtagttaccgagtaca |
| | | ccacatactgtcccgaaccaactacgtttaccaccaatgggaaaacgtacaccgtgaccgaacc |
| | | gacaactctcactattactgactgcccatgtactattgagaagcccaccacgacttcaaccactga |
| | | atataccgtagtgactgaatatacaacctactgccctgaacctacaacattcaccactaatggcaa |
| | | gacttacaccgtgacggaacctacaacattgacaattaccgactgtccttgcactatcgagaaatc |
| | | tgaggccccagaaagttctgtcccagttactgaatccaaggggacgacaactaaggagactgg |
| | | ggtaacgaccaagcagacaactgccaacccaagtctcaccgtaagcactgtggtaccggtaagt |
| | | agttcagcgagtagtcactctgttgtaatcaactccaatggcgccaacgttgtagtaccgggggc |
| | | acttggtcttgctggggttgcgatgctgttcttg |
| 37 | SED1-FLO5-680 | MRFPSIFTAVLFAASSALADKTHTCPPCPAPELLGGPSVFLFPP |
| | fusion anchor | KPKDTLMISRTPEVTCVVVDVSHEDPEVKFNWYVDGVEVH |
| | (IgG1 (N297A) | NAKTKPREEQYASTYRVVSVLTVLHQDWLNGKEYKCKVSN |
| | with 1-19 S. | KALPAPIEKTISKAKGQPREPQVYTLPPSREEMTKNQVSLTCL |
| | cerevisiae | VKGFYPSDIAVEWESNGQPENNYKTTPPVLDSDGSFFLYSKL |
| | pre-pro | TVDKSRWQQGNVFSCSVMHEALHNHYTQKSLSLSPKASGG |
| | alpha-mating | GGSEAAAKEAAAKOFSNSTSASSTDVTSSSSISTSSGSVTITSS |
| | factor signal | EAPESDNGTSTAAPTETSTEAPTTAIPTNGTSTEAPTTAIPTNG |
| | sequence and | TSTEAPTDTTTEAPTTALPTNGTSTEAPTDTTTEAPTTGLPTN |
| | linker) | GTTTEPWTGTFTSTSTEMTTITDINGQLTDETVIVIRTPTTAST |
| | | ITTTTEPWTGTFTSTSTEMTTVTGTNGQPTDETVIVIRTPTSEG |
| | | LITTTTEPWTGTFTSTSTEMTTVTGTNGQPTDETVIVIRTPTSE |
| | | GLITTTTEPWTGTFTSTSTEVTTITGTNGQPTDETVIVIRTPTSE |
| | | GLITTTTEPWTGTFTSTSTEMTTVTGTNGQPTDETVIVIRTPTS |
| | | EGLISTTTEPWTGTFTSTSTEVTTITGTNGQPTDETVIVIRTPTS |
| | | EGLITTTTEPWTGTFTSTSTEMTTVTGINGQPTDETVIVIRTPT |
| | | SEGLITRTTEPWTGTFTSTSTEVTTITGTNGQPTDETVIVIRTPT |
| | | TAISSSTTSAFPPTTSLPPSNTTTTPPYNPSTDYTTDYTVVTEY |
| | | TTYCPEPTTFTTNGKTYTVTEPTTLTITDCPCTIEKPTTTSTTE |
| | | YTVVTEYTTYCPEPTTFTTNGKTYTVTEPTTLTITDCPCTIEKS |
| | | EAPESSVPVTESKGTTTKETGVTTKQTTANPSLTVSTVVPVSS |
| | | SASSHSVVINSNGANVVVPGALGLAGVAMLFL |
| 38 | SED1-FLO5-680 | DKTHTCPPCPAPELLGGPSVFLFPPKPKDTLMISRTPEVTCVV |
| | fusion anchor | VDVSHEDPEVKFNWYVDGVEVHNAKTKPREEQYASTYRVV |
| | (IgG1 (N297A) | SVLTVLHQDWLNGKEYKCKVSNKALPAPIEKTISKAKGQPR |
| | | EPQVYTLPPSREEMTKNQVSLTCLVKGFYPSDIAVEWESNGQ |
| | | PENNYKTTPPVLDSDGSFFLYSKLTVDKSRWQQGNVFSCSV |
| | | MHEALHNHYTOKSLSLSPKASGGGGSEAAAKEAAAKQFSNS |
| | | TSASSTDVTSSSSISTSSGSVTITSSEAPESDNGTSTAAPTETST |
| | | EAPTTAIPTNGTSTEAPTTAIPTNGTSTEAPTDTTTEAPTTALP |
| | | TNGTSTEAPTDTTTEAPTTGLPTNGTTTEPWTGTFTSTSTEMT |
| | | TITDINGQLTDETVIVIRTPTTASTITTTTEPWTGTFTSTSTEM |
| | | TTVTGTNGQPTDETVIVIRTPTSEGLITTTTEPWTGTFTSTSTE |
| | | MTTVTGTNGQPTDETVIVIRTPTSEGLITTTTEPWTGTFTSTST |
| | | EVTTITGTNGQPTDETVIVIRTPTSEGLITTTTEPWTGTFTSTST |
| | | EMTTVTGTNGQPTDETVIVIRTPTSEGLISTTTEPWTGIFTSTS |
| | | TEVTTITGTNGQPTDETVIVIRTPTSEGLITTTTEPWTGTFTSTS |
| | | TEMTTVTGINGQPTDETVIVIRTPTSEGLITRTTEPWTGTFTST |
| | | STEVTTITGTNGQPTDETVIVIRTPTTAISSSTTSAFPPTTSLPPS |
| | | NTTTTPPYNPSTDYTTDYTVVTEYTTYCPEPTTFTINGKTYT |
| | | VTEPTTLTITDCPCTIEKPTTTSTTEYTVVTEYTTYCPEPTTFT |
| | | TNGKTYTVTEPTTLTITDCPCTIEKSEAPESSVPVTESKGTTTK |
| | | ETGVTTKQTTANPSLTVSTVVPVSSSASSHSVVINSNGANVV |
| | | VPGALGLAGVAMLFL |
| 39 | DNA encoding | atgagatttccttcaatttttactgctgitttattcgcagcatcctccgcattagetgacaagacacata |
| | SED1-FLO1-660 | cttgtccaccatgtccagctccagaattgttgggggtccatccgttttcttgttoccaccaaagcca |
| | fusion anchor | aaggacactitgatgatctccagaactccagaggttacatgtgttgttgttgacgtttctcacgagg |
| | (with 1-19 S. | acccagaggttaagttcaactggtacgttgacggtgttgaagttcacaacgctaagactaagcca |
| | cerevisiae | agagaagagcagtacgcttccacttacagagitgtttecgttttgactgttttgcaccaggactggtt |
| | pre-pro | gaacggtaaagaatacaagtgtaaggtttccaacaaggctttgccagctccaatcgaaaagacta |
| | alpha-mating | tctccaaggctaagggtcaaccaagagagccacaggittacactitgccaccatocagagaaga |
| | factor signal | gatgactaagaaccaggtttccttgacttgtttggttaaaggattctacccatccgacattgctgttg |
| | sequence) | agtgggaatctaacggtcaaccagagaacaactacaagactactccaccagttitggattetgatg |
| | | gttccttcttcttgtactccaagttgactgttgacaagtccagatggcaacagggtaacgttttetect |
| | | gttccgttatgcatgaggetttgcacaaccactacactcaaaagtecttgtettigtcccctaaaget |
| | | agcggaggaggtggaagcgaagctgccgctaaggaggcagccgccaagcagttttctaattcc |
| | | acgagegegtctagtaccgacgtgacatetagitcatecatcagtactagcageggetcegtcac |
| | | tataactagctctgaggcaccegagagegacaatggtacatcaacagetgcccctacagaaactt |
| | | ctacggaggcccccacgacegocatacctaccaatggcaccagtactgaggegcetactaccg |
| | | caatcccgacaaacggaaccagcacagaagctccaactgatacaacaacagaagccccaaca |
| | | acagcactgcegacgaaeggcacgtetaccgaagetcccacggacacgacaactgaggegcc |
| | | gactacaggattacccacaaatgggactaccaccgagcettggacagggaccttcacctctacg |
| | | tctaccgaaatgacaacaataaccggtacgaacggagtaccaactgacgagactgtcattgttatt |
| | | cgtacaccaacaagegaaggtcttataagcacgacgactgagccatggactgggacitttacga |
| | | gtacgagtacggagatgactactataacaggcaccaatggccagcccactgacgagaccgtca |
| | | tagttataaggactcctacctcagaaggattaatctctacaacaacagagccatggacgggcacc |
| | | ttcacttcaacgtcaactgagatgacacatgtgacaggtacgaatggtgtaccaaccgacgagac |
| | | ggtcatcgtgattaggactcccacaagegaggggctgatatcaacaactactgaaccctggacg |
| | | ggtacgttcactagtacatccacegaagttacgactataaceggaacgaacgggcaacccactg |
| | | atgaaacegttattgtgatcaggacgcctacttccgaaggtctgatatctaccacgactgagccct |
| | | ggacgggcacgttcacttcaacttocactgaaatgacgacggtgactggaaccaacggtcaacc |
| | | gaccgacgaaaccgtcatagtgattaggacacctaccagtgagggactagttaccactaccacg |
| | | gaaccatggactggtacatttacatcaacatecaccgagatgtctacagttacgggcacaaatgg |
| | | cttgcctacagatgaaaccgtgatagttgttaaaacgcccaccactgcaatttcaagcagettgtet |
| | | agctctagctceggacaaattaccagttccataacatctagccgtccaattataacaccattttatec |
| | | aagcaacggcactagtgtgatttcctccagtgttatatcatctagtgttacgtcatcactattcacatc |
| | | atcccctgtcatetettcateogttatctectotictacgaccacgtcaacgtcaatttttagtgaaaca |
| | | acctctgcatttcccccgacgacgtetctacctccaagtaatacgactactactcccccttataatcc |
| | | gagcacagactacacaactgactataccgtegttacggagtacacgacgtattgccctgaaccca |
| | | caacctttactactaatgggaaaacttacactgteactgaaccgaccacgctaacgattacagact |
| | | gcccctgcacgatagaaaaacctaccaccacttcaaccacggagtacactgtggtgactgaata |
| | | cacgacatactgtccagaacctacaaccttcacaaccaacggaaaaacatacacggtgacgga |
| | | acctacaacgctgacgatcactgattgcccatgtacaatcgagaaatctgaagctccagaatcatc |
| | | tgtcccggttactgaatcaaagggtactacaaccaaggaaactggggtcactacaaaacaaacc |
| | | acggcaaatccgagtttgacagtcagtacggtagtgcctgtgtctagtagcgccagttcacacag |
| | | cgtagtgataaattctaatggggcgaacgtggttgtacctggogcgttaggcttggceggggtag |
| | | ccatgctttttttg |
| 40 | SED1-FLO1-660 | MRFPSIFTAVLFAASSALADKTHTCPPCPAPELLGGPSVFLFPP |
| | fusion anchor | KPKDTLMISRTPEVTCVVVDVSHEDPEVKFNWYVDGVEVH |
| | (with 1-19 S. | NAKTKPREEQYASTYRVVSVLTVLHQDWLNGKEYKCKVSN |
| | cerevisiae | KALPAPIEKTISKAKGQPREPQVYTLPPSREEMTKNQVSLTCL |
| | pre-pro | VKGFYPSDIAVEWESNGQPENNYKTTPPVLDSDGSFFLYSKL |
| | alpha-mating | TVDKSRWQQGNVFSCSVMHEALHNHYTQKSLSLSPKASGG |
| | factor signal | GGSEAAAKEAAAKQFSNSTSASSTDVTSSSSISTSSGSVTITSS |
| | sequence) | EAPESDNGTSTAAPTETSTEAPTTAIPTNGTSTEAPTTAIPING |
| | | TSTEAPTDTTTEAPTTALPTNGTSTEAPTDTTTEAPTTGLPTN |
| | | GTTTEPWTGTFTSTSTEMTTITGTNGVPTDETVIVIRTPTSEGL |
| | | ISTTTEPWTGTFTSTSTEMTTITGTNGQPTDETVIVIRTPTSEG |
| | | LISTTTEPWTGTFTSTSTEMTHVTGINGVPTDETVIVIRTPTSE |
| | | GLISTTTEPWTGTFTSTSTEVTTITGTNGQPTDETVIVIRTPTSE |
| | | GLISTTTEPWTGTFTSTSTEMTTVTGINGQPTDETVIVIRTPTS |
| | | EGLVTTTTEPWTGTFTSTSTEMSTVTGTNGLPTDETVIVVKTP |
| | | TTAISSSLSSSSSGQITSSITSSRPIITPFYPSNGTSVISSSVISSSV |
| | | TSSLFTSSPVISSSVISSSTTTSTSIFSETTSAFPPTTSLPPSNTTT |
| | | TPPYNPSTDYTTDYTVVTEYTTYCPEPTTFTTNGKTYTVTEPT |
| | | TLTITDCPCTIEKPTTTSTTEYTVVTEYTTYCPEPTTFTTNGKT |
| | | YTVTEPTTLTITDCPCTIEKSEAPESSVPVTESKGTTTKETGVT |
| | | TKQTTANPSLTVSTVVPVSSSASSHSVVINSNGANVVVPGAL |
| | | GLAGVAMLFL |
| 41 | SED1-FLO1-660 | DKTHTCPPCPAPELLGGPSVFLFPPKPKDTLMISRTPEVTCVV |
| | fusion anchor | VDVSHEDPEVKFNWYVDGVEVHNAKTKPREEQYASTYRVV |
| | | SVLTVLHQDWLNGKEYKCKVSNKALPAPIEKTISKAKGQPR |
| | | EPQVYTLPPSREEMTKNQVSLTCLVKGFYPSDIAVEWESNGQ |
| | | PENNYKTTPPVLDSDGSFFLYSKLTVDKSRWQQGNVFSCSV |
| | | MHEALHNHYTOKSLSLSPKASGGGGSEAAAKEAAAKQFSNS |
| | | TSASSTDVTSSSSISTSSGSVTITSSEAPESDNGTSTAAPTETST |
| | | EAPTTAIPTNGTSTEAPTTAIPTNGTSTEAPTDTTTEAPTTALP |
| | | TNGTSTEAPTDTTTEAPTTGLPTNGTTTEPWTGTFTSTSTEMT |
| | | TITGTNGVPTDETVIVIRTPTSEGLISTTTEPWTGTFTSTSTEM |
| | | TTITGTNGQPTDETVIVIRTPTSEGLISTTTEPWTGTFTSTSTE |
| | | MTHVTGTNGVPTDETVIVIRTPTSEGLISTTTEPWTGTFTSTST |
| | | EVTTITGTNGQPTDETVIVIRTPTSEGLISTTTEPWTGTFTSTST |
| | | EMTTVTGTNGQPTDETVIVIRTPTSEGLVTTTTEPWTGTFTST |
| | | STEMSTVTGTNGLPTDETVIVVKTPTTAISSSLSSSSSGQITSSI |
| | | TSSRPIITPFYPSNGTSVISSSVISSSVTSSLFTSSPVISSSVISSST |
| | | TTSTSIFSETTSAFPPTTSLPPSNTTTTPPYNPSTDYTTDYTVVT |
| | | EYTTYCPEPTTFTTNGKTYTVTEPTTLTITDCPCTIEKPTTTST |
| | | TEYTVVTEYTTYCPEPTTFTTNGKTYTVTEPTTLTITDCPCTIE |
| | | KSEAPESSVPVTESKGTTTKETGVTTKQTTANPSLTVSTVVP |
| | | VSSSASSHSVVINSNGANVVVPGALGLAGVAMLFL |
| 42 | S. cerevisiae | MKFSAGAVLSWSSLLLASSVFAQQEAVAPEDSAVVKLATDS |
| | PDI1 | FNEYIQSHDLVLAEFFAPWCGHCKNMAPEYVKAAETLVEKN |
| | (amino acids | ITLAQIDCTENQDLCMEHNIPGFPSLKIFKNSDVNNSIDYEGP |
| | 1-28 signal | RTAEAIVQFMIKQSQPAVAVVADLPAYLANETFVTPVIVQSG |
| | sequence) | KIDADFNATFYSMANKHFNDYDFVSAENADDDFKLSIYLPS |
| | | AMDEPVVYNGKKADIADADVFEKWLQVEALPYFGEIDGSVF |
| | | AQYVESGLPLGYLFYNDEEELEEYKPLFTELAKKNRGLMNF |
| | | VSIDARKFGRHAGNLNMKEQFPLFAIHDMTEDLKYGLPQLS |
| | | EEAFDELSDKIVLESKAIESLVKDFLKGDASPIVKSQEIFENQD |
| | | SSVFQLVGKNHDEIVNDPKKDVLVLYYAPWCGHCKRLAPT |
| | | YQELADTYANATSDVLIAKLDHTENDVRGVVIEGYPTIVLYP |
| | | GGKKSESVVYQGSRSLDSLFDFIKENGHFDVDGKALYEEAQ |
| | | EKAAEEADADAELADEEDAIHDEL |

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1: An antibody display system comprising
(a) an isolated yeast host cell;
(b) a polynucleotide encoding a bait comprising an immunoglobulin heavy chain Fc domain fused to a cell surface anchor polypeptide, which said cell surface anchor polypeptide comprises more than 320 amino acids, operably linked to a regulatable promotor;
(c) one or more polynucleotides encoding an immunoglobulin light chain variable domain (VL); and
(d) one or more polynucleotides encoding an immunoglobulin heavy chain variable domain (VH).

2: The antibody display system of claim 1, wherein the antibody display system further comprises
(i) a non-tethered full-length bivalent antibody tetramer comprising two immunoglobulin heavy chains (HC), each HC comprising said VH, and two immunoglobulin light chains (LC), each LC comprising said VL; and/or
(ii) a monovalent antibody fragment comprising one HC and one LC complexed with the Fc moiety of the bait.

3: The antibody display system of claim 1, wherein said one or more polynucleotides encoding a VL is from a diverse population of VLs; and/or, wherein said one or more polynucleotides encoding a VH is from a diverse population of VHs.

4: The antibody display system of claim 1, wherein the VL is fused to an immunoglobulin light chain constant domain and the VH is fused to an immunoglobulin heavy chain constant domain having an Fc domain or immunoglobulin heavy chain CH1 domain and lacking an Fc domain.

5: The antibody display system of claim 4, wherein the immunoglobulin heavy chain constant domain is an IgG1, IgG2, IgG3, or IgG4 immunoglobulin constant domain.

6: The antibody display system of claim 1, wherein the Fc immunoglobulin domain is an IgG1, IgG2, IgG3 or IgG4 Fc immunoglobulin domain.

7: The antibody display system of claim 1, wherein the surface anchor polypeptide comprises between 400 to 700 amino acids.

8: The antibody display system of claim 1, wherein the regulatable promoter is a TetO7 promoter.

9: The antibody display system of claim 1, wherein the surface anchor polypeptide comprises a Saccharomyces cerevisiae SED1 protein.

10: The antibody display system of claim 1, wherein the surface anchor polypeptide comprises a Saccharomyces cerevisiae SED1 protein comprising about 401, 430, or 481 amino acids.

11. (canceled)

12: The antibody display system of claim 1, wherein the cell surface anchor polypeptide is a chimeric surface anchor polypeptide comprising a Saccharomyces cerevisiae SED1 protein and a heterologous protein.

13. (canceled)

14. The antibody display system of claim 12, wherein the heterologous protein amino acid sequence is a minisatellite-like repeat sequence from a yeast cell wall protein.

15: The antibody display system of claim 14, wherein the yeast cell wall protein is selected from FLO1, FLO2, and FLO11.

16-23. (canceled)

24: A method for the selection of a yeast diploid cell that secretes an antibody tetramer that selectively binds a molecule of interest, the method comprising:
(a) transforming a multiplicity of yeast haploid cells with (i) a first polynucleotide, said first polynucleotide encoding an Fc bait polypeptide comprising the Fc domain of an antibody heavy chain constant domain fused to a cell surface anchor polypeptide, which said cell surface anchor polypeptide comprises more than 320 amino acids, operably linked to a first regulatable promoter, and (ii) a plurality of second polynucleotides, each second polynucleotide independently encoding an antibody heavy chain variable domain (VH), operably linked to a second regulatable promoter, to provide a plurality of first yeast haploid cells;
(b) transforming a multiplicity of yeast haploid cells with a plurality of polynucleotides, each third polynucleotide independently encoding a light chain variable domain (VL), operably linked to the second regulatable promoter, to provide a plurality of second yeast haploid cells;
(c) generating a plurality of yeast diploid cells from said first and second yeast haploid cells;
(d) culturing said plurality of yeast diploid cells under a first condition wherein the Fc bait polypeptide and the antibody heavy and light chain variable domains are expressed and displayed on the surface of the diploid yeast cells in a complex comprising the Fc bait complexed to a monovalent antibody fragment comprising a heavy chain variable domain and a light chain variable domain;
(e) selecting those yeast diploid cells in the plurality of yeast diploid cells in which the monovalent antibody fragment selectively binds the molecule of interest to provide selected yeast diploid cells; and
(f) culturing at least one selected yeast diploid cell under a second condition wherein full-length bivalent antibody tetramers comprising two immunoglobulin heavy chains and two immunoglobulin light chains that specifically bind the molecule of interest are expressed and secreted from the selected yeast diploid cell and the Fc bait is not expressed.

25-27. (canceled)

28: The method of claim 24, wherein the cell surface anchor polypeptide comprises between 400 to 700 amino acids.

29: The method of claim 24, wherein the first regulatable promoter is a TetO7 promoter.

30: The method of claim 24, wherein the cell surface anchor polypeptide comprises a Saccharomyces cerevisiae SED1 protein.

31: The method of claim 24, wherein the cell surface anchor polypeptide comprises a Saccharomyces cerevisiae SED1 protein comprising about 401, 430, or 481 amino acids.

32-43. (canceled)

44: A diploid yeast host cell comprising
(a) a polynucleotide encoding a bait comprising an immunoglobulin Fc domain fused to a cell surface anchor polypeptide, which said cell surface anchor polypeptide comprises more than 320 amino acids, operably linked to a regulatable promotor;
(b) a polynucleotide encoding an immunoglobulin light chain variable domain (VL); and
(c) a polynucleotide encoding an immunoglobulin heavy chain variable domain (VH).

45-84. (canceled)
